SEQUENCE LISTING (78 sequences)

SEQ ID NO: 1 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 2 - protein, 40 aa, Homo sapiens
QEECVCENYKLAVNCFVNNNRQCQCTSVGAQNTVICSKLA

SEQ ID NO: 3 - protein, 113 aa, Mus musculus
QIQLVQSGPELKKPGETVKISCKTSGYTFTDYSMHWVNQAPGKGLKWMGWINTETGEPTYTDDFKGRFAFSLETSASTAYLQINNLKNEDTATYFCARTAVYWGQGTTLTVSS

SEQ ID NO: 4 - DNA, 339 bp, Mus musculus
cagatccagttggtgcagtctggacctgaactgaagaagcctggggagacagtcaagatctcctgcaagacttctggttataccttcacagactattcaatgcactgggtgaatcaggctcctggaaagggtttaaagtggatgggctggataaacactgagactggtgagccaacatatacagatgacttcaagggacggttcgccttctctttggagacctctgccagcactgcctatttacagatcaacaacctcaaaaatgaggacacggctacatatttctgtgctaggactgcggtctactggggccaaggcaccactctcacagtctcctcc

SEQ ID NO: 5 - protein, 108 aa, Mus musculus
DIQMTQSPSSLSASLGERVSLTCRASQEISGSLSWLQQKPDGTIKRLIYAASTLNSGVPKRFSGRRSGSDYSLTISSLESEDFVDYYCLQYSSYPWSFGGGTKLEIKR

SEQ ID NO: 6 - DNA, 324 bp, Mus musculus
gacatccagatgacccagtctccatcctccttatctgcctctctgggagaaagagtcagtctcacctgtcgggcaagccaggaaattagtggttccttaagctggcttcagcagaaaccagatggaactattaaacgcctgatctacgccgcttccactttaaattctggtgtcccaaaaaggttcagtggtagaaggtctgggtcagattattctctcaccatcagcagccttgagtctgaagattttgtagactattactgtctacaatattctagttatccgtggtcgttcggtggaggcaccaagctggaaatcaaacgg

SEQ ID NO: 7 - protein, 116 aa, Mus musculus
QIQLVQSGPELKKPGETVKISCKASGYTFTNYGMNWVKQAPGKGFKWMGWINTYTGEPTYADDFKGRFAFSLETSASTAYLQINNLQNEDMATYFCARWGYDFDYWGQGTTLTVSS

SEQ ID NO: 8 - DNA, 348 bp, Mus musculus
cagatccagttggtgcagtctggacctgagctgaagaagcctggagagacagtcaagatctcctgcaaggcttctgggtataccttcacaaactatggaatgaactgggtgaagcaggctccaggaaagggttttaagtggatgggctggataaacacctacactggagagccaacatatgctgatgacttcaagggacggtttgccttctctttggaaacctctgccagcactgcctatttgcagatcaacaacctccaaaatgaggacatggctacatatttctgtgcaagatgggggtacgactttgactactggggccaaggcaccactctcacagtctcctca

SEQ ID NO: 9 - protein, 107 aa, Mus musculus
QIVLTQSPAIMSSSPGEKVTMTCSASSSISYMNWYQQKPGTSPKRWIYDTSKLASGVPARFSGSGSGTSYSLTISSMEAEDAATYYCHQRSSYPYTFGGGTKLEIKR

SEQ ID NO: 10 - DNA, 321 bp, Mus musculus
caaattgtcctcacccagtctccagcaatcatgtcttcatctccaggggagaaggtcaccatgacctgcagtgccagctcaagtataagttacatgaactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacccgtacacgttcggaggggggaccaagctggaaataaaacgg

SEQ ID NO: 11 - protein, 116 aa, Mus musculus
EVKLLESGGGLVQPGGSLKLSCAASGFDFSRYWMSWVRQAPGKGLEWIGEINPDSSTMNYTPSLKDKFIISRDNAKNTLYLQMSKVRSEDTALYYCARGAYAMDYWGQGTSVTVSS

SEQ ID NO: 12 - DNA, 348 bp, Mus musculus
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgatgaactatacgccatctctaaaggataaattcatcatctccagagacaacgccaaaaatacgctgtacctgcaaatgagcaaagtgagatctgaggacacagccctttattactgtgcaaggggggcctatgctatggactactggggtcaaggaacctcagtcaccgtctcctca

SEQ ID NO: 13 - protein, 110 aa, Mus musculus
QAVVTQESALTTSPGETVTLTCRSSTGAVTTSNNANWVQEKPDHLFTGLIGATNNRAPGVPARFSGSLIGDKAALTITGAQTEDEAIYFCALWYINHWVFGGGTKLTVLG

SEQ ID NO: 14 - DNA, 330 bp, Mus musculus
caggctgttgtgactcaggaatctgcactcaccacatcacctggtgaaacagtcacactcacttgtcgctcaagtactggggctgttacaactagtaacaatgccaactgggtccaagaaaaaccagatcatttattcactggtctaataggtgcaaccaacaaccgagctccaggtgttcctgccagattctcaggctccctgattggagacaaggctgccctcaccatcacaggggcacagactgaggatgaggcaatatatttctgtgctctatggtacatcaaccactgggtgttcggtggaggaaccaaactgactgtcctaggc

SEQ ID NO: 15 - protein, 118 aa, Mus musculus
QIQLVQSGPELKKPGETVKISCKASGYTFTNYGMNWVKQAPGKGLKWMGWINTNTGEATYVEEFKGRVVFSLETSASTAYLQINNLKNEDTATYFCARIGYVWYFDVWGAGTTVTVSS

SEQ ID NO: 16 - DNA, 354 bp, Mus musculus
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagaggcaacatatgttgaagagttcaagggacgggttgtcttctctttggaaacctctgccagcactgcctatttgcagatcaacaacctcaaaaatgaggacacggctacatatttctgtgcaagaatcggttacgtctggtacttcgatgtctggggcgcagggaccacggtcaccgtctcctca

SEQ ID NO: 17 - protein, 113 aa, Mus musculus
DIVMSQSPSSLAVSAGEKVTMSCKSSQSLFSSRTRKNYLAWYQQKPGQSPKLLIYWASTRESGVPDRFTGSGSGTDFTLTINSVQAEDLAVYYCKHSNNLFTFGSGTKLEIKR

SEQ ID NO: 18 - DNA, 339 bp, Mus musculus
gacattgtgatgtcacagtctccatcctccctggctgtgtcagcaggagagaaggtcactatgagctgcaaatccagtcagagtctgttcagcagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcaacagtgtgcaggctgaagacctggcagtttattactgcaaacattctaataatctattcacgttcggctcggggacaaagttggaaataaaacgg

SEQ ID NO: 19 - protein, 122 aa, Mus musculus
EVQLVESGGDLVKPGGSLKLSCAASGFAFDSYGMSWVRQTPGKRLEWVATISSGGYYTYYQDSVKGRFTTSRDNAKNTLDLQMSSLKSEDSAIYYCARRGNYDVDYYAMDYWGQGTSVTVSS

SEQ ID NO: 20 - DNA, 366 bp, Mus musculus
gaggtgcagctggtggagtctgggggagacttagtgaagcctggagggtccctgaaactctcctgtgcagcctctggattcgctttcgatagttatggcatgtcttgggttcgccagactccaggcaagaggctggagtgggtcgcaaccattagtagtggtggttattacacctactatcaagacagtgtgaagggacgattcaccacctccagagacaatgccaagaacaccctggacctccaaatgagcagtctgaagtctgaggactcagccatttattactgtgcaagacgggggaattacgacgtagattactatgctatggactactggggtcaaggaacctcagtcaccgtctcctca

SEQ ID NO: 21 - protein, 114 aa, Mus musculus
DVVMTQTPLSLPVSLGDQASISCRSSQSLVHSNGNTYLHWYLQKPGQSPKLLIYKVSNRFSGVPDRFSGSGSGTDFTLRISRVEAEDLGVYFCSQSTHVPPWTFGGGTKLEIKR

SEQ ID NO: 22 - DNA, 342 bp, Mus musculus
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaggatcagcagagtggaggctgaggatctgggagtttatttctgctctcaaagtacacatgttcctccgtggacgttcggtggaggcaccaagttggaaatcaaacgg

SEQ ID NO: 23 - protein, 122 aa, Mus musculus
EVQLVESGGDLVKPGGSLKLSCAASGFAFDSYGMSWIRQTPGTRLEWVATISSGGYYTYYEDNVKGRFIISRDNAKNTLDLQMSGLKSEDAATYYCARRGNYDVDYYAMDYWGQGTSVIVSS

SEQ ID NO: 24 - DNA, 366 bp, Mus musculus
gaggtgcagctggtggagtctgggggagacttagtgaagcctggagggtccctgaaactctcctgtgcagcctctggattcgctttcgatagctatggcatgtcttggattcgccagactccaggcacgaggctggagtgggtcgcaaccattagtagtggtggttattacacctattatgaagacaatgtgaaggggcgattcattatctccagagacaatgccaagaacaccctggacctccaaatgagcggtctgaagtctgaggacgcagccacttattactgtgcaagacgggggaattacgacgtagattactatgctatggactactggggtcaaggaacctcagtcatcgtctcctca

SEQ ID NO: 25 - protein, 114 aa, Mus musculus
DVVLTQIPLSLPVSLGDQASISCRSSQSLEHSNGNTYLHWYLQKPGQSPKLLIYKVSNRFSGVPDRFSGSGSGTDFTLKISRVEAEDLGVYFCSQSTHVPPWTFGGGTKLEFKR

SEQ ID NO: 26 - DNA, 342 bp, Mus musculus
gatgttgtgctgacccaaattccactctccctgcctgtcagtcttggagatcaagcctccatctcttgcagatctagtcagagccttgaacacagtaatggaaacacctatttacattggtacctgcagaagccaggccagtctccaaagctcctaatttacaaagtttccaaccgattttctggggtcccagacaggttcagtggcagtggatcagggacagacttcacactcaagatcagcagagtggaggctgaggatctgggagtttatttctgctctcaaagtacacatgttcctccgtggacgttcggtggaggcaccaagttggaattcaaacgg

SEQ ID NO: 27 - protein, 113 aa, Artificial Sequence
  Synthetic Construct
EIQLVQSGGGLVKPGGSVRISCATSGYTFTDYSMHWVRQAPGKGLEWMGWINTETGEPTYTDDFKGRFTFSLDDSKNTAYLQINSLRAEDTAVYFCARTAVYWGQGTTLTVSS

SEQ ID NO: 28 - DNA, 339 bp, Artificial Sequence
  Synthetic Construct
gagatccagttggtgcagtctggaggaggcctggtaaagcctggggggtccgtcagaatctcctgcgcaacttctggttataccttcacagactattcaatgcactgggtgcgccaggctcctggaaagggtttagagtggatgggctggataaacactgagactggtgagccaacatatacagatgacttcaagggacggttcaccttctctttggacgactctaagaacactgcctatttacagatcaacagcctcagagccgaggacacggctgtgtatttctgtgctaggactgcggtctactggggccaaggcaccactctcacagtctcctcc

SEQ ID NO: 29 - protein, 107 aa, Artificial Sequence
  Synthetic Construct
DIQMTQSPSSLSASVGDRVSLTCRASQEISGSLSWLQQKPDGAIKRLIYAASTLNSGVPKRFSGRRSGTDYTLTISSLQSEDFVDYYCLQYSSYPWSFGGGTKLEIK

SEQ ID NO: 30 - DNA, 321 bp, Artificial Sequence
  Synthetic Construct
gacatccagatgacccagtctccatcctccttatctgcctctgtaggagacagagtcagtctcacctgtcgggcaagccaggaaattagtggttccttaagctggcttcagcagaaaccagatggagccattaaacgcctgatctacgccgcttccactttaaattctggtgtcccaaaaaggttcagtggtagaaggtctgggacagattatactctcaccatcagcagccttcagtctgaagattttgtagactattactgtctacaatattctagttatccgtggtcgttcggtggaggcaccaagctggaaatcaaa

SEQ ID NO: 31 - protein, 113 aa, Artificial Sequence
  Synthetic Construct
QVQLVQSGSELKKPGASVKISCETSGYTFTDYSMHWVRQAPGQGLEWMGWINTETGEPTYTDDFKGRFVFSLDTSVSTAYLQISSLKAEDTAVYYCARTAVYWGQGTLVTVSS

SEQ ID NO: 32 - DNA, 339 bp, Artificial Sequence
  Synthetic Construct
caggtgcagttggtgcagtctggatctgaactgaagaagcctggggcctcagtcaagatctcctgcgagacttctggttataccttcacagactattcaatgcactgggtgcgacaggctcctggacaaggtttagagtggatgggctggataaacactgagactggtgagccaacatatacagatgacttcaagggacggttcgtcttctctttggacacctctgtcagcactgcctatttacagatcagcagcctcaaagctgaggacacggctgtgtattactgtgctaggactgcggtctactggggccaaggcaccctggtcacagtctcctcc

SEQ ID NO: 33 - protein, 107 aa, Artificial Sequence
  Synthetic Construct
DIQMTQSPSSLSASVGDRVTITCRASQEISGSLSWYQQKPGKAPKRLIYAASTLNSGVPSRFSGSRSGTEFTLTISSLQPEDFATYYCLQYSSYPWSFGGGTKVEIK

SEQ ID NO: 34 - DNA, 321 bp, Artificial Sequence
  Synthetic Construct
gacatccagatgacccagtctccatcctccttatctgcctctgtaggagacagagtcaccatcacctgtcgggcaagccaggaaattagtggttccttaagctggtatcagcagaaaccagggaaagcccctaaacgcctgatctacgccgcttccactttaaattctggtgtcccatcaaggttcagtggtagtaggtctgggacagaattcactctcaccatcagcagccttcagcctgaagattttgcaacttattactgtctacaatattctagttatccgtggtcgttcggtggaggcaccaaggtggaaatcaaa

SEQ ID NO: 35 - protein, 118 aa, Artificial Sequence
  Synthetic Construct
EVQLVESGGGLVQPGGSLRLSCAASGYTFTNYGMNWIRQAPGKGLEWVGWINTNTGEATYVEEFKGRFTISLDTSASTVYLQMNSLRAEDTAVYYCARIGYVWYFDVWGQGTLVTVSS

SEQ ID NO: 36 - DNA, 354 bp, Artificial Sequence
  Synthetic Construct
gaggttcagctggtggagtctggcggtggcctggtgcagccagggggctcactccgtttgtcctgtgcagcttctggctacacgtttaccaactatggaatgaactggatacgtcaggccccaggtaagggcctggaatgggttggctggataaacaccaacactggagaggcaacatatgttgaagagttcaagggacgtttcacgatatccctggatacgtccgccagcacagtctacctgcaaatgaacagcctgcgtgctgaggacactgccgtctattattgtgctagaatcggttacgtctggtacttcgatgtctggggtcaaggaaccctggtcaccgtctcctcg

SEQ ID NO: 37 - protein, 112 aa, Artificial Sequence
  Synthetic Construct
DIQMTQSPSSLSASVGDRVTITCKSSQSLFSSRTRKNYLAWYQQKPGKAPKLLIYWASTRESGVPSRFSGSGSGTDFTFTISSLQPEDIATYYCKHSNNLFTFGQGTKVEIK

SEQ ID NO: 38 - DNA, 336 bp, Artificial Sequence
  Synthetic Construct
gacatccagatgacccagagcccaagcagcctgagcgccagcgtgggtgacagagtgaccatcacctgtaaatccagtcagagtctgttcagcagtagaacccgaaagaactacttggcttggtaccagcagaagccaggtaaggctccaaagctgctgatctactgggcatccactagggaatctggtgtgccaagcagattcagcggtagcggtagcggtaccgacttcaccttcaccatcagcagcctccagccagaggacatcgccacctactactgcaaacattctaataatctattcacgttcggccaagggaccaaggtggaaatcaaa

SEQ ID NO: 39 - protein, 489 aa, Artificial Sequence
  Synthetic Construct
DIQMTQSPSSLSASVGDRVSLTCRASQEISGSLSWLQQKPDGAIKRLIYAASTLNSGVPKRFSGRRSGTDYTLTISSLQSEDFVDYYCLQYSSYPWSFGGGTKLEIKGGGGSGGGGSGGGGSEIQLVQSGGGLVKPGGSVRISCATSGYTFTDYSMHWVRQAPGKGLEWMGWINTETGEPTYTDDFKGRFTFSLDDSKNTAYLQINSLRAEDTAVYFCARTAVYWGQGTTLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 40 - DNA, 1467 bp, Artificial Sequence
  Synthetic Construct
gacatccagatgacccagtctccatcctccttatctgcctctgtaggagacagagtcagtctcacctgtcgggcaagccaggaaattagtggttccttaagctggcttcagcagaaaccagatggagccattaaacgcctgatctacgccgcttccactttaaattctggtgtcccaaaaaggttcagtggtagaaggtctgggacagattatactctcaccatcagcagccttcagtctgaagattttgtagactattactgtctacaatattctagttatccgtggtcgttcggtggaggcaccaagctggaaatcaaaggtggtggtggttctggcggcggcggctccggtggtggtggttctgagatccagttggtgcagtctggaggaggcctggtaaagcctggggggtccgtcagaatctcctgcgcaacttctggttataccttcacagactattcaatgcactgggtgcgccaggctcctggaaagggtttagagtggatgggctggataaacactgagactggtgagccaacatatacagatgacttcaagggacggttcaccttctctttggacgactctaagaacactgcctatttacagatcaacagcctcagagccgaggacacggctgtgtatttctgtgctaggactgcggtctactggggccaaggcaccactctcacagtctcctccggtggaggcggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaacatcatcaccatcatcat

SEQ ID NO: 41 - protein, 489 aa, Artificial Sequence
  Synthetic Construct
DIQMTQSPSSLSASVGDRVTITCRASQEISGSLSWYQQKPGKAPKRLIYAASTLNSGVPSRFSGSRSGTEFTLTISSLQPEDFATYYCLQYSSYPWSFGGGTKVEIKGGGGSGGGGSGGGGSQVQLVQSGSELKKPGASVKISCETSGYTFTDYSMHWVRQAPGQGLEWMGWINTETGEPTYTDDFKGRFVFSLDTSVSTAYLQISSLKAEDTAVYYCARTAVYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 42 - DNA, 1467 bp, Artificial Sequence
  Synthetic Construct
gacatccagatgacccagtctccatcctccttatctgcctctgtaggagacagagtcaccatcacctgtcgggcaagccaggaaattagtggttccttaagctggtatcagcagaaaccagggaaagcccctaaacgcctgatctacgccgcttccactttaaattctggtgtcccatcaaggttcagtggtagtaggtctgggacagaattcactctcaccatcagcagccttcagcctgaagattttgcaacttattactgtctacaatattctagttatccgtggtcgttcggtggaggcaccaaggtggaaatcaaaggtggtggtggttctggcggcggcggctccggtggtggtggttctcaggtgcagttggtgcagtctggatctgaactgaagaagcctggggcctcagtcaagatctcctgcgagacttctggttataccttcacagactattcaatgcactgggtgcgacaggctcctggacaaggtttagagtggatgggctggataaacactgagactggtgagccaacatatacagatgacttcaagggacggttcgtcttctctttggacacctctgtcagcactgcctatttacagatcagcagcctcaaagctgaggacacggctgtgtattactgtgctaggactgcggtctactggggccaaggcaccctggtcacagtctcctccggtggaggcggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaacatcatcaccatcatcat

SEQ ID NO: 43 - protein, 499 aa, Artificial Sequence
  Synthetic Construct
DIQMTQSPSSLSASVGDRVTITCKSSQSLFSSRTRKNYLAWYQQKPGKAPKLLIYWASTRESGVPSRFSGSGSGTDFTFTISSLQPEDIATYYCKHSNNLFTFGQGTKVEIKGGGGSGGGGSGGGGSEVQLVESGGGLVQPGGSLRLSCAASGYTFTNYGMNWIRQAPGKGLEWVGWINTNTGEATYVEEFKGRFTISLDTSASTVYLQMNSLRAEDTAVYYCARIGYVWYFDVWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 44 - DNA, 1499 bp, Artificial Sequence
  Synthetic Construct
gacatccagatgacccagagcccaagcagcctgagcgccagcgtgggtgacagagtgaccatcacctgtaaatccagtcagagtctgttcagcagtagaacccgaaagaactacttggcttggtaccagcagaagccaggtaaggctccaaagctgctgatctactgggcatccactagggaatctggtgtgccaagcagattcagcggtagcggtagcggtaccgacttcaccttcaccatcagcagcctccagccagaggacatcgccacctactactgcaaacattctaataatctattcacgttcggccaagggaccaaggtggaaatcaaaggtggtggtggttctggcggcggcggctccggtggtggtggttctgaggttcagctggtggagtctggcggtggcctggtgcagccagggggctcactccgtttgtcctgtgcagcttctggctacacgtttaccaactatggaatgaactggatacgtcaggccccaggtaagggcctggaatgggttggctggataaacaccaacactggagaggcaacatatgttgaagagttcaagggacgtttcacgatatccctggatacgtccgccagcacagtctacctgcaaatgaacagcctgcgtgctgaggacactgccgtctattattgtgctagaatcggttacgtctggtacttcgatgtctggggtcaaggaaccctggtcaccgtctcctcgggtggaggcggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggctctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaacatcatcaccatcatcattag

SEQ ID NO: 45 - protein, 585 aa, Homo sapiens [sequence omitted]

SEQ ID NO: 46 - DNA, 1755 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 47 - protein, 205 aa, Homo sapiens [sequence omitted]

SEQ ID NO: 48 - DNA, 615 bp, Homo sapiens
gtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 49 - protein, 15 aa, Artificial Sequence
  Synthetic Construct
GGGGSGGGGSGGGGS

SEQ ID NO: 50 - DNA, 45 bp, Artificial Sequence
  Synthetic Construct
ggtggtggtggttctggcggcggcggctccggtggtggtggttct

SEQ ID NO: 51 - protein, 5 aa, Artificial Sequence
  Synthetic Construct
GGGGS

SEQ ID NO: 52 - DNA, 15 bp, Artificial Sequence
  Synthetic Construct
ggtggaggcggatcc

SEQ ID NO: 53 - protein, 18 aa, Artificial Sequence
  Synthetic Construct
VEGGSGGSGGSGGSGGVD

SEQ ID NO: 54 - DNA, 54 bp, Artificial Sequence
  Synthetic Construct
gtcgaaggtggaagtggaggttctggtggaagtggaggttcaggtggagtcgac

SEQ ID NO: 55 - protein, 119 aa, Mus musculus
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 56 - DNA, 357 bp, Mus musculus
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctca

SEQ ID NO: 57 - protein, 106 aa, Mus musculus
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 58 - DNA, 318 bp, Mus musculus
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 59 - protein, 19 aa, Artificial Sequence
  Synthetic Construct
MKLPVRLLVLMFWIPVSSS

SEQ ID NO: 60 - DNA, 57 bp, Artificial Sequence
  Synthetic Construct
atgaaattgcctgttaggctgttggtgctgatgttctggattcctgtttccagcagt

SEQ ID NO: 61 - protein, 20 aa, Artificial Sequence
  Synthetic Construct
METDTLLLWVLLLWVPGSTG

SEQ ID NO: 62 - DNA, 60 bp, Artificial Sequence
  Synthetic Construct
atggagacagacacactcctgctatgggtgctgctgctctgggttccaggttccaccggt

SEQ ID NO: 63 - protein, 4 aa, Artificial Sequence
  Synthetic Construct
GGGS

SEQ ID NO: 64 - protein, 10 aa, Artificial Sequence
  Synthetic Construct
GGGGSGGGGS

SEQ ID NO: 65 - protein, 20 aa, Artificial Sequence
  Synthetic Construct
GGGGSGGGGSGGGGSGGGGS

SEQ ID NO: 66 - protein, 25 aa, Artificial Sequence
  Synthetic Construct
GGGGSGGGGSGGGGSGGGGSGGGGS

SEQ ID NO: 67 - protein, 20 aa, Artificial Sequence
  Synthetic Construct
GGGGSGGRASGGGGSGGGGS

SEQ ID NO: 68 - protein, 6 aa, Artificial Sequence
  Synthetic Construct
STDGNT

SEQ ID NO: 69 - protein, 5 aa, Artificial Sequence
  Synthetic Construct
GGSGG

SEQ ID NO: 70 - protein, 6 aa, Artificial Sequence
  Synthetic Construct
SAKTTP

SEQ ID NO: 71 - protein, 10 aa, Artificial Sequence
  Synthetic Construct
SAKTTPKLGG

SEQ ID NO: 72 - protein, 6 aa, Artificial Sequence
  Synthetic Construct
RADAAP

SEQ ID NO: 73 - protein, 9 aa, Artificial Sequence
  Synthetic Construct
RADAAPTVS

SEQ ID NO: 74 - protein, 12 aa, Artificial Sequence
  Synthetic Construct
RADAAAAGGPGS

SEQ ID NO: 75 - protein, 27 aa, Artificial Sequence
  Synthetic Construct
RADAAAAGGGGSGGGGSGGGGSGGGGS

SEQ ID NO: 76 - protein, 20 aa, Artificial Sequence
  Synthetic Construct
GGKGSGGKGTGGKGSGGKGS

SEQ ID NO: 77 - protein, 8 aa, Artificial Sequence
  Synthetic Construct
AAAGGSGG

SEQ ID NO: 78 - protein, 15 aa, Artificial Sequence
  Synthetic Construct
GGGGSGGRASGGGGS

(19) United States
(12) Patent Application Publication
(10) Pub. No.: US 2011/0165161 A1
(43) Pub. Date: Jul. 7, 2011

(54) ANTI-EPCAM ANTIBODIES THAT INDUCE APOPTOSIS OF CANCER CELLS AND METHODS USING SAME

(76) Inventors: Shih-Yao LIN, Taipei (TW); Leewen Lin, Taipei (TW); Feng-Lin Chiang, Taipei (TW); Shu-Hua Lee, Taipei (TW); Yu-Ying Tsai, Taipei (TW)

(21) Appl. No.: 12/977,976

(22) Filed: Dec. 23, 2010

Related U.S. Application Data
(60) Provisional application No. 61/289,729, filed on Dec. 23, 2009.
(60) Provisional application No. 61/294,008, filed on Jan. 11, 2010.

(51) Int. Cl. A61K 39/395; C07K 16/32; C07H 21/00; C12N 15/63; C12N 5/10; C12N 1/00; C12P 21/02; G01N 33/574; A61P 35/00

(52) U.S. Cl. 424/135.1; 530/387.7; 530/387.3; 424/133.1; 424/138.1; 536/23.53; 435/320.1; 435/325; 435/243; 435/69.6; 435/7.23

(57) ABSTRACT

The present invention provides antibodies (such as chimeric and humanized antibodies) specifically bind to epithelial cell adhesion/activating molecule EpCAM expressed on cancer cells and induce cancer cell apoptosis. In addition, the present invention also provides use of the antibodies described herein for diagnostic and therapeutic purposes.

### CROSS-REFERENCE TO RELATED APPLICATIONS

This application claims the priority benefit of U.S. provisional applications U.S. Ser. No. 61/289,729, filed Dec. 23, 2009, and U.S. Ser. No. 61/294,008, filed Jan. 11, 2010, all of which are incorporated herein by reference in their entirety.

### FIELD OF THE INVENTION

The present invention relates to antibodies that recognize human epithelia cell adhesion/activating molecule (EpCAM) expressed on cancer cells. These antibodies have the property of inducing cell death (e.g., apoptosis) in these cancer cells in the absence of cytotoxin conjugation and immune effector function. These antibodies are useful as diagnostic and therapeutic agents.

### BACKGROUND OF THE INVENTION

Epithelial cell adhesion/activating molecule (EpCAM/CD326, also known as 17-1A antigen, HEA125, MK-1, EGP-2, EGP34, GA733-2, KSA, TROP-1, KS1/4 and ESA) is one of the first and most importance immunotherapeutic targets in cancer therapy, due to its high-level and frequent expression on most carcinomas of different origin (Herlyn et al. (1979) Proc Natl Acad Sci USA 76:1438-1442; Went et al. (2004) Hum Pathol 35:122-128). This antigen is a relatively small type I transmembrane glycoprotein of 314 amino acids (aa) that is highly conserved during evolution and is reported to mediate calcium-independent homotypic cell-cell adhesions (Litvinov et al. (1994) J Cell Biology 125:437-446). The molecule consists of a short intracellular domain of 26 aa in which two binding sites for α-actinin are present for linkage to the actin cytoskeleton (Balzar et al. (1998) Mol Cell Biol. 18(8): 4833-4843), a 23-aa transmembrane domain, a 242-aa extracellular domain, and a 23-aa signal peptide which is cleaved from the mature protein. The extracellular domain of EpCAM antigen has three N-linked glycosylation sites. Differential glycosylation status between normal and malignant tissues has been reported in certain types of cancer (Pauli et al. (2003) Cancer Lett 193:25-32). The current model of its tertiary extracellular structure contains 3 domains. The first two were believed to resemble epidermal growth factor (EGF)-like repeats in which twelve cysteine residues exist among them (Balzar et al. (2001) Mol Cell Biol 21:2570-2580). However, some study suggested that the second EGF-like repeat of EpCAM is in fact a thyroglobulin (TY) domain (Linnenbach et al. (1989) Proc Natl Acad Sci USA 86:27-31; Chong and Speicher (2001) J Biol Chem 276:5804-5813). The third domain is a unique cysteine-poor region (CPR) unrelated to any known molecules (Baeuerle P A and Gires O (2007) Br J Cancer 96:417-423).

EpCAM expression in human is epithelia-specific. The majority of epithelial cells express EpCAM, except squamous epithelium and some specific epithelium cell types, such as epidermal keratinocytes, hepatocytes, gastric parietal cells, and myoepithelial cells (Balzar et al. (1999) J Mol Med 77: 699-712; Momburg et al. (1987) Cancer Res 47:2883-2891). It is expressed at the basolateral membrane of normal epithelial cells. In tumor of epithelium origin, generally a higher expression level is observed (Balzar et al. (1999) J Mol Med 77: 699-712; Winter et al. (2003) Am J Pathol 163:2139-2148; Went et al. (2004) Hum Pathol 35:122-128; Went et al. (2006) Br J Cancer 94:128-135). Since epithelial cells are known to be the most important cell type in the development of human malignancies, with more than 90% of all malignant tumors are of epithelial origin (Birchmeiera et al. (1996) Acta Anatomica; 156 (3); 217-226), EpCAM is now considered to be one of the most frequently and intensely expressed tumor-associated antigens and has many times been independently discovered as immunogenic tumor-associated antigen for monoclonal antibodies (Gottlinger et al. (1986) Int J Cancer 38:47-53; Edwards et al. (1986) Cancer Res 46:1306-1317; Spurr et al. (1986) Int J Cancer 38:631-636; Momburg et al. (1987) Cancer Res 47:2883-2891; Schön et al. (1994) J Investig Dermatol 102: 987-991; Bumol et al. (1988) Hybridoma 7:407-415; Quak et al. (1990) Hybridoma 9:377-387). It has been found that the protein is expressed on a great variety of human adenocarcinoma and squamous cell carcinoma (Went et al. (2004) Hum Pathol 35:122-128). Recent study using immunohistochemistry (IHC) staining together with microarrays technology has analyzed fairly large sample numbers from patients with breast, ovarian, renal, esophageal, colon, gastric, prostate and lung cancer (Spizzo et al. (2004) Breast Cancer Res Treat 86:207-213; Spizzo et al. (2006) Gynecol Oncol 103:483-488; Stoecklein et al. (2006) BMC Cancer 6:165; Kimura et al. (2007) Int J Oncol. 30:171-179; Went et al. (2005) Am J Surg Pathol 29:83-88; Went et al. (2006) Br J Cancer 94:128-135). The data underscore the potential utility of EpCAM as immunotherapeutic target for treatment of human cancers.

The presence of EpCAM on normal epithelia, albeit with lower density compared to tumor cells (Kim et al. (2004) Clin Cancer Res 10:5464-5471; Osta et al. (2004) Cancer Res 64:5818-5824) has been a persistent concern for target therapy with monoclonal anti-EpCAM antibodies. The assuring data comes from the studies with transgenic mice expressing human EpCAM under EpCAM-specific regulatory sequences. In the study it was shown that, although EpCAM is specifically expressed on normal epithelia, it is not accessible by i.v. administered antibody due to its compact structures and thus a restricted accessibility (McLaughlin et al. (2001) Cancer Res 61:4105-4111). Based on these data, EpCAM is considered a valid target for anti-tumor therapy with monoclonal antibodies.

Indeed, the first monoclonal antibody ever applied for human cancer therapy was in fact a murine IgG2a antibody called mAb 17-1A (later named edrecolomab and Panorexs) which recognized EpCAM (Sears et al. (1982) Lancet. 1(8275):762-765; Sears et al. (1984) J Biol Response Mod 3(2):138-150). Since then, edrecolomab and other EpCAM-specific murine, chimeric and humanized monoclonal antibodies were also tested pre-clinically and clinically either in the form of native (naked) antibody, hybrid bispecific (trifunctional) antibody or as conjugates with toxins, radioisotopes, or the cytokines (IL-2 or GM-CSF) for cancer treatment (Velders et al. (1994) Cancer Res 54(7):1753-1759; Raum et al. (2001) Cancer Immunol Immunother 50(3):141-150; Elias et al. (1994) Am J Respir Crit Care Med 150:1114-1122; Di Paolo et al. (2003) Clin Cancer Res 9:2837-2848; Andratschke et al. (2007) Anticancer Res 27(1A):431-436; Xiang et al. (1997) Cancer Res 57(21):4948-4955; Schanzer et al. (2006) J Immunother 29(5):477-488; Wimberger et al. (2003) Int J Cancer 105(2):241-248; Amann et al. (2008) Cancer Res 68(1):143-151). To date numerous different immunotherapeutic approaches targeting EpCAM are still currently in clinical trials (Baeuerle P A and Gires O (2007) Br J Cancer 96:417-423). Data from clinical trials have suggested that naked anti-EpCAM antibodies such as edrecolomab (17-1A; Panorexs) and adecatumumab (MT201) have only limited anti-tumor effect (Punt et al. (2002) Lancet 360: 671-677; Micromet, Inc (2006) Final Data from Two Phase II Trials Indicate Activity of Adecatumumab (MT201) in Breast and Prostate Cancer. Press Release), likely through the activation of complement system (CDC) and antibody-dependent cytotoxicity (ADCC) effect (Schwartzberg (2001) Crit Rev Oncol Hematol 40(1):17-24; Naundorf et al. (2002) Int J Cancer 100(1):101-110; Prang et al. (2005) Br J Cancer 92(2):342-349; Oberneder et al. (2006) Eur J Cancer 42(15):2530-2538). The antibodies conjugated with very potent effector mechanisms such as IL-2, PE toxin, or anti-CD3 seems to have better anti-tumor effect. However, some adverse effects also limited the systemic use of such anti-EpCAM antibodies (Baeuerle P A and Gires O (2007) Br J Cancer 96:417-423).

The fact of “insufficient” or “limited” antitumor efficacy demonstrated in current anti-EpCAM trials indicates that there is still a need for the development of anti-EpCAM antibodies with improved anti-tumor function.

All references, publications, and patent applications disclosed herein are hereby incorporated by reference in their entirety.

### BRIEF SUMMARY OF THE INVENTION

The present invention is based upon the discovery of anti-EpCAM monoclonal antibodies that can induce tumor cell apoptosis in various EpCAM-positive cancer cell lines besides its CDC and ADCC effector function. The beneficial efficacy of the antibody with additional apoptosis-inducing activity was demonstrated in mouse xenograft models with EpCAM-positive cancer cell lines.

The invention provides an isolated monoclonal antibody, which antibody specifically binds to an epitope within amino acids 24-63 of human EpCAM (SEQ ID NO:1), wherein the naked antibody induces apoptosis of human cancer cells after binding to the epitope on the cell surface of the cancer cells in vitro. In some embodiments, the apoptosis-inducing activity of the naked antibody to human lung cancer cell line NCI-H358 is at least about 90% of the activity of an antibody selected from the group consisting of 12H8, 1F10, 1G10, 2D11, 6D11, and 4D2, wherein the apoptosis-inducing activity is measured by incubating the human lung cancer cell with an antibody at concentration of about 10 ug/ml and an incubation time of about 16-20 hours. The amino acid sequences of the heavy and light chain variable regions for 12H8, 1F10, 1G10, 2D11, 6D11, and 4D2 are shown in FIGS. 3-8. NCI-H358 is from bronchioalveolar carcinoma, and the cell line was deposited on Oct. 7, 2009 at the American Type Culture Collection with a Patent Deposit Designation of PTA-10386. The NCI-H358 is also available at ATCC having Accession No. CRL-5807. NCI-H358 was also deposited at Food Industry Research and Development Institute (Hsin-chu, Taiwan) with a Patent Deposit Designation of BCRC960419.

In some embodiments, the binding of the antibody to the epitope within amino acids 24-63 of human EpCAM depends on the presence of amino acid residues Q24, E25 and N42 of human EpCAM. In some embodiments, the binding of the antibody to the epitope within amino acids 24-63 of human EpCAM depends on the presence of amino acid residues Q24, E25, E26, N37, N41, Q47, and T49 of human EpCAM. In some embodiments, the binding of the antibody to the epitope within amino acids 24-63 of human EpCAM depends on the presence of amino acid residues E25, V40 and R44 of human EpCAM. In some embodiments, the binding of the antibody to the epitope within amino acids 24-63 of human EpCAM depends on the presence of amino acid residues N41, N43, and R44 of human EpCAM. In some embodiments, the binding of the antibody to the epitope within amino acids 24-63 of human EpCAM depends on the presence of amino acid residues Q24, E25, A35, F39, V40, N41, R44, Q45, and Q47 of human EpCAM.

In some embodiments, the invention provides an isolated monoclonal antibody that specifically binds to human EpCAM, comprising the three heavy chain complementary determining regions from SEQ ID NO:3, and/or the three light chain complementary determining regions from SEQ ID NO:5. In some embodiments, the invention provides an isolated monoclonal antibody that specifically binds to human EpCAM, comprising the three heavy chain complementary determining regions from SEQ ID NO:7, and/or the three light chain complementary determining regions from SEQ ID NO:9. In some embodiments, the invention provides an isolated monoclonal antibody that specifically binds to human EpCAM, comprising the three heavy chain complementary determining regions from SEQ ID NO:11, and/or the three light chain complementary determining regions from SEQ ID NO:13. In some embodiments, the invention provides an isolated monoclonal antibody that specifically binds to human EpCAM, comprising the three heavy chain complementary determining regions from SEQ ID NO:15, and/or the three light chain complementary determining regions from SEQ ID NO:17. In some embodiments, the invention provides an isolated monoclonal antibody that specifically binds to human EpCAM, comprising the three heavy chain complementary determining regions from SEQ ID NO:19, and/or the three light chain complementary determining regions from SEQ ID NO:21. In some embodiments, the invention provides an isolated monoclonal antibody that specifically binds to human EpCAM, comprising the three heavy chain complementary determining regions from SEQ ID NO:23, and/or the three light chain complementary determining regions from SEQ ID NO:25.

In some embodiments, the invention provides an isolated monoclonal antibody comprising a heavy chain variable region comprising the amino acid sequence of SEQ ID NO:3, and/or a light chain variable region comprising the amino acid sequence of SEQ ID NO:5. In some embodiments, the invention provides an isolated monoclonal antibody comprising a heavy chain variable region comprising the amino acid sequence of SEQ ID NO:7, and/or a light chain variable region comprising the amino acid sequence of SEQ ID NO:9. In some embodiments, the invention provides an isolated monoclonal antibody comprising a heavy chain variable region comprising the amino acid sequence of SEQ ID NO:11, and/or a light chain variable region comprising the amino acid sequence of SEQ ID NO:13. In some embodiments, the invention provides an isolated monoclonal antibody comprising a heavy chain variable region comprising the amino acid sequence of SEQ ID NO:15, and/or a light chain variable region comprising the amino acid sequence of SEQ ID NO:17. In some embodiments, the invention provides an isolated monoclonal antibody comprising a heavy chain variable region comprising the amino acid sequence of SEQ ID NO:19, and/or a light chain variable region comprising the amino acid sequence of SEQ ID NO:21. In some embodiments, the invention provides an isolated monoclonal antibody comprising a heavy chain variable region comprising the amino acid sequence of SEQ ID NO:23, and/or a light chain variable region comprising the amino acid sequence of SEQ ID NO:25.

In some embodiments, the antibody induces apoptosis of human cancer cells selected from the group consisting of breast cancer cells, colorectal cancer cells, gastric cancer cells, lung cancer cells, prostate cancer cells, pancreatic cancer cells, pharynx cancer cells, and ovarian cancer cells. In some embodiments, the antibody is a chimeric antibody, for example, comprising a heavy chain constant region and a light chain constant region from a human antibody. In some embodiments, the antibody is a humanized antibody. The invention also provides antigen-binding fragments of the anti-EpCAM antibodies described herein. In some embodiments, the antibody is a bispecific antibody comprising a first binding domain that specifically recognize human EpCAM, and a second binding domain that specifically recognize a different antigen. In some embodiments, the first binding domain of the bispecific antibodies comprises the three heavy chain complementary determining regions and/or the three light chain complementary determining regions of an anti-EpCAM antibody described herein. In some embodiments, the first binding domain of the bispecific antibody comprises the heavy chain variable region and/or the light chain variable region of an anti-EpCAM antibody described herein. In some embodiments, the second binding domain of the bispecific antibody specifically recognizes a CD3 (e.g., human CD3).

The invention also provides single-chain bispecific antibodies comprising (a) a first antigen binding domain that specifically binds to an epitope within amino acids 24-63 of human EpCAM, wherein the first antigen binding domain comprises a heavy chain variable region (V_{H}EpCAM) and a light chain variable region (V_{L}EpCAM); and (b) a second antigen binding domain that specifically binds to human CD3 antigen, wherein the second antigen binding domain comprises a heavy chain variable region (V_{H}CD3) and a light chain variable region (V_{L}CD3); wherein the variable regions are arranged from N-terminus to C-terminus in the order V_{L}EpCAM-V_{H}EpCAM-V_{H}CD3-V_{L}CD3.

In some embodiments, the V_{H}EpCAM and V_{L}EpCAM in the first antigen binding domain are derived from an antibody (e.g., an isolated monoclonal antibody), wherein the naked antibody induces apoptosis of human cancer cells after binding to an epitope (e.g., an epitope within amino acids 24-63 of human EpCAM) on the cell surface of the cancer cells in vitro. In some embodiments, the apoptosis-inducing activity of the naked antibody to human lung cancer cell line NCI-H358 is at least about 90% of the activity of an antibody selected from the group consisting of 12H8, 1F10, 1G10, 2D11, 6D11, and 4D2, wherein the apoptosis-inducing activity is measured by incubating the human lung cancer cell with an antibody at concentration of about 10 ug/ml and an incubation time of about 16-20 hours.

In some embodiments, the single-chain bispecific antibody further comprises a peptide linker between V_{L}EpCAM and V_{H}EpCAM, between V_{H}EpCAM and V_{H}CD3, and/or between V_{H}CD3 and V_{L}CD3. In some embodiments, the peptide linker between V_{L}EpCAM and V_{H}EpCAM comprises the amino acid sequence of SEQ ID NO:49. In some embodiments, the peptide linker between V_{H}CD3 and V_{L}CD3 comprises the amino acid sequence of SEQ ID NO:53. In some embodiments, the peptide linker between V_{H}EpCAM and V_{H}CD3 comprises the amino acid sequence of SEQ ID NO:51.

In some embodiments, the first antigen binding domain comprises the V_{H}EpCAM and the V_{L}EpCAM selected from the group consisting of: (a) the V_{H}EpCAM comprising the three CDRs from SEQ ID NO:3, and the V_{L}EpCAM comprising the three CDRs from SEQ ID NO:5; (b) the V_{H}EpCAM comprising the three CDRs from SEQ ID NO:7, and the V_{L}EpCAM comprising the three CDRs from SEQ ID NO:9; (c) the V_{H}EpCAM comprising the three CDRs from SEQ ID NO:11, and the V_{L}EpCAM comprising the three CDRs from SEQ ID NO:13; (d) the V_{H}EpCAM comprising the three CDRs from SEQ ID NO:15, and the V_{L}EpCAM comprising the three CDRs from SEQ ID NO:17; (e) the V_{H}EpCAM comprising the three CDRs from SEQ ID NO:19, and the V_{L}EpCAM comprising the three CDRs from SEQ ID NO:21; and (f) the V_{H}EpCAM comprising the three CDRs from SEQ ID NO:23, and the V_{L}EpCAM comprising the three CDRs from SEQ ID NO:25. In some embodiments, the V_{H}EpCAM and the V_{L}EpCAM are humanized. In some embodiments, the first antigen binding domain comprises the V_{H}EpCAM and the V_{L}EpCAM selected from the group consisting of: (a) the V_{H}EpCAM comprising the amino acid sequence of SEQ ID NO:27, and the V_{L}EpCAM comprising the amino acid sequence of SEQ ID NO:29; (b) the V_{H}EpCAM comprising the amino acid sequence of SEQ ID NO:31, and the V_{L}EpCAM comprising the amino acid of SEQ ID NO:33; and (c) the V_{H}EpCAM comprising the amino acid sequence of SEQ ID NO:35, and the V_{L}EpCAM comprising the amino acid sequence of SEQ ID NO:37.

In some embodiments, the second antigen binding domain specifically binds to CD3ε, CD3γ, or CD3δ chain. In some embodiments, the second antigen binding domain comprises V_{H}CD3 and V_{L}CD3, wherein the V_{H}CD3 comprises the amino acid sequence of SEQ ID NO:55, and/or wherein the V_{L}CD3 comprises the amino acid sequence of SEQ ID NO:57. In some embodiments, the second antigen binding domain comprises V_{H}CD3 and V_{L}CD3, wherein the V_{H}CD3 comprises the three CDRs from the amino acid sequence of SEQ ID NO:55, and/or wherein the V_{L}CD3 comprises the three CDRs from the amino acid sequence of SEQ ID NO:57. In some embodiments, the V_{H}CD3 and/or V_{L}CD3 are humanized.

In some embodiments, the bispecific antibody further comprises a human serum albumin sequence (HSA) at the C-terminus of the bispecific antibody. In some embodiments, the human serum albumin sequence comprising the amino acid sequence of SEQ ID NO: 45 or SEQ ID NO:47. In some embodiments, the bispecific antibody further comprises a peptide linker between the V_{L}CD3 and the human serum albumin sequence. In some embodiments, the peptide linker between the V_{L}CD3 and the human serum albumin sequence comprises the amino acid sequence of SEQ ID NO:51.

In some embodiments, the bispecific antibody comprises the amino acid sequence selected from the group consisting of SEQ ID NO:39, and SEQ ID NO:41, and SEQ ID NO:43.

The invention also provides pharmaceutical compositions comprising an antibody described herein and a pharmaceutically acceptable carrier. In some embodiments, the antibody is an anti-EpCAM antibody described herein. In some embodiments, the antibody is a single-chain bispecific antibody described in.

The invention also provides an isolated polynucleotide comprising one or more nucleic acid sequences encoding an antibody described herein (e.g., an anti-EpCAM antibody). In some embodiments, the polynucleotide comprises one or more amino acid sequences encoding a single-chain bispecific antibody described in. The invention also provides a vector comprising a polynucleotide described herein. The invention also provides a host cell comprising a vector described herein.

The invention also provides methods of producing an antibody described herein (e.g., an anti-EpCAM antibody), comprising culturing a host cell described herein that produces the antibody, and recovering the antibody from the cell culture. In some embodiments, the antibody is a single-chain bispecific antibody described in. In some embodiments, the host cells comprise a vector comprising one or more nucleic acid sequences encoding the antibody.

The invention also provides methods of screening an antibody that specifically binds human EpCAM and induces apoptosis of human cancer cells in vitro, comprising: (a) culturing a cancer cell with an effective concentration of a naked monoclonal antibody that specifically binds to human EpCAM in vitro; (b) measuring the apoptosis of the cancer cell induced by the naked monoclonal antibody; and (c) selecting the antibody if the antibody has higher apoptosis-inducing activity as compared to a control antibody. In some embodiments, the antibody is an anti-EpCAM antibody described herein. In some embodiments, the antibody is a single-chain bispecific antibody described in. In some embodiments, the apoptosis-inducing activity is measured by Annexin V and Propidium Iodide staining of the cancer cell. In some embodiments, the cancer cell is selected from the group consisting of a breast cancer cell, a colorectal cancer cell, a gastric cancer cell, a lung cancer cell, a prostate cancer cell, a pancreatic cancer cell, a pharynx cancer cell, and an ovarian cancer cell. In some embodiments, the control antibody is an antibody selected from the group consisting of 12H8, 1F10, 1G10, 2D11, 6D11, and 4D2. In some embodiments, an antibody having at least 90% of the apoptosis-inducing activity as an antibody selected from the group consisting of 12H8, 1F10, 1G10, 2D11, 6D11, and 4D2 is selected.

The invention also provides methods for treating, delaying development, and/or preventing a cancer in an individual comprising administering to the individual an effective amount of an antibody described herein. In some embodiments, an anti-EpCAM antibody described herein is used. In some embodiments, a single-chain bispecific antibody described in is used. In some embodiments, the cancer is selected from the group consisting of breast cancer, colorectal cancer, gastric cancer, lung cancer, prostate cancer, pancreatic cancer, pharynx cancer, and ovarian cancer. In some embodiments, the individual is selected for treatment by detecting binding of the antibody to the cancer cells in the individual. In some embodiments, the methods further comprise administering to the individual a second anti-cancer agent. In some embodiments, the second anti-cancer agent is a chemotherapeutic agent (such as Oxaliplatin).

The invention also provides kits comprising an antibody described herein. In some embodiments, the kit comprises an anti-EpCAM antibody described herein. In some embodiments, the kit comprises a single-chain bispecific antibody described in. In some embodiments, the kits may further comprise instructions for administering an effective amount of the antibody to an individual for treating cancer in the individual. In some embodiments, the kits may further comprise a second anti-cancer agent and/or instructions for administering the antibody and the second anti-cancer agent in conjunction to an individual for treating cancer in the individual.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 shows the amino acid sequence of human EpCAM (SEQ ID NO:1).

FIG. 2 shows the amino acid sequence of the EGF-I domain (amino acids 24-63) (SEQ ID NO:2) of human EpCAM and amino acid substitutions in the mutant clones.

FIG. 3 shows the sequences of murine antibody 12H8 variable regions. FIG. 3A shows the amino acid (SEQ ID NO:3) and nucleic acid (SEQ ID NO:4) sequence of the heavy chain variable region, and FIG. 3B shows the amino acid (SEQ ID NO:5) and nucleic acid (SEQ ID NO:6) sequence of the light chain variable region. CDR sequences are underlined.

FIG. 4 shows the sequences of murine antibody 1G10 variable regions. FIG. 4A shows the amino acid (SEQ ID NO:7) and nucleic acid (SEQ ID NO:8) sequence of the heavy chain variable region, and FIG. 4B shows the amino acid (SEQ ID NO:9) and nucleic acid (SEQ ID NO:10) sequence of the light chain variable region. CDR sequences are underlined.

FIG. 5 shows the sequences of murine antibody 1F10 variable regions. FIG. 5A shows the amino acid (SEQ ID NO:11) and nucleic acid (SEQ ID NO:12) sequence of the heavy chain variable region, and FIG. 5B shows the amino acid (SEQ ID NO:13) and nucleic acid (SEQ ID NO:14) sequence of the light chain variable region. CDR sequences are underlined.

FIG. 6 shows the sequences of murine antibody 2D11 variable regions. FIG. 6A shows the amino acid (SEQ ID NO:15) and nucleic acid (SEQ ID NO:16) sequence of the heavy chain variable region, and FIG. 6B shows the amino acid (SEQ ID NO:17) and nucleic acid (SEQ ID NO:18) sequence of the light chain variable. CDR sequences are underlined.

FIG. 7 shows the sequences of murine antibody 4D2 variable regions. FIG. 7A shows the amino acid (SEQ ID NO:19) and nucleic acid (SEQ ID NO:20) sequence of the heavy chain variable region, and FIG. 7B shows the amino acid (SEQ ID NO:21) and nucleic acid (SEQ ID NO:22) sequence of the light chain variable region. CDR sequences are underlined.

FIG. 8 shows the sequences of murine antibody 6D11 variable regions. FIG. 8A shows the amino acid (SEQ ID NO:23) and nucleic acid (SEQ ID NO:24) sequence of the heavy chain variable region, and FIG. 8B shows the amino acid (SEQ ID NO:25) and nucleic acid (SEQ ID NO:26) sequence of the light chain variable region. CDR sequences are underlined.

FIGS. 9A and 9B show the construction of anti-EpCAM and anti-CD3 bispecific antibodies without HSA fusion (FIG. 9A) and with HSA fusion (FIG. 9B).

FIG. 10A shows the sequence for h12H8B V_{L} (SEQ ID NO:29 for amino acid sequence; SEQ ID NO:30 for nucleic acid sequence). FIG. 10B shows the sequence for h12H8B V_{H} (SEQ ID NO:27 for amino acid sequence; SEQ ID NO:28 for nucleic acid sequence).

FIG. 11A shows the sequence for h12H8C V_{L} (SEQ ID NO:33 for amino acid sequence; SEQ ID NO:34 for nucleic acid sequence). FIG. 11B shows the sequence for h12H8C V_{H} (SEQ ID NO:31 for amino acid sequence; SEQ ID NO:32 for nucleic acid sequence).

FIG. 12A shows the sequence for h2D11B V_{L} (SEQ ID NO:37 for amino acid sequence; SEQ ID NO:38 for nucleic acid sequence). FIG. 12B shows the sequence for h2D11B V_{H} (SEQ ID NO:35 for amino acid sequence; SEQ ID NO:36 for nucleic acid sequence).

FIG. 13A shows the sequence for anti-CD3 V_{L} (SEQ ID NO:57 for amino acid sequence; SEQ ID NO:58 for nucleic acid sequence). FIG. 13B shows the sequence for anti-CD3 V_{H} (SEQ ID NO:55 for amino acid sequence; SEQ ID NO:56 for nucleic acid sequence).

FIG. 14 shows sequences for v1 version of anti-EpCAM x anti-CD3 bsAbs. FIG. 14A: the sequence for v1 version h12H8B bsAb (“h12H8B-v1”) (SEQ ID NO:39 for amino acid sequence; SEQ ID NO:40 for nucleic acid sequence). FIG. 14B: the sequence for v1 version h12H8C bsAb (“h12H8C-v1”) (SEQ ID NO:41 for amino acid sequence; SEQ ID NO:42 for nucleic acid sequence). FIG. 14C: the sequence for v1 version h2D11B bsAb (“h2D11B-v1”) (SEQ ID NO:43 for amino acid sequence; SEQ ID NO:44 for nucleic acid sequence).

FIG. 15A shows the sequence for full-length albumin (SEQ ID NO:45 for amino acid sequence; SEQ ID NO:46 for nucleic acid sequence) used for bsAb fusion. FIG. 15B shows the sequence for short-form albumin (SEQ ID NO:47 for amino acid sequence; SEQ ID NO:48 for nucleic acid sequence) used for bsAb fusion.

FIGS. 16A and 16B show effect of AbGn bsAbs on cytotoxic activity (% of cell death) in pancreatic cancer cell Panc 02.03 (FIG. 16A) and Multiple Myeloma cell RPMI 8266 (FIG. 16B) in the presence of human PBMC.

FIGS. 17A and 17B show effect of bsAbs on % of cell growth inhibition in pancreatic cancer cell Panc 02.03 (FIG. 17A) and lung cancer cell NCI-H358 (FIG. 17B) in the presence of hPBMC.

FIG. 18 shows antitumor activity of h12H8CXanti-CD3 (h12H8C-v2.1 and h12H8C-v2.1-sHSA) in human DLD-1 colon carcinoma xenograft model (Mean±SEM) (*p<0.05 as compared to PBS control group).

### DETAILED DESCRIPTION OF THE INVENTION

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibody of the present invention is further intended to include bispecific, multispecific, single-chain, and chimeric and humanized molecules having affinity for a polypeptide conferred by at least one CDR region of the antibody. Antibodies of the present invention also include single domain antibodies which are either the variable domain of an antibody heavy chain or the variable domain of an antibody light chain. Holt et al., (2003), Trends Biotechnol. 21:484-490. Methods of making domain antibodies comprising either the variable domain of an antibody heavy chain or the variable domain of an antibody light chain, containing three of the six naturally occurring complementarity determining regions from an antibody, are also known in the art. See, e.g., Muyldermans, Rev. Mol. Biotechnol. 74:277-302, 2001.

As used herein, “monoclonal antibody” refers to an antibody of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), monoclonal antibody is not a mixture of discrete antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, (1975), Nature, 256:495, or may be made by recombinant DNA methods such as described in U.S. Pat. No. 4,816,567. The monoclonal antibodies may also be isolated from phage libraries generated using the techniques described in McCafferty et al., (1990), Nature, 348:552-554, for example. It should be understood that a monoclonal antibody used herein includes chimeric antibodies, humanized antibodies, human antibodies, single-chain antibodies, single-domain antibodies, bispecific antibodies, multispecific antibodies, and antigen-binding fragments thereof.

As used herein, a “chimeric antibody” refers to an antibody having a variable region or part of variable region from a first species and a constant region from a second species. An intact chimeric antibody comprises two copies of a chimeric light chain and two copies of a chimeric heavy chain. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammals, while the constant portions are homologous to the sequences in antibodies derived from another. In some embodiments, amino acid modifications can be made in the variable region and/or the constant region.

An “isolated” antibody is one which has been identified and separated and/or recovered from a component of its natural environment.

As used herein, a “naked antibody” is an antibody that is not conjugated to a cytotoxic moiety or radiolabel.

As used herein, “substantially pure” refers to material which is at least 50% pure (i.e., free from contaminants), more preferably at least 90% pure, more preferably at least 95% pure, more preferably at least 98% pure, more preferably at least 99% pure.

As used herein, “humanized” antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., (1998), PNAS, (USA) 95:6157-6162; Hoogenboom and Winter, 1991, J. Mol. Biol., 227:381; Marks et al., (1991), J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., (1991), J. Immunol., 147 (1):86-95; and U.S. Pat. No. 5,750,373.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination. A constant region of an antibody generally provides structural stability and other biological functions such as antibody chain association, secretion, transplacental mobility, and complement binding, but is not involved with binding to the antigen. The amino acid sequence and corresponding exon sequences in the genes of the constant region will be dependent upon the species from which it is derived; however, variations in the amino acid sequence leading to allotypes will be relatively limited for particular constant regions within a species. The variable region of each chain is joined to the constant region by a linking polypeptide sequence. The linkage sequence is coded by a “J” sequence in the light chain gene, and a combination of a “D” sequence and a “J” sequence in the heavy chain gene.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “individual” or a “subject” is a mammal, more preferably a human. Mammals also include, but are not limited to, farm animals, sport animals, pets (such as cats, dogs, horses), primates, mice and rats.

As use herein, the term “specifically recognizes” or “specifically binds” refers to measurable and reproducible interactions such as attraction or binding between a target and an antibody, that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³M⁻¹ or 10⁴M⁻¹, sometimes about 10⁵M⁻¹ or 10⁶M⁻¹, in other instances about 10⁶M⁻¹ or 10⁷M⁻¹, about 10⁸M⁻¹ to 10⁹M⁻¹, or about 10¹⁰M⁻¹ to 10¹¹M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “cancer,” “tumor,” “cancerous,” and “malignant” refer to or describe the physiological condition in mammals that is typically characterized by unregulated cell growth. Examples of cancer include but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, prostate cancer, thyroid cancer, testicular cancer, esophageal cancer, and various types of head and neck cancer.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

Antibodies and Polypeptides that Specifically Bind to EpCAM

The invention provides isolated antibodies, and polypeptides derived from the antibodies, that specifically bind to EpCAM expressed on the cell surface of the cancer cells and induce substantial apoptosis of the cancer cells in vitro in the absence of cytotoxin conjugation, immune effector functions (includes ADCC and CDC activity), and any agents that cross-link the antibodies.

The invention provides an isolated monoclonal antibody, which antibody specifically binds to an epitope within amino acids 24-63 of human EpCAM, wherein the naked antibody induces apoptosis of human cancer cells after binding to the epitope on the cell surface of the cancer cells in vitro. In some embodiments, the apoptosis-inducing activity of the naked antibody to human lung cancer cell line NCI-H358 is at least about 90% of an antibody selected from the group consisting of 12H8, 1F10, 1G10, 2D11, 6D11, and 4D2 wherein the apoptosis-inducing activity is measured by incubating the human lung cancer cell line with an antibody at concentration of about 10 ug/ml and an incubation time of about 16-20 hours.

In some embodiments, the antibodies or the polypeptides of the invention bind to human EpCAM shown in FIG. 1 and/or a naturally occurring variants.

In some embodiments, the antibodies or polypeptides of the invention bind to the EpCAM expressed on the cell surface of cancer cells and the naked antibodies or polypeptides induce apoptosis of the cancer cells in vitro. In some embodiments, the apoptosis-inducing activity of the antibodies or polypeptides is at least about 90% of the activity of an antibody selected from the group consisting of 12H8, 1F10, 1G10, 2D11, 6D11, and 4D2, wherein the apoptosis-inducing activity is measured by incubating human lung cancer cell NCI-H358 at an antibody or polypeptide concentration of about 10 ug/ml and an incubation time of about 16-20 hours. In some embodiments, the apoptosis-inducing activity of the antibodies or polypeptides is at least about 95%, at least about 100%, or higher of the activity of an antibody selected from the group consisting of 12H8, 1F10, 1G10, 2D11, 6D11, and 4D2. Human lung cancer cell line NCI-H358 is from bronchioalveolar carcinoma, and can be obtained from ATCC at ATCC accession No. PTA-10386.

The antibodies and polypeptides of the invention are capable of inducing apoptosis alone after binding the EpCAM expressed on the cell surface of the cancer cells. The term “inducing apoptosis” as used herein, means that the antibodies or polypeptides of the present invention, can directly interact with a molecule expressed on the cell surface, and the binding/interaction alone is sufficient to induce apoptosis in the cells without the help of other factors such as cytotoxin conjugation, other immune effector functions (i.e., complement-dependent cytotoxicity (CDC), antibody-dependent cellular cytotoxicity (ADCC), or phagocytosis), or an cross-linking agent.

As used herein, the term “apoptosis” refers to gene-directed process of intracellular cell destruction. Apoptosis is distinct from necrosis; it includes cytoskeletal disruption, cytoplasmic shrinkage and condensation, expression of phosphatidylserine on the outer surface of the cell membrane and blebbing, resulting in the formation of cell membrane bound vesicles or apoptotic bodies. The process is also referred to as “programmed cell death.” During apoptosis, characteristic phenomena such as curved cell surfaces, condensation of nuclear chromatin, fragmentation of chromosomal DNA, and loss of mitochondrial function are observed. Various known technologies may be used to detect apoptosis, such as staining cells with Annexin V, propidium iodide, DNA fragmentation assay and YO-PRO-1 (Invitrogen). In some embodiments, staining with Annexin V and propidium iodide may be used, and the combined percentages of the Annexin V+/PI+, Annexin V+/PI− and Annexin V−/PI+ populations are considered as dead cells.

In some embodiments, the antibodies or polypeptides of the invention binds to an epitope within amino acids 24-63 of SEQ ID NO:1. The binding may specifically depend on the presence of specific amino acid residues within the region. For example, binding the antibody or polypeptide may depend on the presence of following groups of amino acid residues: (1) Q24, E25, and N42; (2) Q24, E25, E26, N37, N41, Q47, and T49; (3) E25, V40, and R44; (4) N41, N43, and R44; or (5) Q24, E25, A35, F39, V40, N41, R44, Q45, and Q47. To determine if binding of an antibody depends on the presence of an amino acid residue, relative binding activities of the antibody to a mutant EpCAM may be compared to wild-type EpCAM as described in Example 6. If an amino acid mutation to an unrelated amino acid (such as mutated to a corresponding murine EpCAM amino acid residue or a non-conservative mutation) causes more than 50% reduction in the relative binding activity, it is considered that the binding of the antibody depends on the presence of the amino acid residue.

In some embodiments, the invention provides monoclonal antibodies that compete with any of the antibody selected from the group consisting of 12H8, 1F10, 1G10, 2D11, 6D11, and 4D2 for binding to EpCAM. In some embodiments, the invention provides monoclonal antibodies that bind to the same epitope as any of the antibody selected from the group consisting of 12H8, 1F10, 1G10, 2D11, 6D11, and 4D2.

Competition assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels. For example, immobilized EpCAM is incubated with a first labeled antibody that binds to EpCAM and an increasing concentrations of a second unlabeled antibody. As a control, immobilized EpCAM is incubated with the first labeled antibody without the second unlabeled antibody. After incubation under conditions that allow binding the first antibody to EpCAM, excess unbound antibody is removed and the amount of label bound to the immobilized EpCAM is measured. If the amount of label bound to the immobilized EpCAM is substantially reduced (for example, reduced at least about 50%, at least about 60%, at least about 70%, at least about 80%, or at least about 90%) in the test sample relative to the control sample when the concentration of the second unlabeled antibody to the first labeled antibody in the test is 100:1 or higher (such as 500:1 or higher, or 1000:1 or higher), the second antibody is considered as competing with the first antibody for binding to EpCAM. Other methods may be used to for mapping to which an antibody binds are provided in Morris (1996) “Epitope Mapping Protocols,” in Methods in Molecular Biology v. 66 (Humana Press, Totowa, N.J.).

In some embodiments, the invention provides an anti-EpCAM antibody comprising: a heavy chain variable region comprising one, two or the three CDRs of SEQ ID NO:3 and/or a light chain variable region comprising one, two, or the three CDRs of SEQ ID NO:5. In some embodiments, the invention provides an anti-EpCAM antibody comprising: a heavy chain variable region comprising one, two or the three CDRs of SEQ ID NO:7 and/or a light chain variable region comprising one, two, or the three CDRs of SEQ ID NO:9. In some embodiments, the invention provides an anti-EpCAM antibody comprising: a heavy chain variable region comprising one, two or the three CDRs of SEQ ID NO:11 and/or a light chain variable region comprising one, two, or the three CDRs of SEQ ID NO:13. In some embodiments, the invention provides an anti-EpCAM antibody comprising: a heavy chain variable region comprising one, two or the three CDRs of SEQ ID NO:15 and/or a light chain variable region comprising one, two, or the three CDRs of SEQ ID NO:17. In some embodiments, the invention provides an anti-EpCAM antibody comprising: a heavy chain variable region comprising one, two or the three CDRs of SEQ ID NO:19 and/or a light chain variable region comprising one, two, or the three CDRs of SEQ ID NO:21. In some embodiments, the invention provides an anti-EpCAM antibody comprising: a heavy chain variable region comprising one, two or the three CDRs of SEQ ID NO:23 and/or a light chain variable region comprising one, two, or the three CDRs of SEQ ID NO:25.

In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined.

In some embodiments, anti-EpCAM antibodies described herein may have one or more CDRs having amino acid sequences that are at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or six CDRs of the antibody selected from the group consisting of 12H8, 1F10, 1G10, 2D11, 6D11, and 4D2.

In some embodiments, the invention provides an anti-EpCAM antibody comprising a heavy chain variable region comprising the amino acid sequence of SEQ ID NO:3 and/or a light chain variable region comprising the amino acid sequence of SEQ ID NO:5. In some embodiments, the invention provides an anti-EpCAM antibody comprising a heavy chain variable region comprising the amino acid sequence of SEQ ID NO:7 and/or a light chain variable region comprising the amino acid sequence of SEQ ID NO:9. In some embodiments, the invention provides an anti-EpCAM antibody comprising a heavy chain variable region comprising the amino acid sequence of SEQ ID NO:11 and/or a light chain variable region comprising the amino acid sequence of SEQ ID NO:13. In some embodiments, the invention provides an anti-EpCAM antibody comprising a heavy chain variable region comprising the amino acid sequence of SEQ ID NO:15 and/or a light chain variable region comprising the amino acid sequence of SEQ ID NO:17. In some embodiments, the invention provides an anti-EpCAM antibody comprising a heavy chain variable region comprising the amino acid sequence of SEQ ID NO:19 and/or a light chain variable region comprising the amino acid sequence of SEQ ID NO:21. In some embodiments, the invention provides an anti-EpCAM antibody comprising a heavy chain variable region comprising the amino acid sequence of SEQ ID NO:23 and/or a light chain variable region comprising the amino acid sequence of SEQ ID NO:25.

In some embodiments, one or more amino acid residues in the heavy chain constant region and/or the light chain constant region of the antibody are modified (including amino acid insertion, deletion, and substitution). The modified amino acid sequence is at least about 90%, at least about 91%, at least about 92%, at least about 93%, at least about 94%, at least about 95%, at least about 96%, at least about 97%, at least about 98%, or at least about 99% identical to the sequence before modification.

Examples of cancer cells expressing EpCAM include, but are not limited to, breast cancer, colorectal cancer, gastric cancer, lung cancer, prostate cancer, pancreatic cancer, pharynx cancer and ovarian cancer.

The invention encompasses modifications to antibodies or polypeptide described herein, including functionally equivalent antibodies which do not significantly affect their properties and variants which have enhanced or decreased activity and/or affinity. For example, amino acid sequence of antibody may be mutated to obtain an antibody with the desired binding affinity to the EpCAM expressed by the cancer cell. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in the table below under the heading of “conservative substitutions”. If such substitutions result in a change in biological activity, then more substantial changes, denominated “exemplary substitutions” in the table below, or as further described below in reference to amino acid classes, may be introduced and the products screened.

TABLE 1
Amino Acid Substitutions.

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Non-polar: Norleucine, Met, Ala, Val, Leu, Ile;

(2) Polar without charge: Cys, Ser, Thr, Asn, Gln;

(3) Acidic (negatively charged): Asp, Glu;

(4) Basic (positively charged): Lys, Arg;

(5) Residues that influence chain orientation: Gly, Pro; and

(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDRH3 and/or CDR L3.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, (1997), Chem. Immunol. 65:111-128; Wright and Morrison, (1997), TibTECH 15:26-32). The oligosaccharide side chains of the immunoglobulins affect the protein's function (Boyd et al., (1996), Mol. Immunol. 32:1311-1318; Wittwe and Howard, (1990), Biochem. 29:4175-4180) and the intramolecular interaction between portions of the glycoprotein, which can affect the conformation and presented three-dimensional surface of the glycoprotein (Hefferis and Lund, supra; Wyss and Wagner, (1996), Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., (1999), Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., (1997), J. Biol. Chem. 272:9062-9070).

It may be desirable to modify the Fc region of the antibodies described herein with respect to effector functions, e.g., enhancing antigen-dependent cell-mediated cytotoxicity (ADCC) and/or complement dependent cytotoxicity (CDC) of the antibody. This may be achieved by introducing one or more amino acid substitutions in the Fc region. The amino acids that may be substituted are known in the art.

The antibodies of the invention can encompass antibody fragments (e.g., Fab, Fab′, F(ab′)₂, Fv, Fc, etc.), chimeric antibodies, single chain (ScFv), mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies).

The binding affinity of the polypeptide (including antibody) to EpCAM may be less than any of about 500 nM, about 400 nM, about 300 nM, about 200 nM, about 100 nM, about 50 nM, about 10 nM, about 1 nM, about 500 pM, about 100 pM, or about 50 pM. As is well known in the art, binding affinity can be expressed as K_{D}, or dissociation constant, and an increased binding affinity corresponds to a decreased K_{D}. One way of determining binding affinity of antibodies to EpCAM is by measuring binding affinity of monofunctional Fab fragments of the antibody. To obtain monofunctional Fab fragments, an antibody (for example, IgG) can be cleaved with papain or expressed recombinantly. The affinity of a Fab fragment of an antibody can be determined by surface plasmon resonance (BIAcore3000™ surface plasmon resonance (SPR) system, BIAcore, INC, Piscaway N.J.) and ELISA. Kinetic association rates (kₒₙ) and dissociation rates (k_{off}) (generally measured at 25° C.) are obtained; and equilibrium dissociation constant (K_{D}) values are calculated as k_{off}/kₒₙ.

Methods of making antibodies and polypeptides derived from the antibodies are known in the art and are disclosed herein. Antibodies generated may be tested for having specific binding to an epitope within amino acids 24-63 of human EpCAM expressed by the cancer cells.

The binding specificity of the antibodies produced may be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220.

The antibodies identified may further be tested for their capabilities to induce cell death (e.g., apoptosis), and/or inhibiting cell growth or proliferation using methods known in the art and described herein.

The invention also provides methods for screening an antibody that specifically binds human EpCAM and induces apoptosis of human cancer cells in vitro, comprising: (a) culturing a cancer cell with an effective concentration of a naked monoclonal antibody that specifically binds to human EpCAM in vitro; (b) measuring the apoptosis of the cancer cell induced by the naked monoclonal antibody; and (c) selecting the antibody if the antibody has higher apoptosis-inducing activity as compared to a control antibody. Any methods described herein or known in the art can be used for measuring apoptosis inducing activity of an antibody. In some embodiments, the antibody having at least 80%, at least 90%, at least 95% of the apoptosis-inducing activity as the antibody selected from the group consisting of 12H8, 1F10, 1G10, 2D11, 6D11, and 4D2 is selected. In some embodiments, the human cancer cells are breast cancer cells, colorectal cancer cells, gastric cancer cells, lung cancer cells, prostate cancer cells, pancreatic cancer cells, pharynx cancer cells, and ovarian cancer cells. Methods for screening anti-EpCAM antibodies having apoptosis-inducing activity are described in the detail in Example 3.

The antibodies of the invention can also be made by recombinant DNA methods, such as those described in U.S. Pat. Nos. 4,816,567 and 6,331,415, which are hereby incorporated by reference. For example, DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiment, the antibodies of the present invention are expressed from two expression vectors. In some embodiments, the first expression vector encodes a heavy chain of the antibody (e.g., a humanized antibody), comprising a first part encoding a variable region of the heavy chain of the antibody, and a second part encoding a constant region of the heavy chain of the antibody. In some embodiments, the second expression vector encodes a light chain of the antibody, comprising a first part encoding a variable region of the light chain of the antibody, and a second part encoding a constant region of the light chain of the antibody.

Alternatively, the antibodies (e.g., a humanized antibody) of the present invention are expressed from a single expression vector. The single expression vector encodes both the heavy chain and light chain of the antibodies of the present invention.

Normally the expression vector has transcriptional and translational regulatory sequences which are derived from species compatible with a host cell. In addition, the vector ordinarily carries a specific gene(s) which is (are) capable of providing phenotypic selection in transformed cells.

A wide variety of recombinant host-vector expression systems for eukaryotic cells are known and can be used in the invention. For example, Saccharomyces cerevisiae, or common baker's yeast, is the most commonly used among eukaryotic microorganisms, although a number of other strains, such as Pichia pastoris, are available. Cell lines derived from multicellular organisms such as Sp2/0 or Chinese Hamster Ovary (CHO), which are available from the ATCC, may also be used as hosts. Typical vector plasmids suitable for eukaryotic cell transformations are, for example, pSV2neo and pSV2gpt (ATCC), pSVL and pSVK3 (Pharmacia), and pBPV-1/pML2d (International Biotechnology, Inc.).

The eukaryotic host cells useful in the present invention are, preferably, hybridoma, myeloma, plasmacytoma or lymphoma cells. However, other eukaryotic host cells may be suitably utilized provided the mammalian host cells are capable of recognizing transcriptional and translational DNA sequences for expression of the proteins; processing the leader peptide by cleavage of the leader sequence and secretion of the proteins; and providing post-translational modifications of the proteins, e.g., glycosylation.

Accordingly, the present invention provides eukaryotic host cells which are transformed by recombinant expression vectors comprising DNA constructs disclosed herein and which are capable of expressing the antibodies or polypeptides of the present invention. In some embodiments, the transformed host cells of the invention, therefore, comprise at least one DNA construct comprising the light and heavy chain DNA sequences described herein, and transcriptional and translational regulatory sequences which are positioned in relation to the light and heavy chain-encoding DNA sequences to direct expression of antibodies or polypeptides.

The host cells used in the invention may be transformed in a variety of ways by standard transfection procedures well known in the art. Among the standard transfection procedures which may be used are electroporation techniques, protoplast fusion and calcium-phosphate precipitation techniques. Such techniques are generally described by F. Toneguzzo et al. (1986), Mol. Cell. Biol., 6:703-706; G. Chu et al., Nucleic Acid Res. (1987), 15:1311-1325; D. Rice et al., Proc. Natl. Acad. Sci. USA (1979), 79:7862-7865; and V. Oi et al., Proc. Natl. Acad. Sci. USA (1983), 80:825-829.

In the case of two expression vectors, the two expression vectors can be transferred into a host cell one by one separately or together (co-transfer or co-transfect).

The present invention also provides a method for producing the antibodies or polypeptides, which comprises culturing a host cell comprising an expression vector(s) encoding the antibodies or the polypeptides, and recovering the antibodies or polypeptides from the culture by ways well known to one skilled in the art. In some embodiments, the antibodies may be isolated or purified by conventional immunoglobulin purification procedures such as, for example, protein A-Sepharose, hydroxylapatite chromatography, gel electrophoresis, dialysis, or affinity chromatography.

Furthermore, the desired antibodies can be produced in a transgenic animal. A suitable transgenic animal can be obtained according to standard methods which include micro-injecting into eggs the appropriate expression vectors, transferring the eggs into pseudo-pregnant females and selecting a descendant expressing the desired antibody.

The present invention also provides chimeric antibodies that specifically recognize EpCAM. For example, the variable and constant regions of the chimeric antibody are from separate species. In some embodiments, the variable regions of both heavy chain and light chain are from the murine antibodies described herein. In some embodiments, the variable regions comprise amino acid sequences from variable regions from SEQ ID NO: 3, 5, 7, 9, 11, 13, 15, 17, 19, 21, 23, or 25. In some embodiments, the constant regions of both the heavy chain and light chain are from human antibodies.

The chimeric antibody of the present invention can be prepared by techniques well-established in the art. See for example, U.S. Pat. No. 6,808,901, U.S. Pat. No. 6,652,852, U.S. Pat. No. 6,329,508, U.S. Pat. No. 6,120,767 and U.S. Pat. No. 5,677,427, each of which is hereby incorporated by reference. In general, the chimeric antibody can be prepared by obtaining cDNAs encoding the heavy and light chain variable regions of the antibodies, inserting the cDNAs into an expression vector, which upon being introduced into eukaryotic host cells, expresses the chimeric antibody of the present invention. Preferably, the expression vector carries a functionally complete constant heavy or light chain sequence so that any variable heavy or light chain sequence can be easily inserted into the expression vector.

The present invention provides a humanized antibody that specifically recognizes EpCAM. The humanized antibody is typically a human antibody in which residues from CDRs are replaced with residues from CDRs of a non-human species such as mouse, rat or rabbit having the desired specificity, affinity and capacity. In some instances, Fv framework residues of the human antibody are replaced by corresponding non-human residues.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

It is important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. The humanized antibodies may also contain modifications in the hinge region to improve one or more characteristics of the antibody.

In another alternative, antibodies may be screened and made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines and bovines.

In certain embodiments, the antibody is a fully human antibody. Non-human antibodies that specifically bind an antigen can be used to produce a fully human antibody that binds to that antigen. For example, the skilled artisan can employ a chain swapping technique, in which the heavy chain of a non-human antibody is co-expressed with an expression library expressing different human light chains. The resulting hybrid antibodies, containing one human light chain and one non-human heavy chain, are then screened for antigen binding. The light chains that participate in antigen binding are then co-expressed with a library of human antibody heavy chains. The resulting human antibodies are screened once more for antigen binding. Techniques such as this one are further described in U.S. Pat. No. 5,565,332. In addition, an antigen can be used to inoculate an animal that is transgenic for human immunoglobulin genes. See, e.g., U.S. Pat. No. 5,661,016.

The antibody may be a bispecific antibody, a monoclonal antibody that has binding specificities for at least two different antigens (including epitopes). The invention provides a bispecific antibody comprising a first binding domain that specifically recognize a human EpCAM and a second binding domain that specifically recognize a different antigen. In some embodiments, the second binding domain in the bispecific antibody specifically recognizes a CD3 (e.g., human CD3). In some embodiments, the bispecific antibody comprises a heavy chain variable region comprising one, two, or three CDRs derived from the heavy chain of any of the anti-EpCAM antibodies described herein (e.g., 12H8, 1G10, 1F10, 2D11, 6D11, 4D2) and/or a light chain variable region comprising one, two, or three CDRs derived from the light chain of any of the anti-EpCAM antibodies described herein (e.g., 12H8, 1G10, 1F10, 2D11, 6D11, 4D2).

Bispecific antibodies that bind to both EpCAM and CD3 can be made using methods known in the art. For example, there are two bispecific antibodies with EpCAM and CD3 combination are currently being tested in clinical trials. Catumaxomab is a trifunctional designed of bispecific antibody in clinical development for the treatment of patients with malignant ascites (Fresenius Biotech & Trion, Clin Cancer Res 2007; 13(13):3899, British Journal of Cancer 2007; 97:315-321, Journal of Experimental & Clinical Cancer Research 2009; 28:18, J Clin Oncol 2009; 27:15s (suppl; abstr 3036)). MT110 is the other class of bispecific T-cell engaging (BiTE) antibody that is using single-chain Fv (scFv) construct. MT110 is currently being tested in a phase 1 trial with lung and gastrointestinal cancer patients (Micromet Inc., Bethesda, Cancer Res 2009; 69(12):4941-4, Molecular Immunology 2006; 43:1129-1143, Cancer Res 2008; 68(1):143-51, Cancer Res 2009; 69(12):4941-4, Immunobiology 2009; 214:441-453).

Methods for making bispecific antibodies are known in the art (see, e.g., Suresh et al., (1986), Methods in Enzymology 121:210). Traditionally, the recombinant production of bispecific antibodies was based on the coexpression of two immunoglobulin heavy chain-light chain pairs, with the two heavy chains having different specificities (Millstein and Cuello, (1983), Nature 305, 537-539). In some embodiments, the bispecific antibodies specifically bind to both EpCAM and CD3.

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

The invention provides single-chain bispecific antibodies, for example, single-chain bispecific antibodies comprising (a) a first antigen binding domain that specifically binds to human EpCAM and (b) a second antigen binding domain that specifically binds to human CD3 antigen. The first antigen binding domain specifically binds to human EpCAM, for example, it specifically binds to an epitope within amino acids 24-63 of human EpCAM. In some embodiments, the first antigen binding domain comprises a heavy chain variable region (V_{H}EpCAM) and/or a light chain variable region (V_{L}EpCAM). The second antigen binding domain specifically binds to human CD3 antigen. In some embodiments, the second antigen binding domain comprises a heavy chain variable region (V_{H}CD3) and/or a light chain variable region (V_{L}CD3). The variable regions can be arranged from N-terminus to C-terminus in an order that allows a bispecific antibody provided herein to specifically binds to human EpCAM and specifically binds to human CD3 antigen, for example, the variable regions can be arranged from N-terminus to C-terminus in the order such as V_{L}EpCAM-V_{H}EpCAM-V_{H}CD3-V_{L}CD3 or V_{L}CD3-V_{H}CD3-V_{H}EpCAM-V_{L}EpCAM. In some embodiments, the invention provides a single-chain bispecific antibody comprising (a) a first antigen binding domain that specifically binds to an epitope within amino acids 24-63 of human EpCAM, wherein the first antigen binding domain comprises a heavy chain variable region (V_{H}EpCAM) and a light chain variable region (V_{L}EpCAM); and (b) a second antigen binding domain that specifically binds to human CD3 antigen, wherein the second antigen binding domain comprises a heavy chain variable region (V_{H}CD3) and a light chain variable region (V_{L}CD3); wherein the variable regions are arranged from N-terminus to C-terminus in the order V_{L}EpCAM-V_{H}EpCAM-V_{H}CD3-V_{L}CD3. “V_{L}EpCAM” and “V_{H}EpCAM” mean the light chain and heavy chain, respectively, of the variable region of anti-EpCAM antibody or antigen binding domain that specifically binds to EpCAM. “V_{L}CD3” and “V_{H}CD3” mean the light chain and heavy chain, respectively, of the variable region of anti-CD3 antibody or antigen binding domain that specifically binds to CD3.

In some embodiments, the first antigen binding domain comprises the V_{H}EpCAM and the V_{L}EpCAM. In some embodiments, the first antigen binding domain comprises: the V_{H}EpCAM comprising one, two or the three CDRs from SEQ ID NO:3, and/or the V_{L}EpCAM comprising one, two, or the three CDRs from SEQ ID NO:5. In some embodiments, the first antigen binding domain comprises: the V_{H}EpCAM comprising one, two or the three CDRs from SEQ ID NO:7, and/or the V_{L}EpCAM comprising one, two, or the three CDRs from SEQ ID NO:9. In some embodiments, the first antigen binding domain comprises: the V_{H}EpCAM comprising one, two or the three CDRs from SEQ ID NO:11, and/or the V_{L}EpCAM comprising one, two, or the three CDRs from SEQ ID NO:13. In some embodiments, the first antigen binding domain comprises: the V_{H}EpCAM comprising one, two or the three CDRs from SEQ ID NO:15, and/or the V_{L}EpCAM comprising one, two, or the three CDRs from SEQ ID NO:17. In some embodiments, the first antigen binding domain comprises: the V_{H}EpCAM comprising one, two or the three CDRs from SEQ ID NO:19, and/or the V_{L}EpCAM comprising one, two, or the three CDRs from SEQ ID NO:21. In some embodiments, the first antigen binding domain comprises: the V_{H}EpCAM comprising one, two or the three CDRs from SEQ ID NO:23, and/or the V_{L}EpCAM comprising one, two, or the three CDRs from SEQ ID NO:25.

In some embodiments, the V_{H}EpCAM and/or the V_{L}EpCAM are humanized. In some embodiments, the V_{H}CD3 and/or the V_{L}CD3 are humanized.

In some embodiments, the first antigen binding domain comprises the V_{H}EpCAM comprising the amino acid sequence of SEQ ID NO:27 and/or the V_{L}EpCAM comprising the amino acid sequence of SEQ ID NO:29. In some embodiments, the first antigen binding domain comprises the V_{H}EpCAM comprising the amino acid sequence of SEQ ID NO:31 and/or the V_{L}EpCAM comprising the amino acid of SEQ ID NO:33. In some embodiments, the first antigen binding domain comprises the V_{H}EpCAM comprising the amino acid sequence of SEQ ID NO:35 and/or the V_{L}EpCAM comprising the amino acid sequence of SEQ ID NO:37.

In some embodiments, the single-chain bispecific antibody further comprises a peptide linker between two variable regions (e.g., between V_{L}EpCAM and V_{H}EpCAM, between V_{H}EpCAM and V_{H}CD3, and/or between V_{H}CD3 and V_{L}CD3.)

In some embodiments, the bispecific antibody further comprises a human serum albumin sequence (HSA), for example, the bispecific antibody further comprises a HSA at the C-terminus of the bispecific antibody. The human serum albumin sequence may comprise the amino acid sequence of SEQ ID NO: 45 or SEQ ID NO:47. In some embodiments, the bispecific antibody further comprises a peptide linker between a variable region (e.g., V_{L}EpCAM, V_{H}EpCAM, V_{H}CD3, or V_{L}CD3) and the human serum albumin sequence.

The peptide linker provided herein (such as the peptide linker between two variable regions (e.g., between V_{L}EpCAM and V_{H}EpCAM, between V_{H}EpCAM and V_{H}CD3, or between V_{H}CD3 and V_{L}CD3) or the peptide linker between a variable region (e.g., V_{L}CD3) and the human serum albumin sequence) can comprise at least about any of 1 amino acid, 2 amino acids, 3 amino acids, 4 amino acids, 5 amino acids, 8 amino acids, 10 amino acids, 12 amino acids, 15 amino acids, 18 amino acids, 20 amino acids, 25 amino acids, or 30 amino acids. In some embodiments, the peptide linker comprises about any of 1 amino acid, 2 amino acids, 3 amino acids, 4 amino acids, 5 amino acids, 8 amino acids, 10 amino acids, 12 amino acids, 15 amino acids, 18 amino acids, 20 amino acids, 25 amino acids, or 30 amino acids. The peptide linker can be any one of the following: the amino acid sequence of SEQ ID NO:49; the amino acid sequence of SEQ ID NO:53; the amino acid sequence of SEQ ID NO:51; S; GS; GGS; GGGS (SEQ ID NO:63); GGGGSGGGGS (SEQ ID NO:64), GGGGSGGGGSGGGGSGGGGS (SEQ ID NO:65); GGGGSGGGGSGGGGSGGGGSGGGGS (SEQ ID NO:66); AAAGGSGG (SEQ ID NO:77); GGGGSGGRASGGGGS (SEQ ID NO:78); GGGGSGGRASGGGGSGGGGS (SEQ ID NO:67); STDGNT (SEQ ID NO:68); GGSGG (SEQ ID NO:69); SAKTTP (SEQ ID NO:70); SAKTTPKLGG (SEQ ID NO:71); RADAAP (SEQ ID NO:72); RADAAPTVS (SEQ ID NO:73); RADAAAAGGPGS (SEQ ID NO:74); RADAAAAGGGGSGGGGSGGGGSGGGGS (SEQ ID NO:75); and GGKGSGGKGTGGKGSGGKGS(SEQ ID NO:76).

In some embodiments, the peptide linker between V_{L}EpCAM and V_{H}EpCAM comprises amino acid sequence of SEQ ID NO:49. In some embodiments, the peptide linker between V_{H}CD3 and V_{L}CD3 comprises the amino acid sequence of SEQ ID NO:53. In some embodiments, the peptide linker between V_{H}EpCAM and V_{H}CD3 comprises the amino acid sequence of SEQ ID NO:51. In some embodiments, the peptide linker between the V_{L}CD3 and the human serum albumin sequence comprises the amino acid sequence of SEQ ID NO:51.

In some embodiments, the second antigen binding domain specifically binds to CD3ε, CD3γ, or CD3δ chain. In some embodiments, the second antigen binding domain comprises the V_{H}CD3 and/or V_{L}CD3. In some embodiments, the V_{H}CD3 comprises the amino acid sequence of SEQ ID NO:55. In some embodiments, the V_{L}CD3 comprises the amino acid sequence of SEQ ID NO:57. In some embodiments, the V_{H}CD3 comprises one, two, or the three CDRs from the amino acid sequence of SEQ ID NO:55. In some embodiments, the V_{L}CD3 comprises one, two, or the three CDRs from the amino acid sequence of SEQ ID NO:57. In some embodiments, the V_{H}CD3 is humanized. In some embodiments, the V_{L}CD3 is humanized.

A bispecific antibody described herein may comprise the amino acid sequence of SEQ ID NO:39, SEQ ID NO:41, or SEQ ID NO:43.

The polynucleotide construct used for production of a single chain bispecific antibody provided herein may further contain a signal peptide sequence (e.g., the signal peptide is added to N-terminus of a bispecific antibody provided herein). Any signal peptide sequence known to one skilled in the art may be used. The signal peptide sequences used in Example 8 may be used.

In some embodiments of a bispecific antibody provided herein, the bispecific antibody comprises a histidine tag (e.g., 6×His tag), for example, the bispecific antibody comprises a 6×His tag at the C-terminus of the antibody. In some embodiments of a bispecific antibody provided herein, the bispecific antibody does not comprise a histidine tag.

It is contemplated that the present invention encompasses not only the monoclonal antibodies described above, but also any fragments thereof containing the active binding region of the antibodies, such as Fab, F(ab′)₂, scFv, Fv fragments and the like. Such fragments can be produced from the monoclonal antibodies described herein using techniques well established in the art (Rousseaux et al. (1986), in Methods Enzymol., 121:663-69 Academic Press).

Methods of preparing antibody fragment are well known in the art. For example, an antibody fragment can be produced by enzymatic cleavage of antibodies with pepsin to provide a 100 Kd fragment denoted F(ab′)₂. This fragment can be further cleaved using a thiol reducing agent, and optionally a blocking group for the sulfhydryl groups resulting from cleavage of disulfide linkages, to produce 50 Kd Fab′ monovalent fragments. Alternatively, an enzymatic cleavage using papain produces two monovalent Fab fragments and an Fc fragment directly. These methods are described, for example, by U.S. Pat. Nos. 4,036,945 and 4,331,647 and references contained therein, which patents are incorporated herein by reference. Also, see Nisonoff et al. (1960), Arch Biochem. Biophys. 89: 230; Porter (1959), Biochem. J. 73: 119; Smyth (1967), Methods in Enzymology 11: 421-426.

Alternatively, the Fab can be produced by inserting DNA encoding Fab of the antibody into an expression vector for prokaryote or an expression vector for eukaryote, and introducing the vector into a prokaryote or eukaryote to express the Fab.

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

In some embodiments, the antibody of the invention may be modified using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

The antibody or polypeptide of the invention may be conjugated (for example, linked) to an agent, such as a therapeutic agent and a label. Examples of therapeutic agents are radioactive moieties, cytotoxins, or chemotherapeutic molecules.

The antibody (or polypeptide) of this invention may be linked to a label such as a fluorescent molecule, a radioactive molecule, an enzyme, or any other labels known in the art. As used herein, the term “label” refers to any molecule that can be detected. In a certain embodiment, an antibody may be labeled by incorporation of a radiolabeled amino acid. In a certain embodiment, biotin moieties that can be detected by marked avidin (e.g., streptavidin containing a fluorescent marker or enzymatic activity that can be detected by optical or colorimetric methods) may be attached to the antibody. In certain embodiments, a label may be incorporated into or attached to another reagent which in turn binds to the antibody of interest. For example, a label may be incorporated into or attached to an antibody that in turn specifically binds the antibody of interest. In certain embodiments, the label or marker can also be therapeutic. Various methods of labeling polypeptides and glycoproteins are known in the art and may be used. Certain general classes of labels include, but are not limited to, enzymatic, fluorescent, chemiluminescent, and radioactive labels. Examples of labels for polypeptides include, but are not limited to, the following: radioisotopes or radionucleoides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., fluorescein isothocyanate (FITC), rhodamine, lanthanide phosphors, phycoerythrin (PE)), enzymatic labels (e.g., horseradish peroxidase, β-galactosidase, luciferase, alkaline phosphatase, glucose oxidase, glucose-6-phosphate dehydrogenase, alcohol dehyrogenase, malate dehyrogenase, penicillinase, luciferase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The invention also provides pharmaceutical compositions comprising antibodies or polypeptides described herein, and a pharmaceutically acceptable carrier or excipients. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

In some embodiments, the invention provides compositions (described herein) for use in any of the methods described herein, whether in the context of use as a medicament and/or use for manufacture of a medicament.

### Polynucleotides, Vectors and Host Cells

The invention also provides polynucleotides comprising a nucleotide sequence encoding any of the antibodies and polypeptides described herein. In some embodiments, the polypeptides comprise the sequences of light chain and/or heavy chain variable regions. In some embodiments, the polynucleotides comprise one or more nucleic acid sequences encoding an anti-EpCAM antibody described herein. In some embodiments, the polynucleotides comprise one or more nucleic acid sequences encoding a single chain bispecific antibody described herein.

In some embodiments, the polynucleotides comprise a nucleic acid sequence encoding a heavy chain variable region comprising one, two or the three CDRs of SEQ ID NO:3 and a nucleic acid sequence encoding a light chain variable region comprising one, two, or the three CDRs of SEQ ID NO:5. In some embodiments, the polynucleotides comprise a nucleic acid sequence encoding a heavy chain variable region comprising one, two or the three CDRs of SEQ ID NO:7 and a nucleic acid sequence encoding a light chain variable region comprising one, two, or the three CDRs of SEQ ID NO:9. In some embodiments, the polynucleotides comprise a nucleic acid sequence encoding a heavy chain variable region comprising one, two or the three CDRs of SEQ ID NO:11 and a nucleic acid sequence encoding a light chain variable region comprising one, two, or the three CDRs of SEQ ID NO:13. In some embodiments, the polynucleotides comprise a nucleic acid sequence encoding a heavy chain variable region comprising one, two or the three CDRs of SEQ ID NO:15 and a nucleic acid sequence encoding a light chain variable region comprising one, two, or the three CDRs of SEQ ID NO:17. In some embodiments, the polynucleotides comprise a nucleic acid sequence encoding a heavy chain variable region comprising one, two or the three CDRs of SEQ ID NO:19 and a nucleic acid sequence encoding a light chain variable region comprising one, two, or the three CDRs of SEQ ID NO:21. In some embodiments, the polynucleotides comprise a nucleic acid sequence encoding a heavy chain variable region comprising one, two or the three CDRs of SEQ ID NO:23 and a nucleic acid sequence encoding a light chain variable region comprising one, two, or the three CDRs of SEQ ID NO:25.

In some embodiments, the polynucleotides comprise a nucleic acid sequence encoding a heavy chain variable region comprising the amino acid sequence of SEQ ID NO:3 and/or a nucleic acid sequence encoding a light chain variable region comprising the amino acid sequence of SEQ ID NO:5. In some embodiments, the polynucleotides comprise a nucleic acid sequence encoding a heavy chain variable region comprising the amino acid sequence of SEQ ID NO:7 and/or a nucleic acid sequence encoding a light chain variable region comprising the amino acid sequence of SEQ ID NO:9. In some embodiments, the polynucleotides comprise a nucleic acid sequence encoding a heavy chain variable region comprising the amino acid sequence of SEQ ID NO:11 and/or a nucleic acid sequence encoding a light chain variable region comprising the amino acid sequence of SEQ ID NO:13. In some embodiments, the polynucleotides comprise a nucleic acid sequence encoding a heavy chain variable region comprising the amino acid sequence of SEQ ID NO:15 and/or a nucleic acid sequence encoding a light chain variable region comprising the amino acid sequence of SEQ ID NO:17. In some embodiments, the polynucleotides comprise a nucleic acid sequence encoding a heavy chain variable region comprising the amino acid sequence of SEQ ID NO:19 and/or a nucleic acid sequence encoding a light chain variable region comprising the amino acid sequence of SEQ ID NO:21. In some embodiments, the polynucleotides comprise a nucleic acid sequence encoding a heavy chain variable region comprising the amino acid sequence of SEQ ID NO:23 and/or a nucleic acid sequence encoding a light chain variable region comprising the amino acid sequence of SEQ ID NO:25.

In some embodiments, the polynucleotides comprise one or more nucleic acid sequences encoding a single-chain bispecific antibody comprising (a) a first antigen binding domain that specifically binds to human EpCAM (e.g., specifically binds to an epitope within amino acids 24-63 of human EpCAM), wherein the first antigen binding domain comprises a heavy chain variable region (V_{H}EpCAM) and/or a light chain variable region (V_{L}EpCAM); and/or (b) a second antigen binding domain that specifically binds to human CD3 antigen, wherein the second antigen binding domain comprises a heavy chain variable region (V_{H}CD3) and/or a light chain variable region (V_{L}CD3); wherein the variable regions are arranged in an order (e.g., arranged from N-terminus to C-terminus in the order V_{L}EpCAM-V_{H}EpCAM-V_{H}CD3-V_{L}CD3).

In some embodiments, the polynucleotides comprise the nucleic acid sequence of SEQ ID NO:4 and/or the nucleic acid sequence of SEQ ID NO:6. In some embodiments, the polynucleotides comprise the nucleic acid sequence of SEQ ID NO:8 and/or the nucleic acid sequence of SEQ ID NO:10. In some embodiments, the polynucleotides comprise the nucleic acid sequence of SEQ ID NO:12 and/or the nucleic acid sequence of SEQ ID NO:14. In some embodiments, the polynucleotides comprise the nucleic acid sequence of SEQ ID NO:16 and/or the nucleic acid sequence of SEQ ID NO:18. In some embodiments, the polynucleotides comprise the nucleic acid sequence of SEQ ID NO:20 and/or the nucleic acid sequence of SEQ ID NO:22. In some embodiments, the polynucleotides comprise the nucleic acid sequence of SEQ ID NO:24 and/or the nucleic acid sequence of SEQ ID NO:26.

In some embodiments, the polynucleotides comprise the nucleic acid sequence of SEQ ID NO:28 and/or the nucleic acid sequence of SEQ ID NO:30. In some embodiments, the polynucleotides comprise the nucleic acid sequence of SEQ ID NO:32 and/or the nucleic acid sequence of SEQ ID NO:34. In some embodiments, the polynucleotides comprises the nucleic acid sequence of SEQ ID NO:36 and/or the nucleic acid sequence of SEQ ID NO:38. In some embodiments, the polynucleotides comprise the nucleic acid sequence of SEQ ID NO:56 and/or the nucleic acid sequence of SEQ ID NO:58. In some embodiments, the polynucleotides comprise the nucleic acid sequence of SEQ ID NO:40. In some embodiments, the polynucleotides comprise the nucleic acid sequence of SEQ ID NO:42. In some embodiments, the polynucleotides comprise the nucleic acid sequence of SEQ ID NO:44.

It is appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Thus, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein can, but need not, have an altered structure or function. Alleles can be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides can be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

The invention also provides vectors (e.g., cloning vectors, expression vectors) comprising a nucleic acid sequence encoding any of the polypeptides (including antibodies) described herein. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a polynucleotide comprising one or more amino acid sequences encoding an antibody described herein (e.g., an anti-EpCAM antibody). In some embodiments, the vector contains a polynucleotide comprising one or more amino acid sequences encoding a single-chain bispecific antibody described herein.

The invention also provides host cells comprising any of the polynucleotides described herein (e.g., polynucleotides comprising one or more amino acid sequences encoding an antibody described herein such as an anti-EpCAM antibody) or vectors described herein (e.g., a vector containing polynucleotides comprising one or more amino acid sequences encoding an antibody described herein such as an anti-EpCAM antibody). Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis). In some embodiments, the host cell comprises a polynucleotide comprising one or more amino acid sequences encoding a single-chain bispecific antibody described herein.

### Diagnostic Uses

The present invention provides a method of using the antibodies (e.g., an anti-EpCAM antibody), polypeptides and polynucleotides (e.g., polynucleotides comprising one or more amino acid sequences encoding an anti-EpCAM antibody) of the present invention for detection, diagnosis and monitoring of a disease, disorder or condition associated with the EpCAM expression (either increased or decreased relative to a normal sample, and/or inappropriate expression, such as presence of expression in tissues(s) and/or cell(s) that normally lack the EpCAM expression). In some embodiments, a single-chain bispecific antibody described herein or polynucleotide comprising one or more amino acid sequences encoding a single-chain bispecific antibody described herein is used.

In some embodiments, the method comprises detecting the EpCAM expression in a sample obtained from a subject suspected of having cancer, such as breast, colorectal, gastric, lung, prostate, pancreatic, pharynx, and ovarian cancer. Preferably, the method of detection comprises contacting the sample with an antibody, polypeptide, or polynucleotide of the present invention and determining whether the level of binding differs from that of a control or comparison sample. The method is also useful to determine whether the antibodies or polypeptides described herein are an appropriate treatment for the patient.

As used herein, the term “a sample” or “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). “A sample” or “a biological sample” further refers to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. Most often, the sample has been removed from an animal, but the term “a sample” or “a biological sample” can also refer to cells or tissue analyzed in vivo, i.e., without removal from animal. Typically, “a sample” or “a biological sample” will contain cells from the animal, but the term can also refer to non-cellular biological material, such as non-cellular fractions of blood, saliva, or urine, that can be used to measure the cancer-associated polynucleotide or polypeptides levels. “A sample” or “a biological sample” further refers to a medium, such as a nutrient broth or gel in which an organism has been propagated, which contains cellular components, such as proteins or nucleic acid molecules.

In one embodiment, the cells or cell/tissue lysate are contacted with an antibody and the binding between the antibody and the cell is determined. When the test cells are shown binding activity as compared to a control cell of the same tissue type, it may indicate that the test cell is cancerous. In some embodiments, the test cells are from human tissues.

Various methods known in the art for detecting specific antibody-antigen binding can be used. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-galactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art.

In some embodiments, the polypeptides including antibodies of the invention need not be labeled, and the presence thereof can be detected using a labeled antibody which binds to the antibodies of the invention.

The antibodies of the present invention can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, or ³H) so that the cells or tissue of interest can be localized using immunoscintiography.

The antibody may also be used as staining reagent in pathology using techniques well known in the art.

### Therapeutic Uses

The antibodies of the present invention are capable of inducing cancer cell apoptosis in vitro in the absence of cytotoxin conjugate, immune effector functions, or cross-linking agents. These antibodies may have more potent anti-cancer effect in vivo. Thus, the present invention provides therapeutic uses of the antibodies (e.g., an anti-EpCAM antibody described herein) and polypeptides of the present invention in treating a cancer, delaying development of a cancer, and/or preventing a cancer, such as breast, colorectal, gastric, lung, prostate, pancreatic, pharynx, and ovarian cancer. Any cancer may be treated, as long as the cancer cell expresses the EpCAM recognized by the antibodies described herein. The method may further comprise a step of detecting the binding between an antibody or a polypeptide described herein and a tumor or cancer cell in an individual to be treated.

In some embodiments, the antibody is a single-chain bispecific antibody provided herein. In some embodiments, the single-chain bispecific antibody comprises (a) a first antigen binding domain that specifically binds to human EpCAM (e.g., specifically binds to an epitope within amino acids 24-63 of human EpCAM), wherein the first antigen binding domain comprises a heavy chain variable region (V_{H}EpCAM) and/or a light chain variable region (V_{L}EpCAM); and/or (b) a second antigen binding domain that specifically binds to human CD3 antigen, wherein the second antigen binding domain comprises a heavy chain variable region (V_{H}CD3) and/or a light chain variable region (V_{L}CD3); wherein the variable regions are arranged in an order (e.g., arranged from N-terminus to C-terminus in the order V_{L}EpCAM-V_{H}EpCAM-V_{H}CD3-V_{L}CD3).

Generally, an effective amount of a composition comprising an antibody or a polypeptide is administered to a subject in need of treatment, thereby inhibiting growth of the cancer cell and/or inducing death of the cancer cell. Preferably the composition is formulated with a pharmaceutically acceptable carrier.

In one embodiment, the composition is formulated for administration by intraperitoneal, intravenous, subcutaneous, and intramuscular injections, and other forms of administration such as oral, mucosal, via inhalation, sublingually, etc.

In another embodiment, the present invention also contemplates administration of a composition comprising the antibodies or polypeptides of the present invention conjugated to other molecules, such as detectable labels, or therapeutic or cytotoxic agents. The agents may include, but are not limited to radioisotopes, toxins, toxoids, inflammatory agents, enzymes, antisense molecules, peptides, cytokines, or chemotherapeutic agents. Methods of conjugating the antibodies with such molecules are generally known to those of skilled in the art. See, e.g., PCT publications WO 92/08495; WO 91/14438; WO 89/12624; U.S. Pat. No. 5,314,995; and EP 396,387; the disclosures of which are incorporated herein by reference in their entireties.

In one embodiment, the composition comprises an antibody or polypeptide conjugated to a cytotoxic agent. Cytotoxic agents can include any agents that are detrimental to cells. A preferred class of cytotoxic agents that can be conjugated to the antibodies or fragments may include, but are not limited to paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The dosage required for the treatment depends on the choice of the route of administration, the nature of the formulation, the nature of the subject's illness, the subject's size, weight, surface area, age and sex; other drugs being administered, and the judgment of the attending physician. Suitable dosages are in the range of 0.01-1000.0 mg/kg.

Generally, any of the following doses may be used: a dose of at least about 50 mg/kg body weight; at least about 10 mg/kg body weight; at least about 3 mg/kg body weight; at least about 1 mg/kg body weight; at least about 750 μg/kg body weight; at least about 500 μg/kg body weight; at least about 250 μg/kg body weight; at least about 100 μg/kg body weight; at least about 50 μg/kg body weight; at least about 10 μg/kg body weight; at least about 1 μg/kg body weight, or less, is administered. For repeated administrations over several days or longer, depending on the condition, the treatment is sustained until a desired suppression of disease symptoms occurs. An exemplary dosing regimen comprises administering a weekly dose of about 6 mg/kg of the antibody. However, other dosage regimens may be useful, depending on the pattern of pharmacokinetic decay that the practitioner wishes to achieve. Empirical considerations, such as the half-life, generally will contribute to determination of the dosage. The progress of this therapy is easily monitored by conventional techniques and assays.

In some subjects, more than one dose may be required. Frequency of administration may be determined and adjusted over the course of therapy. For example, frequency of administration may be determined or adjusted based on the type and stage of the cancer to be treated, whether the agent is administered for preventive or therapeutic purposes, previous therapy, the patient's clinical history and response to the agent, and the discretion of the attending physician. Typically the clinician will administer a therapeutic antibody (such as a chimeric 5F1 antibody), until a proper dosage is reached to achieves the desired result. In some cases, sustained continuous release formulations of antibodies may be appropriate. Various formulations and devices for achieving sustained release are known in the art.

In one embodiment, dosages for the antibodies or polypeptides may be determined empirically in subjects who have been given one or more administration(s). Subjects are given incremental dosages of the antibodies or polypeptides. To assess efficacy of the antibodies or polypeptides, markers of the disease symptoms such as EpCAM can be monitored. Efficacy in vivo can also be measured by assessing tumor burden or volume, the time to disease progression (TDP), and/or determining the response rates (RR).

Administration of an antibody or polypeptide in accordance with the method in the present invention can be continuous or intermittent, depending, for example, upon the recipient's physiological condition, whether the purpose of the administration is therapeutic or prophylactic, and other factors known to skilled practitioners. The administration of an antibody or a polypeptide may be essentially continuous over a preselected period of time or may be in a series of spaced dose.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In another embodiment, the composition can comprise one or more anti-cancer agents, one or more antibodies described herein, or with an antibody or polypeptide that binds to a different antigen. Such composition can contain at least one, at least two, at least three, at least four, at least five different antibodies. The antibodies and other anti-cancer agents may be in the same formulation (e.g., in a mixture, as they are often denoted in the art), or in separate formulations but are administered concurrently or sequentially, are particularly useful in treating a broader range of population of individuals.

A polynucleotide encoding any of the antibodies or polypeptides of the present invention can also be used for delivery and expression of any of the antibodies or polypeptides of the present invention in a desired cell. It is apparent that an expression vector can be used to direct expression of the antibody or polypeptide. The expression vector can be administered by any means known in the art, such as intraperitoneally, intravenously, intramuscularly, subcutaneously, intrathecally, intraventricularly, orally, enterally, parenterally, intranasally, dermally, sublingually, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding any of the antibodies or polypeptides of the present invention can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al. (1990), Proc. Natl. Acad. Sci. USA, 87:3655; Wu et al. (1991), J. Biol. Chem. 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol.

The therapeutic polynucleotides and polypeptides of the present invention can be delivered using gene delivery vehicles. The gene delivery vehicle can be of viral or non-viral origin (see generally, Jolly (1994), Cancer Gene Therapy 1:51; Kimura (1994), Human Gene Therapy 5:845; Connelly (1985), Human Gene Therapy 1:185; and Kaplitt (1994), Nature Genetics 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP Patent No. 0 345 242; alphavirus-based vectors, e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors, e.g., PCT Publication Nos. WO 94/12649, WO 93/03769; WO 93/19191; WO 94/28938; WO 95/11984 and WO 95/00655. Administration of DNA linked to killed adenovirus as described in Curiel (1992), Hum. Gene Ther. 3:147 can also be employed.

Non-viral delivery vehicles and methods can also be employed, including, but are not limited to, polycationic condensed DNA linked or unlinked to killed adenovirus alone (see, e.g., Curiel (1992), Hum. Gene Ther. 3:147); ligand-linked DNA (see, e.g., Wu (1989), J. Biol. Chem. 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes.

Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent NO. 0 524 968. Additional approaches are described in Philip (1994), Mol. Cell Biol. 14:2411 and in Woffendin (1994), PNAS 91:1581.

Additionally, the invention provides a method of treating a cancer, delaying development of a cancer, and/or preventing a cancer in an individual comprising a) administering to the individual an effective amount of a composition comprising an antibody of the present invention and b) applying a second cancer therapy to the individual. In some embodiments, the second therapy includes surgery, radiation, hormone therapy, gene therapy, other antibody therapy, and chemotherapy. The composition comprising the antibody and the second therapy can be applied concurrently (e.g., simultaneous administration) and/or sequentially (e.g., sequential administration). For example, the composition comprising the antibody and the second therapy are applied with a time separation of no more than about 15 minutes, such as no more than about any of 10, 5, or 1 minutes. Alternatively, the composition comprising the antibody and the second therapy are applied with a time separation of more than about 15 minutes, such as about any of 20, 30, 40, 50, or 60 minutes, 1 day, 2 days, 3 days, 1 week, 2 weeks, or 1 month, or longer.

The composition comprising an antibody of the present invention can be administered sequentially or concurrently with one or more other therapeutic agents such as chemotherapeutic agents (such as 5-FU, 5-FU/MTX, 5-FU/Leucovorin, Levamisole, Irinotecan, Oxaliplatin, Capecitabin, or Uracil/Tegafur), immunoadjuvants, growth inhibitory agents, cytotoxic agents and cytokines, etc. The amounts of the antibody and the therapeutic agent depend on what type of drugs are used, the pathological condition being treated, and the scheduling and routes of administration but would generally be less than if each were used individually.

Following administration of the composition comprising the antibody described herein, the efficacy of the composition can be evaluated both in vitro and in vivo by various methods well known to one of ordinary skill in the art. Various animal models are well known for testing anti-cancer activity of a candidate composition. These include human tumor xenografting into athymic nude mice or scid/scid mice, or genetic murine tumor models such as p53 knockout mice. The in vivo nature of these animal models make them particularly predictive of responses in human patients. Such models can be generated by introducing cells into syngeneic mice using standard techniques, e.g., subcutaneous injection, tail vein injection, spleen implantation, intraperitoneal implantation and implantation under the renal capsule, etc.

### Articles of Manufacture and Kits

The invention also provides articles of manufacture or kits for use in the instant methods. Articles of manufacture or kits of the invention include one or more containers comprising a purified antibody or a polypeptide described herein and instructions for use in accordance with any of the methods of the invention described herein. In some embodiments, these instructions comprise a description of administration of the antibody to treat, delay development of, and/or preventing a cancer, such as breast, colorectal, gastric, lung, prostate, pancreatic, pharynx, and ovarian cancer, according to any of the methods described herein. The kit may further comprise a description of selecting an individual suitable for treatment based on identifying whether that individual has the disease and the stage of the disease, or whether EpCAM is expressed on the cancer cells in the individual.

In some embodiments, the articles of manufacture or kits for detecting a cancer cell in a sample comprise an antibody or a polypeptide described herein and reagents for detecting binding of the antibody or the polypeptide to a cell in the sample.

The instructions relating to the use of the antibodies or polypeptides to treat, delay development of, and/or prevent a cancer generally include information as to dosage, dosing schedule, and route of administration for the intended treatment. The containers may be unit doses, bulk packages (e.g., multi-dose packages) or sub-unit doses. Instructions supplied in the articles of manufacture or kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying development of, and/or preventing a cancer described herein. Instructions may be provided for practicing any of the methods described herein.

The articles of manufacture or kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. An article of manufacture or a kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-EpCAM antibody described herein. The container may further comprise a second pharmaceutically active agent.

The articles of manufacture or kits may optionally provide additional components such as buffers and interpretive information. Normally, the kit comprises a container and a label or package insert(s) on or associated with the container.

The articles of manufacture or kits may include any one of an anti-EpCAM antibody described herein. In some embodiments, the article of manufacture or kit includes a single-chain bispecific antibody provided herein. In some embodiments, the single-chain bispecific antibody comprises (a) a first antigen binding domain that specifically binds to human EpCAM (e.g., specifically binds to an epitope within amino acids 24-63 of human EpCAM), wherein the first antigen binding domain comprises a heavy chain variable region (V_{H}EpCAM) and/or a light chain variable region (V_{L}EpCAM); and/or (b) a second antigen binding domain that specifically binds to human CD3 antigen, wherein the second antigen binding domain comprises a heavy chain variable region (V_{H}CD3) and/or a light chain variable region (V_{L}CD3); wherein the variable regions are arranged in an order (e.g., arranged from N-terminus to C-terminus in the order V_{L}EpCAM-V_{H}EpCAM-V_{H}CD3-V_{L}CD3).

### EXAMPLES

The following Examples are provided to illustrate but not to limit the invention.

### Example 1

### Generation of Anti-hEpCAM Antibodies

(1) Generation of Anti-hEpCAM Antibodies with Cancer Cells Immunization

Human breast carcinoma cell line, T-47D (BCRC 60250) and choriocarcinoma cell line, BeWo (CCRC 60073) were purchased from Food Industry Research and Development Institute, Hsin-chu, Taiwan. T-47D was maintained in RPMI 1640 medium (GIBCO BRL) with 2 mM L-glutamine adjusted to contain 1.5 g/L sodium bicarbonate, 4.5 g/L glucose, 10 mM HEPES, 1.0 mM sodium pyruvatem, and 0.2 Units/ml bovine insulin, 90%; and supplemented with 10% fetal bovine serum (FBS), 100 units/ml of penicillin and 100 μg/ml of streptomycin (GIBCO BRL) at 37° C. in a humidified atmosphere of 5% CO₂. BeWo was grown in 85% Ham's F12K medium (GIBCO BRL) with 2 mM L-glutamine adjusted to contain 1.5 g/L sodium bicarbonate, 85%; and supplemented with 15% FBS, 100 units/ml of penicillin and 100 μg/ml of streptomycin (GIBCO BRL) at 37° C. in a humidified atmosphere of 5% CO₂.

Balb/c mice were immunized three times with 1×10⁷ T-47D or BeWo cells in 500 μl PBS every two weeks. Final boost with the same amount of cells was given 3 to 5 days before the fusion where the spleen cells were harvested and fused with X63 myeloma cells. Hybridomas were grown and selected with DMEM supplemented with 10% FBS (Hyclone) and HAT (Hybri-Max®, Sigma H0262, at a final concentration of 100 μM hypoxanthine, 0.4 μM aminopterin, 16 μM thymidine). Three hybridoma cell lines m290-1G10, m342-12H8.3, and m342-15F11.1 which secrete the monoclonal antibody 1G10 (IgG₁, K), 12H8 (IgG₂ₐ, K), and 15F11 (IgG2a, K), respectively have been generated.

(2) Generation of Anti-hEpCAM Antibodies with Recombinant Protein Immunization

The cDNA encoding human EpCAM extracellular domain (aa 1-264) was amplified by PCR. For facilitating the purification of expressed recombinant protein, human EpCAM extracellular domain were expressed as fusion proteins with constant region of human immunoglobulin gamma 1 heavy chain (EpCAM Exd-Cr1). The expression plasmids (pcDNAS/FRT-EpEXD) were stably transfected into Chinese hamster ovary (CHO) (Invitrogen R758-07) cells by Lipofectamine 2000 (Invitrogen, Cat. No. 11668-500) according to the manufacturer's instruction. The stable cell line CHO/Exd-Cr1 was grown in Ham's F12, containing 10% FBS and 600 ug/ml Hygromycin B (C.A. IN-10687-010), Ultra low-Ig (Invitrogen, Cat. No. 16250-078). The culture medium was collected and the hEpCAM extracellular domain (EpCAM Exd-Cr1) recombinant protein was purification using protein A Sepharose (Amersham Bioscience).

Naïve Balb/c mice were primed with 50 micro-gram of purified EpCAM Exd-Cr1 recombinant protein in CFA and following every two weeks boosted with 10 micro-gram of purified recombinant protein in IFA for three times. Three days after the final boost, the mouse spleen cells were harvested and fused with X63 myeloma cells. Hybridomas were selected with DMEM supplemented with 10% FBS (Hyclone) and HAT (Hybri-Max®, Sigma H0262, at a final concentration of 100 μM hypoxanthine, 0.4 μM aminopterin, 16 μM thymidine). The hybridoma clones m322-2D11.19, m322-8B8.16, m338-1F10.3, m338-6D11.8, m338-4D2.20 and m338-6B2.3 which secrete the monoclonal antibody 2D11 (IgG₁, K), 8B8 (IgG₁, K), 1F10 (IgG₁, λ1), 6D11 (IgG₁, K), 4D2 (IgG₁, K) and 6B2 (IgG₁, K) respectively have been identified and were further characterized.

### Example 2

### Characterization of Anti-EpCAM Clones Generated

### (1) Establishment of CHO/EpCAM Cell Line

The cDNAs encoding full-length of human EpCAM (aa 1-314) were amplified by PCR from the cDNA pool generated from T-47D cells and cloned into the modified pcDNA 3.1/Myc-His(+) A vector (Invitrogen), followed by transfecting into Chinese hamster ovary (CHO) cells at 80-90% confluence in 6-well culture dishes, using Lipofectamine 2000 (Invitrogen, Cat. No. 11668-500). Transfectants were selected in F12/10% FBS medium containing 600 ug/ml Hygromycin B (C.A. IN-10687-010). Transfected cells expressing hEpCAM were identified by flow cytometry with anti-EpCAM antibodies.

### (2) Binding of Selected Anti-EpCAM Clones to CHO/EpCAM Cells

1×10⁵ CHO/EpCAM or CHO parental cells were seeded in each well of a v-bottomed 96-well plate and incubated with purified anti-EpCAM antibodies or mouse IgG control antibody 9E10 at concentration of 0.1 μg/ml. A 300× dilution of human cell hyper-immune serum (HPS 300×) was used as binding positive control for both cell lines here. After 1 hr incubation at 4° C., cells were washed twice with 200 μl FACS buffer (1×PBS containing 1% FBS), stained with 100 μl of 1 μg/ml goat-anti-mouse IgG-PE (Southern Biotech, Cat. No. 1032-09) in FACS buffer and then incubated at 4° C. for 30 min. Cells were washed thrice with FACS buffer and analyzed by flow cytometry (BD LSR, BD Life Sciences).

TABLE 2
Binding (FACS, Mean fluorescence intensity (MFI)) of anti-EpCAM
antibodies for CHO/EpCAM and CHO cells at 0.1 μg/ml.

| Clone | CHO/EpCAM | CHO |
|---|---|---|
| 12H8 | 6649 | 5 |
| 15F11 | 64 | 5 |
| 1G10 | 6178 | 5 |
| 2D11 | 3718 | 6 |
| 6D11 | 1347 | 5 |
| 4D2 | 1745 | 5 |
| 1F10 | 5634 | 6 |
| 6B2 | 1348 | 5 |
| 8B8 | 1661 | 5 |
| 9E10 | 6 | 5 |
| HPS 300x | 95 | 61 |

The results in Table 2 show that all the clones are human EpCAM specific, as they only bind to CHO/EpCAM but not parental CHO cells. Furthermore, the clones displayed different binding ability toward CHO/EpCAM cells, ranging from MFI of 64 (clone 15F11) to 6649 (clone 12H8).

### Example 3

### Cytotoxic Effect of Anti-EpCAM Antibodies in Cancer Cell Lines

### (1) Cytotoxicity of Anti-EpCAM Antibodies in Variant Cancer Cell Lines.

For antibody cytotoxicity assay, 4-5×10⁴ cancer cells were seeded to each well of a flat-bottomed 96-well plate, and then different concentrations (1, 3, or 10 μg/ml) of anti-EpCAM (12H8, 1G10, 1F10, 2D11, 6D11 and 4D2) or control (9E10) antibody, diluted in the medium was added into the cells. After a 17-20 hr incubation at 37° C., cells were stained with 0.3 μl of FITC-conjugated Annexin V (Strong Biotech Corp. Cat. No. AVK250) in 50 μl Annexin V binding buffer (Strong Biotech Corp. Cat. No. AVK250) at RT for 20 min. After wash, the cells were then stained with 0.3 μl of propidium iodide in 200 μl Annexin V binding buffer. Annexin V and Propidium Iodide were used as a joint indicator for cell death. The data were acquired with BD FACSCalibur based on an acquisition of 3,000-5,000 cells and analyzed with the Cell Quest software. The combined percentages of the Annexin V+/PI+, Annexin V+/PI− and Annexin V−/PI+populations were considered as dead cells.

TABLE 3
Cytotoxic effect of anti-EpCAM antibodies
in EpCAM-positive cancer cells.

| | | | | | Untreated |
|---|---|---|---|---|---|
| Cancer type& cell | mAb | Concentration tested μg/ml | | | medium |
| line tested | tested | 1.0 | 3.0 | 10 | control |
| Breast cancer T- | 12H8 | 49 | 54 | 56 | 20 |
| 47D | 1G10 | 45 | 58 | 64 | |
| | 1F10 | 51 | 50 | 50 | |
| | 2D11 | 36 | 48 | 45 | |
| | 6D11 | 25 | 40 | 44 | |
| | 4D2 | 35 | 44 | 45 | |
| | 9E10 | 14 | 16 | 18 | |
| Colorectal cancer | 12H8 | 61 | 67 | 72 | 15 |
| DLD-1 | 1G10 | 28 | 40 | 60 | |
| | 1F10 | 65 | 65 | 61 | |
| | 2D11 | 18 | 41 | 48 | |
| | 6D11 | 28 | 53 | 62 | |
| | 4D2 | 31 | 51 | 62 | |
| | 9E10 | 14 | 14 | 15 | |
| Gastric cancer | 12H8 | 67 | 62 | 66 | 35 |
| NCI-N87 | 1G10 | 50 | 60 | 60 | |
| | 1F10 | 57 | 57 | 57 | |
| | 2D11 | 45 | 48 | 51 | |
| | 6D11 | 41 | 51 | 57 | |
| | 4D2 | 49 | 52 | 55 | |
| | 9E10 | 35 | 33 | 33 | |
| Lung cancer NCI- | 12H8 | 24 | 29 | 34 | 19 |
| H520 | 1G10 | 32 | 41 | 55 | |
| | 1F10 | 40 | 46 | 57 | |
| | 2D11 | 41 | 44 | 52 | |
| | 6D11 | 38 | 42 | 49 | |
| | 4D2 | 24 | 30 | 32 | |
| | 9E10 | 29 | 24 | 25 | |
| Prostate cancer | 12H8 | 61 | 67 | 58 | 20 |
| LNCaP | 1G10 | 42 | 55 | 45 | |
| | 1F10 | 47 | 53 | 48 | |
| | 2D11 | 40 | 39 | 40 | |
| | 6D11 | 38 | 43 | 44 | |
| | 4D2 | 49 | 52 | 50 | |
| | 9E10 | 26 | 28 | 28 | |
| Pharynx cancer | 12H8 | 42 | 43 | 44 | 18 |
| FaDu | 1G10 | 40 | 50 | 51 | |
| | 1F10 | 44 | 42 | 45 | |
| | 2D11 | 34 | 45 | 46 | |
| | 6D11 | 26 | 39 | 37 | |
| | 4D2 | 37 | 43 | 46 | |
| | 9E10 | 17 | 15 | 14 | |
| Ovarian cancer | 12H8 | 59 | 62 | 63 | 25 |
| NIH:OVCAR-3 | 1G10 | 40 | 50 | 52 | |
| | 1F10 | 53 | 57 | 59 | |
| | 2D11 | 43 | 55 | 56 | |
| | 6D11 | 46 | 52 | 55 | |
| | 4D2 | 40 | 51 | 56 | |
| | 9E10 | 22 | 25 | 27 | |

Table 3 summarizes the result of Annexin V and PI staining (indicating cytotoxic effect) of various cancer cells after overnight incubation with anti-EpCAM mAbs (12H8, 1G10, 1F10, 2D11, 6D11 and 4D2) or 9E10 (isotype control) at 1, 3 and 10 μg/ml. The result shows that clone 12H8, 1G10, 1F10, 2D11, 6D11 and 4D2 can induce substantial cell death in breast (T-47D), colorectal (DLD-1), gastric (NCI-N87), lung (NCI-H520), prostate (LNCaP), pharynx (FaDu) and ovarian (NIH:OVCAR-3) cancer cells.

### (2) Not All Anti-EpCAM Antibodies Induce Substantial Cytotoxicity

5×10⁴ NCI-H358 cells were seeded to each well of a 96-well plate, and then 10 μg/ml of anti-EpCAM or control (9E10) mAbs diluted in the medium was added into the cells. After a 17-hr incubation at 37° C., the cells were stained with 0.3 μl of FITC-conjugated Annexin V (Strong Biotech Corp. Cat. No. AVK250) in 50 μl Annexin V binding buffer (Strong Biotech Corp. Cat. No. AVK250) at RT for 20 min. After wash, the cells were then stained with 0.3 μl of propidium iodide in 200 μl Annexin V binding buffer. Annexin V and Propidium Iodide were used as a joint indicator for measuring cell death. The data were acquired with BD FACSCalibur based on an acquisition of 3,000-5,000 cells and analyzed with the Cell Quest software. The combined percentages of the Annexin V+/PI+, Annexin V+/PI− and Annexin V−/PI+populations were considered as dead cells.

TABLE 4
Cell death in NCI-H358 lung cancer cells induced by anti-EpCAM mAbs

| mAb | 9E10 | 12H8 | 1F10 | 1G10 | 2D11 | 6D11 | 4D2 | 6B2 | 15F11 | 8B8 |
|---|---|---|---|---|---|---|---|---|---|---|
| Exp1 | 1 | 36 | 32 | 46 | 41 | 45 | 43 | 7 | 4 | 2 |
| Exp2 | −1 | 33 | 30 | 42 | 32 | 40 | 41 | 2 | 2 | 31 |
| Exp3 | −4 | 31 | 40 | 48 | 47 | 44 | 45 | −4 | −5 | 5 |
| Exp4 | 4 | 40 | 39 | 47 | 44 | 43 | 46 | 15 | 4 | 12 |
| Exp5 | −1 | 28 | 37 | 42 | 33 | 40 | 43 | 1 | −5 | 18 |
| Mean ± | −0.2 ± 1.3 | 33.6 ± 2.1 | 35.6 ± 2.0 | 45.0 ± 1.3 | 39.4 ± 3.0 | 42.4 ± 1.0 | 43.6 ± 0.9 | 4.2 ± 3.2 | 0.0 ± 2.1 | 13.6 ± 5.2 |
| SEM | | | | | | | | | | |
| P | | P < | P < | P < | P < | P < | P < | 0.24 | 0.94 | 0.03 |
| value | | 0.01 | 0.01 | 0.01 | 0.01 | 0.01 | 0.01 | | | |

% of Annexin V + PI staining (untreated background subtracted)
*t-test P value < 0.01 (compared to treatment with isotype control antibody 9E10)

Table 4 summarizes the cell death induced by anti-EpCAM clones in NCI-H358 lung cancer cells. From 5 independent experiment it is clear that clones 12H8, 1F10, 1G10, 2D11, 6D11, and 4D2 can induce substantial cell death (Annexin V+PI staining 30% or higher above background), whereas clones 6B2, 15F11, and 8B8 induce almost no cell death in NCI-H358 lung cancer cell line. This data clearly demonstrates that not all anti-EpCAM antibodies posses substantial cytotoxic effect (i.e., having Annexin V+PI staining 30% or higher above background).

### Example 4

### Synergistic Effect of Anti-EpCAM Antibodies with Oxaliplatin

Oxaliplatin, combined with infusional administration of 2 other chemotherapy drugs, 5-fluorouracil/leucovorin (5-FU/LV), is now considered as one of the as first-line treatments for people with advanced carcinoma of the colon or rectum (André et al. (2004) N Engl J Med 350(23):2343-2351). To test the cytotoxicity of anti-EpCAM antibodies in the presence of oxaliplatin, 1×10⁵ COLO 205 cells were seeded to each well of a 96-well plate, and then 0.03, 0.3 and 3 μg/ml of anti-EpCAM or control (9E10) mAbs in combination with oxaliplatin at concentration of 0, 0.01, 0.1, and 1 μg/ml diluted in the medium were added into the cells. After a 24-hr incubation at 37° C., the cells were stained with 0.3 μl of FITC-conjugated Annexin V (Strong Biotech Corp. Cat. No. AVK250) in 50 μl Annexin V binding buffer (Strong Biotech Corp. Cat. No. AVK250) at RT for 20 min. After wash, the cells were then stained with 0.3 μl of propidium iodide in 200 μl Annexin V binding buffer. Annexin V and Propidium Iodide were used as a joint indicator for measuring cell death. The data were acquired with BD FACSCalibur based on an acquisition of 3,000-5,000 cells and analyzed with the Cell Quest software. The combined percentages of the Annexin V+/PI+, Annexin V+/PI− and Annexin V−/PI+populations were considered as dead cells.

TABLE 5
Cytotoxic effect of anti-EpCAM antibodies
in combination with Oxaliplatin in COLO 205 cells.

| mAb concentration tested | | Oxaliplatin concentration tested μg/ml | | | |
|---|---|---|---|---|---|
| μg/ml | | 0 | 0.01 | 0.1 | 1 |
| No mAb added | | 1 | 1 | 1 | 2 |
| 12H8 | 0.03 | 9 | 10 | 9 | 14 |
| | 0.3 | 20 | 23 | 23 | 35 |
| | 3 | 17 | 22 | 26 | 38 |
| 1F10 | 0.03 | −1 | −1 | 1 | 5 |
| | 0.3 | 6 | 10 | 10 | 22 |
| | 3 | 54 | 52 | 57 | 61 |
| 1G10 | 0.03 | 2 | 1 | 2 | 3 |
| | 0.3 | 5 | 4 | 5 | 10 |
| | 3 | 46 | 38 | 44 | 54 |
| 9E10 | 0.03 | −1 | 0 | 1 | 3 |
| (control) | 0.3 | −5 | 4 | −1 | 3 |
| | 3 | 0 | −3 | −1 | 2 |

% of Annexin V + PI staining (untreated background subtracted)

Table 5 summarizes the cell death induced by anti-EpCAM clones (12H8, 1G10 and 1F10) in combination with or without chemodrug oxaliplatin in COLO 205 colon cancer cells. The result demonstrated the synergistic cytotoxic effect of anti-EpCAM antibodies together with oxaliplatin in COLO205 cells, especially at higher concentrations of anti-EpCAM mAbs (0.3 and 3 μg/ml) and oxaliplatin (1 μg/ml).

### Example 5

### Evaluation of Anti-Tumor Effects of Selected Anti-EpCAM Antibodies In Vivo

Whether the in vitro apoptosis-inducing activity of anti-EpCAM antibodies has any in vivo biological relevance was studied in the xenograft model. The antibodies 12H8 (potent cytotoxicity) and 6B2 (minimal cytotoxicity) were studied. 5×10⁶ DLD-1 or 3×10⁶ NCI-H358 cells were implanted subcutaneously into the hind flank region of SCID mice (6-7 weeks) on day 0. Treatment by intraperitoneal injection with antibodies at 12.5 mg/kg for DLD-1 or 30 mg/kg for NCI-H358 in 0.1 ml PBS started on day 1 after tumor-cell inoculation and was repeated at days 4, 8, 11, 15, 18, 22 and 25. As control, 9E10 (an mouse anti-myc antibody) at the same dose were used. Five or six mice were used in each group of the experiment. Tumor growth was assessed based on twice-weekly measurement of tumor volume (mm³) by calipers and the tumor size was calculated using the formula: π/6×larger diameter×(smaller diameter)² (Kievit E, Cancer Research, 60:6649-55). Statistical analysis of tumor growth was performed using the Student's t-test.

TABLE 6(a)
In vivo anti-tumor effect of anti-EpCAM mAbs in colorectal cancer DLD-1 xenograft
(tumor volume Mean ± SD)

| | Day | | | | | | |
|---|---|---|---|---|---|---|---|
| mAb | d29 | d32 | d36 | d39 | d43 | d46 | d49 |
| 9E10 | 1201 ± 284 | 1355 ± 248 | 1665 ± 334 | 1759 ± 349 | 2186 ± 422 | 2606 ± 283 | 3014 ± 276 |
| 6B2 | 947 ± 194 | 1060 ± 140 | 1386 ± 194 | 1541 ± 232 | 1825 ± 341 | 2150 ± 450 | 2425 ± 450 |
| 12H8 | 296 ± 154* | 396 ± 156* | 521 ± 189* | 619 ± 232* | 727 ± 270* | 839 ± 292* | 898 ± 207* |

*t tests P value < 0.05, compared to treatment with control antibody 9E10

TABLE 6(b)
In vivo anti-tumor effect of anti-EpCAM mAbs in lung cancer NCI-H358 xenograft
(tumor volume Mean ± SD)

| | Day | | | | | | |
|---|---|---|---|---|---|---|---|
| | d46 | d50 | d53 | d57 | d60 | d64 | d67 |
| 9E10 | 630 ± 297 | 877 ± 337 | 1249 ± 451 | 1336 ± 468 | 1532 ± 548 | 1809 ± 634 | 1938 ± 610 |
| 6B2 | 539 ± 148 | 724 ± 135 | 862 ± 101 | 1016 ± 182 | 1064 ± 181 | 1198 ± 193 | 1360 ± 240 |
| 12H8 | 325 ± 132* | 475 ± 224* | 597 ± 304* | 656 ± 274* | 680 ± 279* | 859 ± 326* | 1076 ± 464* |

*t tests P value < 0.05, compared to treatment with control antibody 9E10

As shown in Table 6(a) and 6(b), anti-EpCAM antibody 12H8, which has potent cytotoxic effect in vitro, efficiently inhibited colorectal (DLD-1) and lung (NCI-H358) tumor growth, whereas antibody 6B2 had much less inhibitory effect.

### Example 6

### Epitope Mapping

(1) Generation of Mutated Domain I of hEpCAM

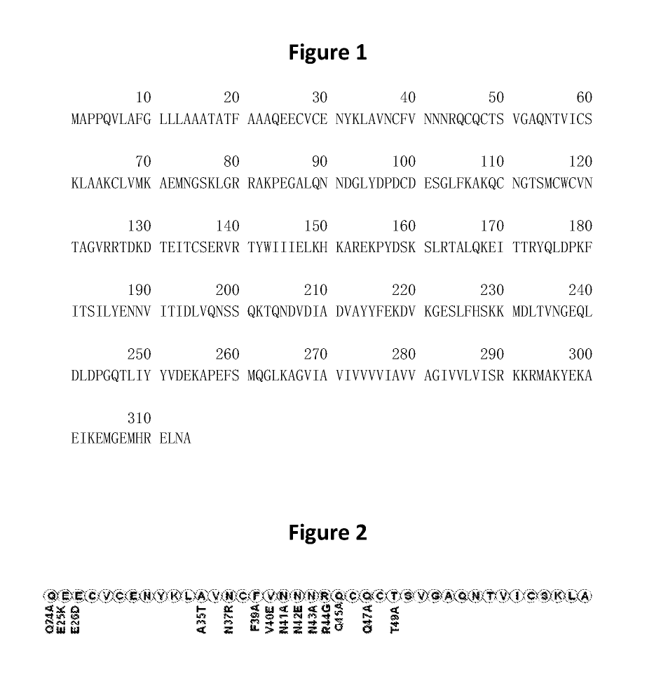

The cDNA encoding the sequence covering the first EGF-like repeat (aa 1-63) of human EpCAM (EGF-I) was amplified by PCR. For facilitating the purification of expressed recombinant protein, this domain was expressed as fusion proteins with the constant region of human immunoglobulin gamma 1 heavy chain (EGFI-Cr1) in pVac4A1ΔSP vector. Overlapping PCR and PCR-based site-directed mutagenesis were used to introduce mutation shown in FIG. 2 into the wild-type of EGF-I domain. The plasmids expressing wild type or mutant EpCAM-Cr1 fusion protein were transiently transfected into COS-7 cells by Lipofectamine 2000 (Invitrogen, Cat. No. 11668) according to the manufacturer's instruction. The transfected cells were grown in DMEM medium, containing 10% FBS Ultra low-Ig (Invitrogen, Cat. No. 16250) for 5 days before the culture medium was collected for purification of the wild-type and mutated EGFI-Cr1 recombinant proteins using protein G Sepharose (Amersham Bioscience).

### (2) Binding of Anti-EpCAM Clones to Wild-Type and Mutant EpCAM Protein

A direct ELISA was used for testing the reactivity of anti-EpCAM antibodies toward variant EpCAM mutants. Briefly, purified EpCAM EGF-1 fusion proteins (WT, Q24A, E25K, E26D, A35T, N37R, F39A, V40E, N41A, N42E, N43A, R44G, Q45A, Q47A or T49A) and irrelevant control (CEA-Cr1) were pre-diluted to 0.5 μg/ml in coating buffer (8.4 g NaHCO₃, 3.4 g Na₂CO₃ in 1 L H₂O, pH 9.5) and aliquoted (100 μl/well) into 96-well plates. After overnight incubation at 4° C., the wells were blocked with 1% of BSA in PBS (200 μl/well) for 2-hr at RT, followed by incubation sequentially with 0.1 μg/ml primary antibody (anti-EpCAM clones or 9E10) and 1:5,000-diluted corresponding Peroxidase-conjugated secondary antibodies (Goat anti-mouse IgG(H+L) from Southern Biotech, Cat. No. 1031-05 and Goat anti-human IgG(H+L) from Jackson ImmunoResearch, Cat. No. 109-035-088) for 1-hr at RT. Plates were then washed 3 times with PBST followed by the addition of the enzyme substrate TMB (BD Biosciences, Cat. No. 555214). After the suggested incubation time, 2N H₂SO₄ was added (50 μl per well) for stopping the reaction. The optical densities at 450 nm wavelength were measured on an ELISA plate reader.

TABLE 7(a)
Relative binding activities of anti-EpCAM clones to mutant EpCAM compared to wild-type EpCAM.

| mAb | WT | Q24A | E25K | E26D | A35T | N37R | F39A | V40E | N41A | N42E | N43A | R44G | Q45A | Q47A | T49A |
|---|---|---|---|---|---|---|---|---|---|---|---|---|---|---|---|
| 12H8 | 100 | 0 | 1 | 94 | 95 | 94 | 96 | 93 | 81 | 23 | 96 | 91 | 89 | 93 | 94 |
| 1G10 | 100 | 44 | 3 | 1 | 89 | 1 | 78 | 71 | 23 | 93 | 96 | 95 | 80 | 49 | 17 |
| 1F10 | 100 | 93 | 10 | 98 | 93 | 97 | 81 | 1 | 63 | 95 | 100 | 1 | 84 | 92 | 94 |
| 2D11 | 100 | 99 | 93 | 97 | 95 | 94 | 97 | 93 | 12 | 58 | 5 | 7 | 89 | 96 | 95 |
| 6D11 | 100 | 47 | 4 | 57 | 39 | 69 | 23 | 2 | 2 | 83 | 65 | 2 | 8 | 33 | 67 |
| 4D2 | 100 | 41 | 2 | 52 | 27 | 64 | 11 | 1 | 0 | 82 | 57 | 1 | 4 | 23 | 63 |

The binding of each anti-EpCAM antibody to individual EpCAM mutant (FIG. 2) was studied and compared to its binding to wild type EpCAM molecule. The number in Table 7(a) represents the percentage of binding activities of each antibody to each mutant protein compared to its binding to wild-type (WT) protein (which is set as 100%). Each number represents an average of data from two (for 12H8) or three (for 1G10, 1F10, 2D11, 6D11, 4D2) independent ELISA experiments.

TABLE 7(b)
The essential residues for anti-EpCAM antibody binding

| mAb | Essential residues |
|---|---|
| 12H8 | Q24, E25 and N42 |
| 1G10 | Q24, E25, E26, N37, N41, Q47 and T49 |
| 1F10 | E25, V40 and R44 |
| 2D11 | N41, N43, and R44 |
| 6D11 | Q24, E25, A35, F39, V40, N41, R44, Q45, and Q47 |
| 4D2 | Q24, E25, A35, F39, V40, N41, R44, Q45, and Q47 |

The amino acids in the EGF-I domain (aa24-63) whose mutations cause more than 50% reduction in the relative binding activity are considered as “essential” residues for the antibody binding. Table 7(b) summarizes the essential residues for each antibody clone.

### Example 7

### Cloning of the Variable Regions of Light and Heavy Chains of 12H8, 1G10, 1F10, 2D11, 4D2, and 6D11

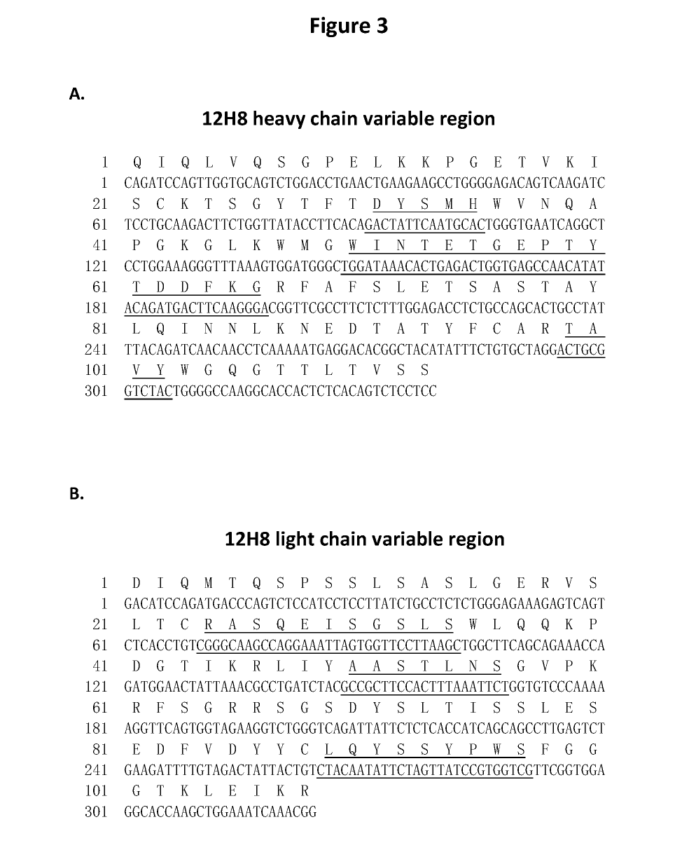

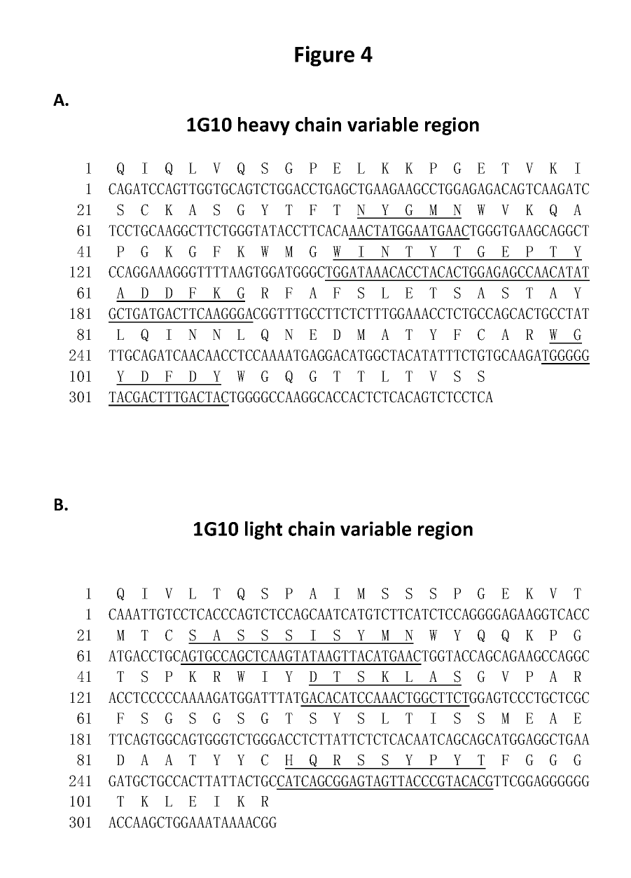

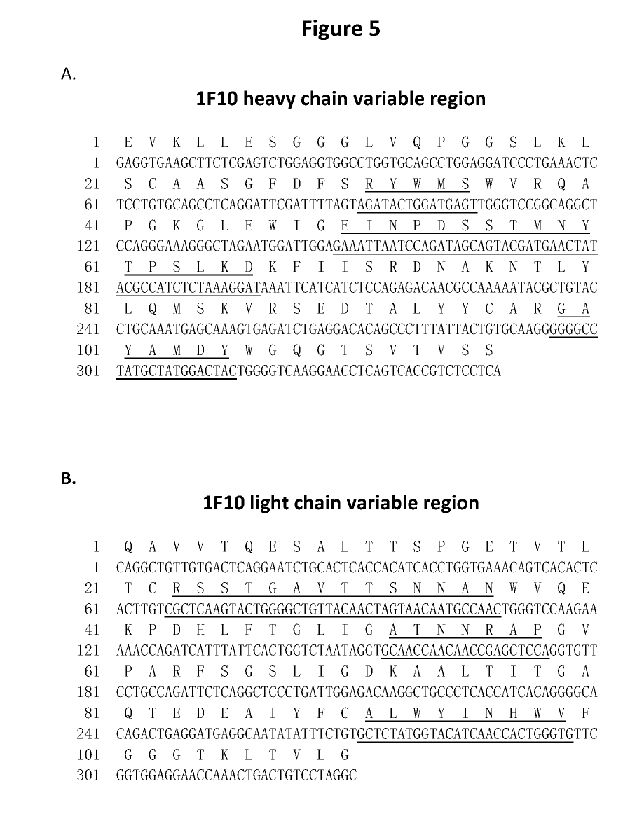

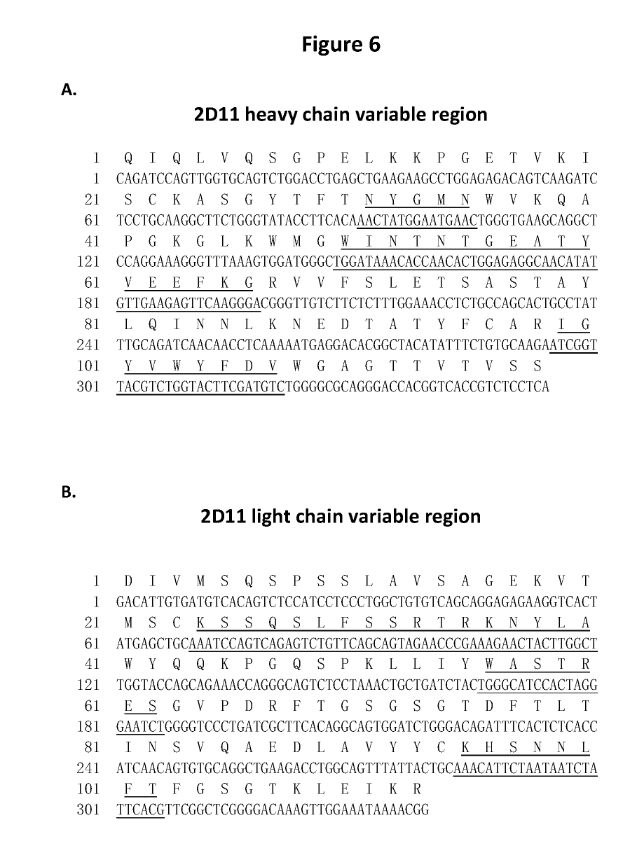

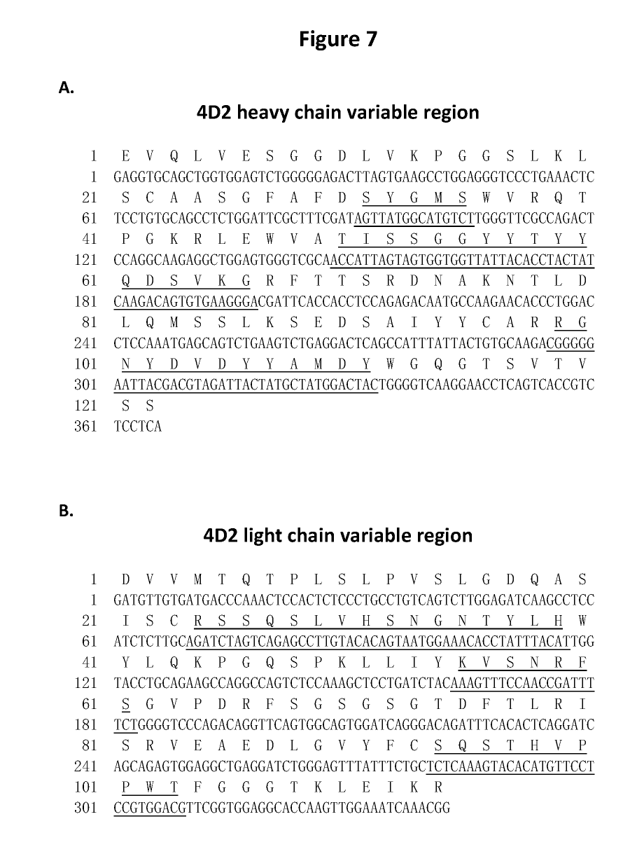

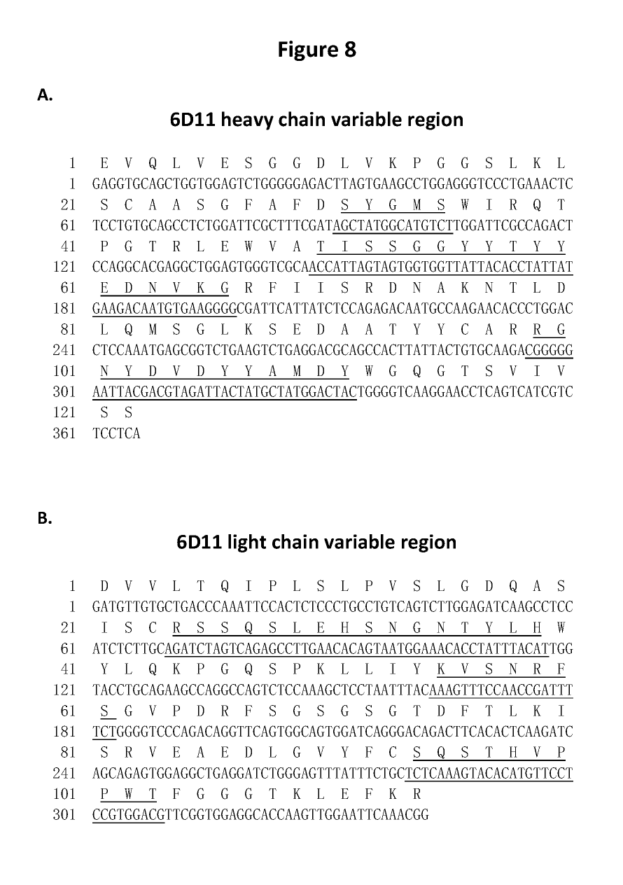

The cDNA for variable regions (V region) of 12H8, 1G10, 1F10, 2D11, 4D2, and 6D11 light and heavy chains were amplified by PCR, and subcloned into pCR11-Blunt-TOPO (Invitrogen) for sequence determination. Nucleotide sequences were obtained from several independent clones and analyzed. The mature amino acid sequences of the light and heavy chain V regions of 12H8 (IgG₂ₐ, K), 1G10 (IgG₁, K), 1F10 (IgG₁, λ1), 2D11 (IgG₁, K), 4D2 (IgG₁, K), and 6D11 (IgG₁, K) and the Kabat CDRs were identified as shown in FIGS. 3-8. Constant region sequences of mouse immunoglobulin IgG1 (Honjo et al., Cell 18:559-568, 1979), IgG2a (Olio et al., Proc Natl Acad Sci USA. 78(4):2442-2446, 1981), Kappa light chain (Hieter et al. Cell 22(1 Pt 1):197-207, 1980) and Lambda 1 light chain (Selsing et al., Proc Natl Acad Sci USA. 79:4681-4685, 1982) isotype have been described.

### Example 8

### Evaluation of Anti-EpCAM and Anti-CD3 Bispecific Antibodies (“Anti-EpCAM×Anti-CD3 bsAbs”)

### Materials and Methods

Humanization of Anti-EpCAM Antibody and the Generation of EpCAM-Specific Arm of Single-Chain Fragments of Variable Region (scFv)

Complementarity-determining region (CDR) grafting was used to generate the variable region of humanized 12H8B (h12H8B), 12H8Cc.2 (h12H8C) and h2D11B.

For h12H8Cc.2, BLASTP searches against the entire non-redundant Genebank database was used to identify human antibodies which shares the most sequences identity/similarity with m12H8. The CDRs of murine 12H8 heavy chain were incorporated into the framework sequences of human antibody AAA17956 (Genebank no. AAA17956) heavy chain variable region, which has 66.7% sequences identity with murine 12H8 heavy chain. The CDRs of murine 12H8 light chain were incorporated into the framework sequences of human antibody AAA86778 (Genebank no. AAA86778) light chain variable region, which has 69.2% sequences identity with murine 12H8 light chain.

For h2D11B, the sequences of the variable regions of murine 2D11 were compared to a data base consisting of sequences of murine antibodies already humanized, in order to find the murine antibody with most sequence identity/similarity with murine 2D11, and the corresponding humanization framework sequences. As a consequence, the CDRs of murine 2D11 heavy chain was incorporated into the framework sequences of human antibody VHIII heavy chain variable region (the acceptor antibody for murine antibody A4.6.1 (Baca M. et al., J. Biol. Chem. 1997, 272:10678-10684.), of which the heavy chain sequences showed most identity/similarity with the heavy chain of murine 2D11), and the CDRs of murine 2D11 light chain was incorporated into the frame work sequences of human antibody REI (Verhoeyen M E et al., Immunology 1993, 78:364-370.) (the acceptor antibody for murine antibody HMGF1, of which the light chain showed most sequence identity/similarity with the light chain of murine 2D11).

Similar approach was used to generate h12H8B, for which the CDRs of murine 12H8 were incorporated into the framework sequences of human heavy chain subgroup 3 (V_{H} III) and human kappa 1 (V_{L}k1) variable regions (Studnicka G M et al., Protein Eng. 1994, 7(6):805-14).

Nucleotides were synthesized to generate a humanized 12H8 and 2D11 versions. The assembled V_{H} and V_{L} fragments were then inserted into pcDNA5-FRT-hIgG1κ vector. The assembled expression plasmid h12H8B/pcDNA5-FRT-hIgG1κ, h12H8Cc.2/pcDNA5-FRT-hIgG1κ and h2D11B/pcDNA5-FRT-hIgG1κ containing both the heavy chain and light chain gene of humanized antibody, were used to express h12H8B, h12H8C and h2D11B antibody, respectively for functional study. The V_{H} and V_{L} domains of h12H8B, h12H8Cc.2 and h2D11B were used to generate EpCAM-specific arm of single-chain fragments of variable region (scFv).

Construction and Expression of AbGn bsAbs

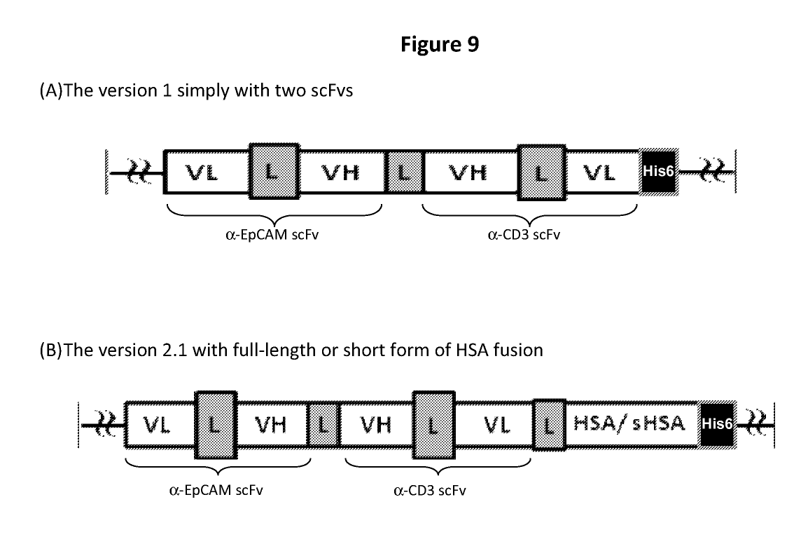

A panel of anti-EpCAM×anti-CD3 bsAbs (FIG. 9 & Table 8) were constructed by fusion of two single-chain fragments of variable region (scFv) through a 5-amino-acid linker (L₅).

TABLE 8
Anti-EpCAMxanti-CD3 constructs

| | | Domain | HSA |
|---|---|---|---|
| Construct name | Construct description | arrangement | fusion |
| h2D11B-v1 | h2D11Bxanti-CD3 | V_{L}-V_{H}-V_{H}-V_{L}-His₆ | No |
| h2D11B-v2.1 | h2D11Bxanti-CD3xHSA | V_{L}-V_{H}-V_{H}-V_{L}- | Full- |
| | | HSA-His₆ | length |
| h12H8B-v1 | h12H8Bxanti-CD3 | V_{L}-V_{H}-V_{H}-V_{L}-His₆ | No |
| h12H8B-v2.1 | h12H8Bxanti-CD3xHSA | V_{L}-V_{H}-V_{H}-V_{L}- | Full- |
| | | HSA-His₆ | length |
| h12H8C-v1 | h12H8Cxanti-CD3 | V_{L}-V_{H}-V_{H}-V_{L}-His₆ | No |
| h12H8C-v2.1 | h12H8Cxanti-CD3xHSA | V_{L}-V_{H}-V_{H}-V_{L}- | Full- |
| | | HSA-His₆ | length |
| h12H8C-v2.1- | h12H8Cxanti- | V_{L}-V_{H}-V_{H}-V_{L}- | Short- |
| sHSA | CD3xsHSA | sHSA-His₆ | form |

EpCAM-specific single-chain Fv fragment was derived from V_{H} and V_{L} domains of h12H8B, h12H8Cc.2 and h2D11B. A 15-amino-acid linker (L₁₅) was inserted between the V_{L} and V_{H} domains to form the scFv. All candidates shared the same T cell-specific arm of scFv derived from a known mouse monoclonal antibody against human CD3 epsilon (CD3ε). For generation of anti-EpCAM×anti-CD3 bsAbs, the 3′ end of anti-EpCAM arm V_{H} was linked to the 5′ end of CD3ε arm V_{H} through a 5-amino-acid linker (L₅). A 6×His sequence was incorporated 3′ to CD3ε arm for later affinity purification. This bsAb consisting of only two scFvs was named version 1 (v1). For the version 2.1 construct, an additional cDNA which encodes either a full-length (NCBI Reference Sequence: NM_{—}000477.5) or partial human serum albumin (HSA or sHSA) (Müller D et al., J Biol Chem 2007, 282: 12650-12660) was inserted between the CD3ε arm of scFv and 6×His sequence. The 3′ end of CD3ε arm V_{L} was linked to the 5′ end of HSA/sHSA through a 5-amino-acid linker (L₅). The construct with full length human serum albumin was named h12H8C-v2.1, and the construct with partial human serum albumin was named h12H8C-v2.1-sHSA.

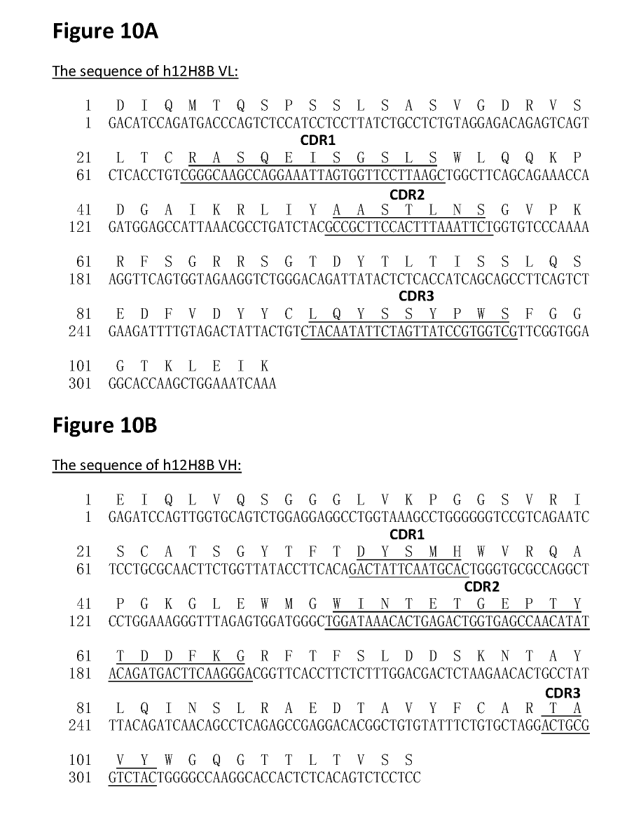

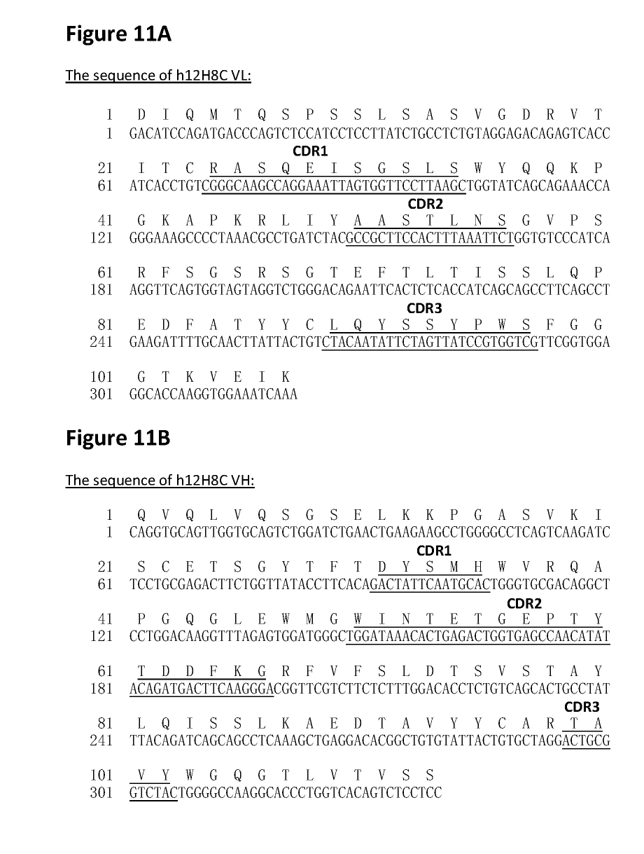

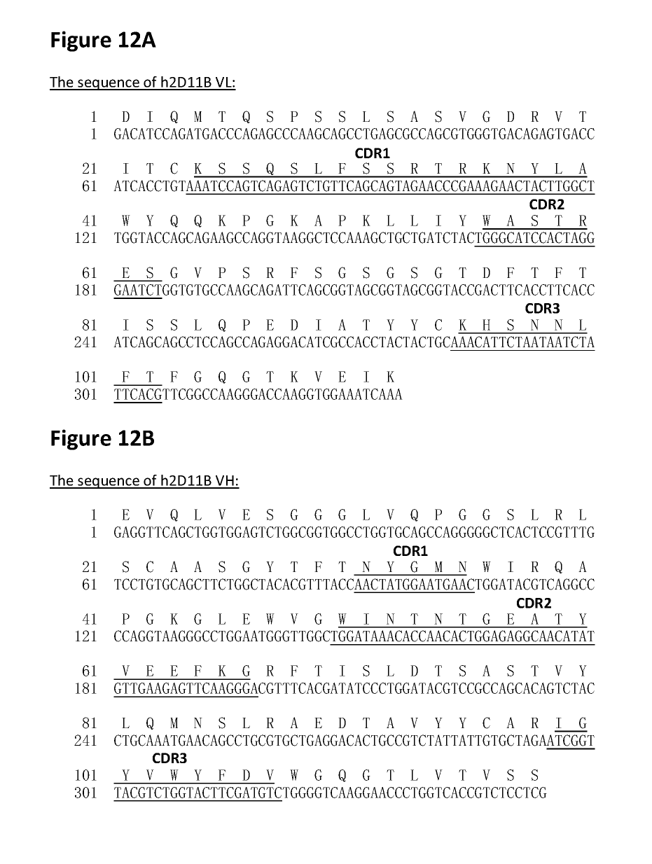

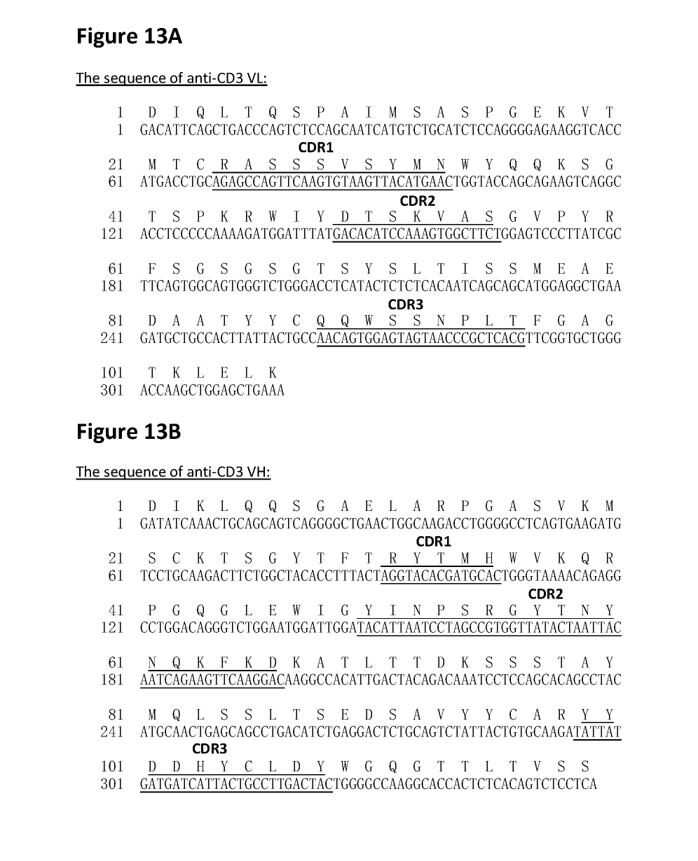

The sequence for h12H8B V_{L} (SEQ ID NO:29 for amino acid sequence; SEQ ID NO:30 for nucleic acid sequence) is shown in FIG. 10A. The sequence for h12H8B V_{H} (SEQ ID NO:27 for amino acid sequence; SEQ ID NO:28 for nucleic acid sequence) is shown in FIG. 10B. The sequence for h12H8C V_{L} (SEQ ID NO:33 for amino acid sequence; SEQ ID NO:34 for nucleic acid sequence) is shown in FIG. 11A. The sequence for h12H8C V_{H} (SEQ ID NO:31 for amino acid sequence; SEQ ID NO:32 for nucleic acid sequence) is shown in FIG. 11B. The sequence for h2D11B V_{L} (SEQ ID NO:37 for amino acid sequence; SEQ ID NO:38 for nucleic acid sequence) is shown in FIG. 12A. The sequence for h2D11B V_{H} (SEQ ID NO:35 for amino acid sequence; SEQ ID NO:36 for nucleic acid sequence) is shown in FIG. 12B. The sequence for anti-CD3 V_{L} (SEQ ID NO:57 for amino acid sequence; SEQ ID NO:58 for nucleic acid sequence) is shown in FIG. 13A. The sequence for anti-CD3 V_{H} (SEQ ID NO:55 for amino acid sequence; SEQ ID NO:56 for nucleic acid sequence) is shown in FIG. 13B.

The sequence for linker (L₁₅) (SEQ ID NO:49 for amino acid sequence; SEQ ID NO:50 for nucleic acid sequence) inserted between the V_{L} and V_{H} of anti-EpCAM scFv is shown below:

| | |
|---|---|
| 1 | G G G G S G G G G S G G G G S |
| 1 | GGTGGTGGTGGTTCTGGCGGCGGCGGCTCCGGTGGTGGTGGTTCT |

The sequence for linker (L₅) (SEQ ID NO:51 for amino acid sequence; SEQ ID NO:52 for nucleic acid sequence) inserted between the two scFvs is shown below:

| | |
|---|---|
| 1 | G G G G S |
| 1 | GGTGGAGGCGGATCC |

The sequence for linker (SEQ ID NO:53 for amino acid sequence; SEQ ID NO:54 for nucleic acid sequence) inserted between the V_{H} and V_{L} of anti-CD3 scFv is shown below:

| | |
|---|---|
| 1 | V E G G S G G S G G S G G S G G V D |
| 1 | GTCGAAGGTGGAAGTGGAGGTTCTGGTGGAAGTGGAGGTTCAGGTGGAGTCGAC |

The linker (L₅) was used between the anti-CD3 scFv and HSA/sHSA.

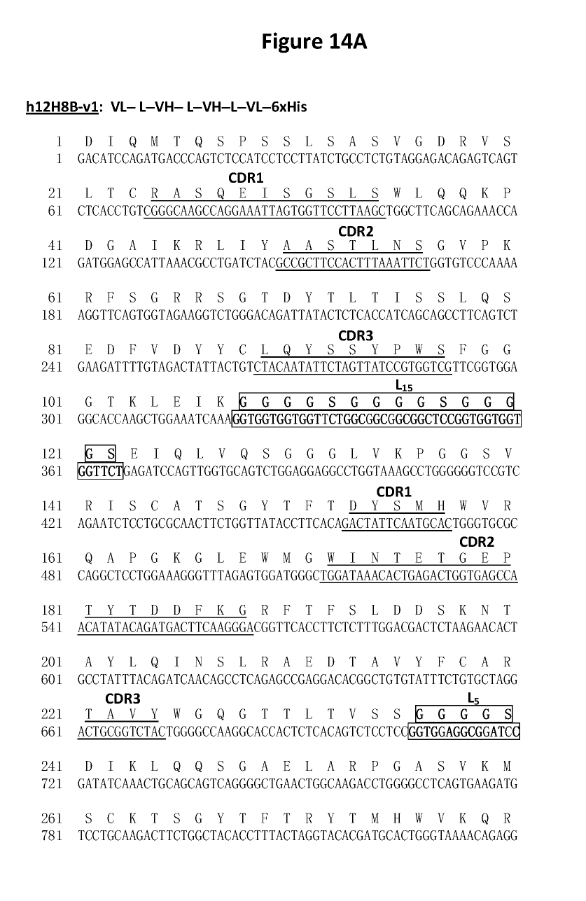

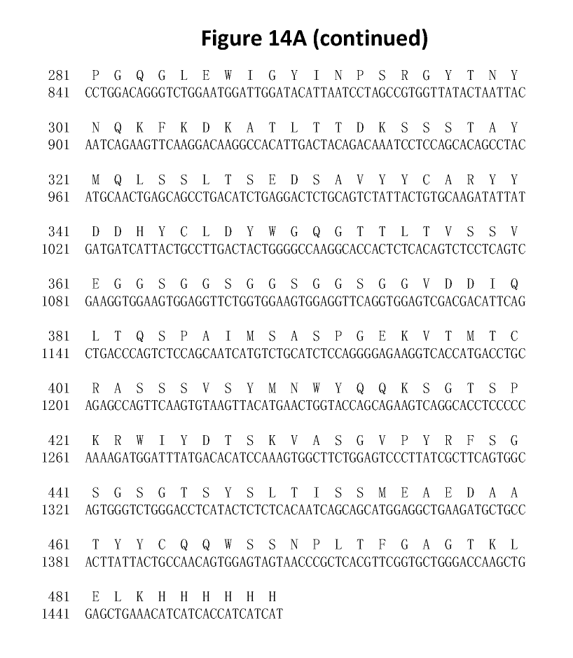

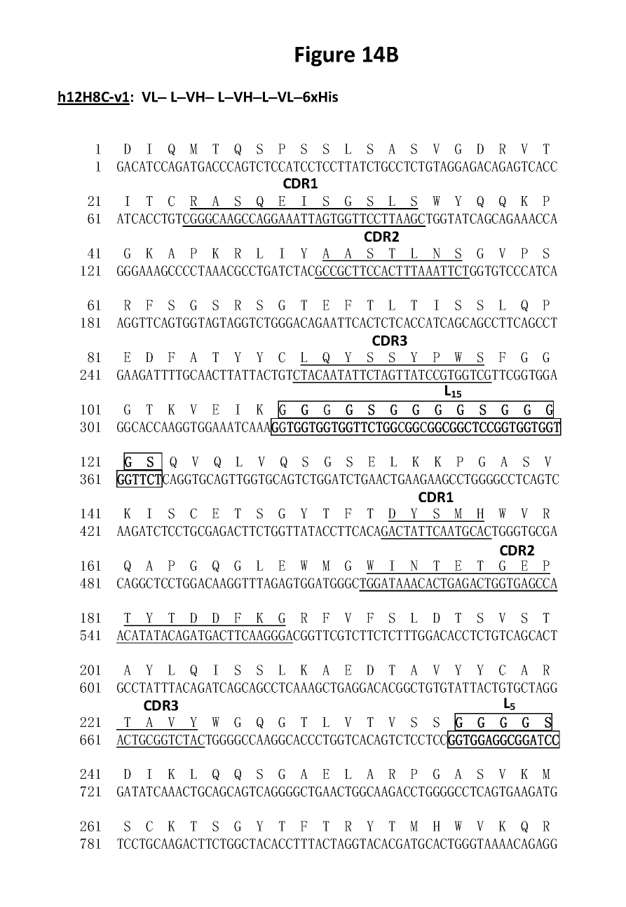

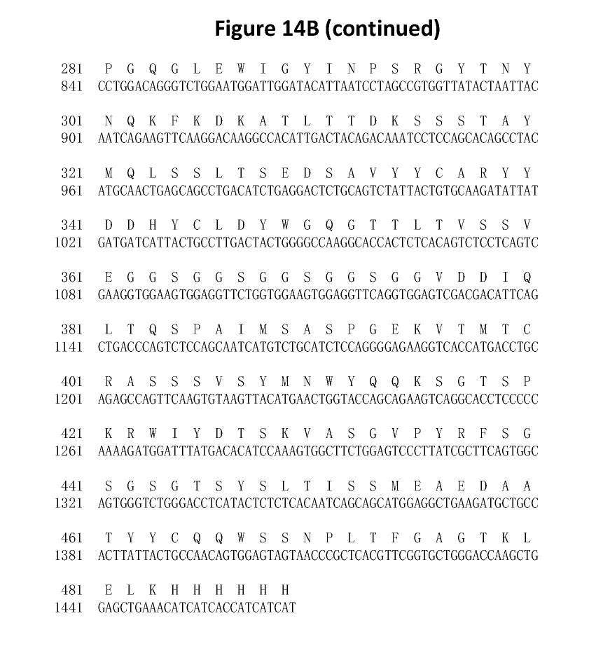

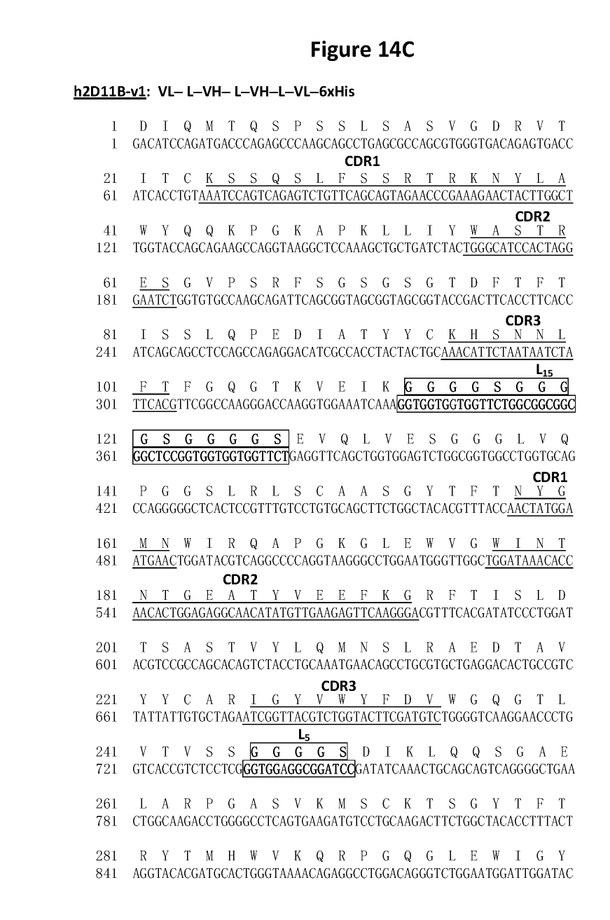

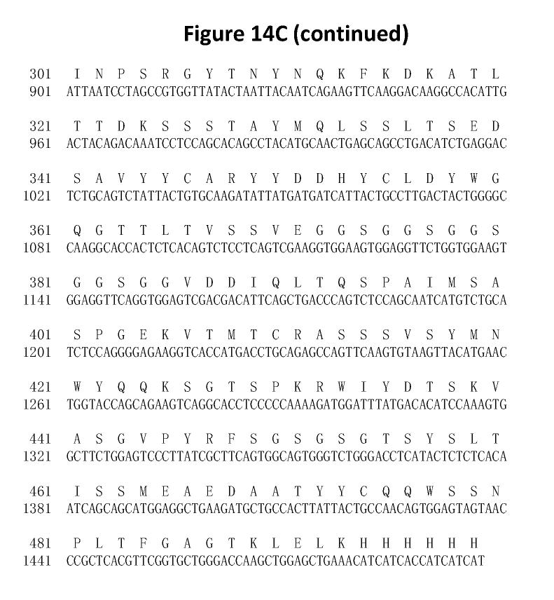

The sequence for v1 version h12H8B bsAb (“h12H8B-v1”) (SEQ ID NO:39 for amino acid sequence; SEQ ID NO:40 for nucleic acid sequence) is shown in FIG. 14A. The sequence for v1 version h12H8C bsAb (“h12H8C-v1”) (SEQ ID NO:41 for amino acid sequence; SEQ ID NO:42 for nucleic acid sequence) is shown in FIG. 14B. The sequence for v1 version h2D11B bsAb (“h2D11B-v1”) (SEQ ID NO:43 for amino acid sequence; SEQ ID NO:44 for nucleic acid sequence) is shown in FIG. 14C.

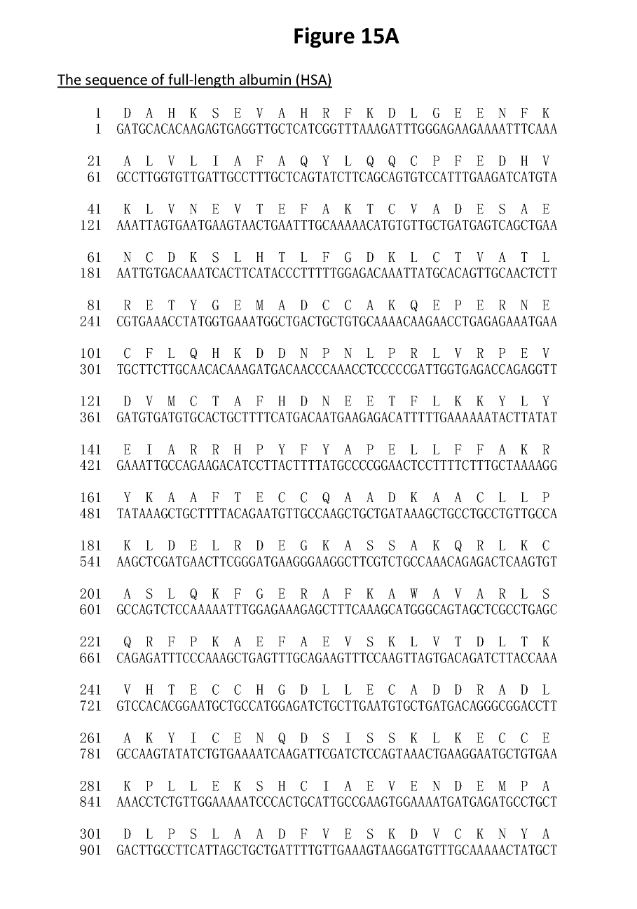

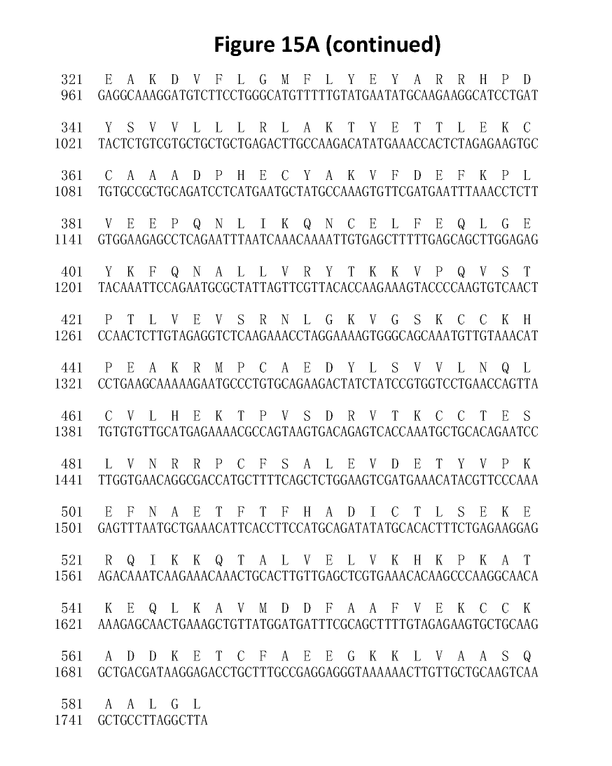

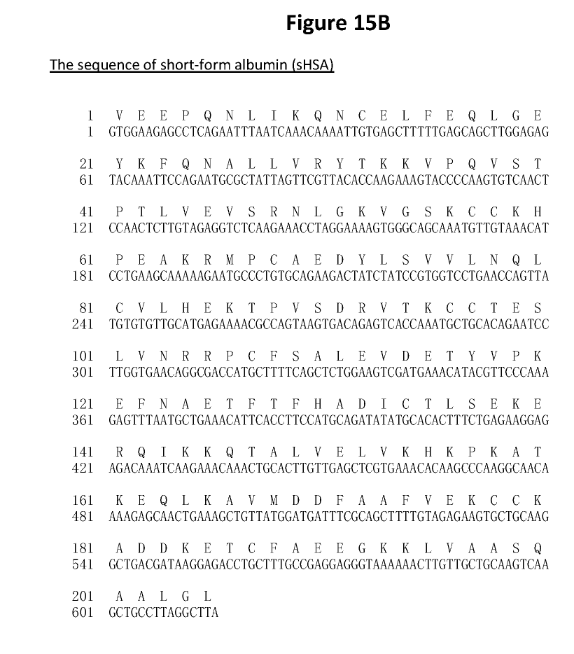

The sequence for full-length albumin (SEQ ID NO:45 for amino acid sequence; SEQ ID NO:46 for nucleic acid sequence) used for bsAb fusion is shown in FIG. 15A. The sequence for short-form albumin (SEQ ID NO:47 for amino acid sequence; SEQ ID NO:48 for nucleic acid sequence) used for bsAb fusion are shown in FIG. 15B.

The constructs for bsAbs further contained a signal peptide sequence at the N-terminus, such as signal peptide 1 (SEQ ID NO:59 for amino acid sequence; SEQ ID NO:60 for nucleic acid sequence) or signal peptide 2 (SEQ ID NO:61 for amino acid sequence; SEQ ID NO:62 for nucleic acid sequence). The sequences for signal peptides are shown below:

| Signal peptide 1: | |
|---|---|
| 1 | M K L P V R L L V L M F W I P V S S S |
| 1 | ATGAAATTGCCTGTTAGGCTGTTGGTGCTGATGTTCTGGATTCCTGTTTCCAGCAGT |
| Signal peptide 2: | |
| 1 | M E T D T L L L W V L L L W V P G S T G |
| 1 | ATGGAGACAGACACACTCCTGCTATGGGTGCTGCTGCTCTGGGTTCCAGGTTCCACCGGT |

The assembled sequences were cloned into expression vector pcDNA5-FRT (Invitrogen) and transfected into mammalian Chinese hamster ovary (CHO) cells. The culture supernatants containing bsAb were collected for further purification and functional study.

Purification of AbGn bsAbs

The cell culture supernatant fluid that contained the secreted bsAb was collected from 100% confluent CHO cell cultures. The bsAb was extracted from the supernatant fluid through its C-terminal 6×His tail using immobilized metal-affinity chromatography technology with the nickel-charged Ni-NTA resin (Chelating Sepharose Fast Flow, GE). The eluted protein product was buffer-exchanged into phosphate buffer containing 150 mM NaCl in 25 mM phosphate buffer, pH 7.2 and concentrated using the Amicon Ultra-10 (Millipore). The concentrated protein was then applied for gel filtration on a HiLoad 16/60 Superdex 200 column (Amersham) to get monomeric bispecific Abs. The final products were filter-sterilized through a 0.2-μm filter before use.

### Cell Lines and Culture

Human pancreatic cancer cell Panc 02.03, lung cancer cell NCI-H358 and Multiple Myeloma cell RPMI 8226 were obtained from the American Type Culture Collection (Manassas, Va., USA). Human colorectal cancer cell DLD-1 was purchased from Food Industry Research and Development Institute, Hsin-chu, Taiwan.

NCI-H358, RPMI 8226 and DLD-1 were maintained in RPMI 1640 medium (GIBCO BRL) containing 1.5 g/L sodium bicarbonate, 4.5 g/L glucose, 10 mM HEPES, and 1.0 mM sodium pyruvate, and supplemented with 10% FBS, 100 units/ml of penicillin and 100 μg/ml of streptomycin (GIBCO BRL). Panc 02.03 was cultured in the same modified RPMI 1640 medium (GIBCO BRL) with 15% FBS (Hyclone), 100 units/ml of penicillin and 100 μg/ml of streptomycin (GIBCO BRL). The stable cell line CHO/EpCAM was maintained in Ham's F12 medium, containing 10% FBS and 600 μg/ml Hygromycin B. Cells were all cultured at 37° C. in a humidified atmosphere with 5% CO₂.

FACS Analysis for bsAb Binding Assay

bsAbs binding to human CD3 was tested on human lymphocytes purified by ficoll density centrifugation. A human EpCAM stably transfected cell line CHO/EpCAM was used to test the binding of bsAbs to human EpCAM. 2×10⁵ cells were seeded in wells of a 96-well, v-bottomed plate and incubated with un-diluted culture supernatant at 4° C. for 30 min. Cells were then washed twice with FACS buffer (1×PBS+1% FBS). PE-conjugated anti-6×His Tag antibody (AD1.1.10, Abcam) was added and cells were incubated at 4° C. for 30 min. Cells incubated with no bsAb-containing medium and stained with anti-6×His Tag PE-conjugated antibody were served as a negative control (2^{nd} only). Cells were then washed 2 times with FACS buffer and analyzed by flow cytometry for bsAb binding.

### FACS-Based Cytotoxicity Assay

Human peripheral blood mononuclear cells (“PBMC”) were prepared by Ficoll density centrifugation from whole blood samples of healthy donors and used as effector cells in the assay.

Target cells were labeled with the carboxyfluorescein diacetate succinimidyl ester (CFSE) (Invitrogen, Catalog No. C1157) or fluorescent membrane dye PKH-26 (Sigma-Aldrich, Catalog No. PKH26GL) according to the manufacturer's instructions. In brief, target cells were washed once in PBS or serum-free medium, and adjusted the cell concentration to 1×10⁶ per ml in PBS containing 0.1% BSA for CFSE labeling and 5×10⁶ per ml in Diluent C for PKH-26 labeling.

For CFSE labeling, equal volume (1V) of target cells suspension and 2.5 μM CFSE working solution were added together and immediately mixed gently. Cells were labeled for 5 min at 37° C. before the reaction was stopped by adding 10 ml of complete medium (containing 10% FBS). For PKH-26 labeling, a 2×PKH-26 dye working solution (4×10⁻⁶ M) was prepared by adding 4 μl of PKH-26 ethanolic dye to 1 ml Diluent C. Equal volume (1V) of target cells suspension and 2×PKH-26 dye working solution were added together and immediately mixed by pipetting. Cells were labeled for 5 min with periodic mixing before the reaction was stopped by adding an equal volume of FBS (2V).

After three washing steps with complete culture medium, labeled target cells were counted and mixed with effector cells at an effector-to-target ratio of 10:1 or 5:1 in complete culture medium. Target (1.5×10⁴) and effector cells (7.5×10⁴ to 1.5×10⁵) in a volume of 50 cell medium were added per well in a 96-well flat-bottomed plates. Fifty microliter of purified bsAb or culture supernatant (culture sup.) at indicated concentrations or of complete medium for an untreated control were added to the wells. Cells were incubated at 37° C. in a 5% CO₂ humidified incubator for 18-20 hr. The cells were then harvested and stained with 50 μl FITC-conjugated Annexin V (Strong Biotech Corp. Cat. No. AVK250; 0.3 μl stock in 50 μl Annexin V binding buffer) for 15-20 min at room temperature (“RT”). After wash, the cells were then stained with 50 μl of propidium iodide (PI, 0.3 μl stock in 50 μl Annexin V binding buffer) and analyzed by flow cytometry (BD LSR, BD Life Sciences). PKH-26-positive cells was gated for analysis and the dead cell percentage were calculated by combining percentages of Annexin V−/PI+, Annexin V+/PI+ and Annexin V+/PI-populations. For CFSE-labeled assay, the step of Annexin V staining was omitted, and CFSE-positive cells was gated for analysis and the dead cell percentage were calculated with PI+ population only.

### Cell Growth Inhibition Assay

BsAb-mediated cancer cell killing was measured by a colorimetric WST-based cell proliferation assay. Peripheral blood mononuclear cells (PBMC) (6×10⁶ cells/ml) from healthy donors were isolated by ficoll density centrifugation and subsequently mixed with equal volume of target cancer cells (1.2×10⁶ cells/ml) in completed culture medium. Fifty microliter aliquots of the cell mix containing 3×10⁴ target cells and 1.5×10⁵ effector cells were added in a 96-well flatten-bottomed plate. Fifty microlitre of purified bsAb or culture supernatant at indicated concentrations or complete medium for an untreated (UT) control were added to the corresponding well. After 2 days (40-44 hr) of co-cultivation at 37° C., 5% CO₂, PBMC, which were non-adherent, were removed by two times PBS washing. Viable adherent cancer cells were incubated with WST-1 solution (10 μl Cell Proliferation Reagent WST-1 (Roche) in 100 culture medium) at 37° C. until the absorbance at OD450 nm for the untreated control samples reached 1.5-2.5 (around 2 hr). At least duplicates were performed in all experiments and the absorbance was averaged. The percentage of cell growth inhibition was calculated as follows:

the percentage of cell growth inhibition=(UT_{OD450}−Sample_{OD450})/UT_{OD450}×100%.

### DLD-1 Human Colon Carcinoma Xenograft

Six to Seven-week-old, female, non-obese diabetic severe-combined immunodeficient (NOD-SCID) mice (Biolasco) were used for in vivo studies. These mice carry the NOD and SCID mutation, which causes a deficiency of T cells, B cells, natural killer cells (NK) and macrophages. Human effector cells were isolated from whole blood samples of healthy donors. Peripheral blood mononuclear cells (PBMC) were prepared by Ficoll density centrifugation. DLD-1 cancer cells 5×10⁶ in 0.1 mL culture medium were inoculated subcutaneously into the left/right flank of the mice. After 8 days for outgrowth, when tumors measured approximately 50-200 mm³, as calculated by the formula volume=[length)×(width)²]÷2, mice were administered 5×10⁶ hPBMC as effector cells by intratumoral (“IT”) injection and divided into the following groups:

For the 3 treatment groups, 8 animals per group were intravenously (“iv”) treated with bsAb at the doses of 10 μg/mouse of h12H8C-v2.1, h12H8C-v2.1-sHSA or the vehicle (PBS) 1 hr after hPBMC IT injection and treatment was repeated for 8 consecutive days. An additional group of 4 animals was kept as DLD-1 cells only to evaluate nonspecific effects induced by the hPBMC effector cells (data not shown). The progress of each tumor was monitored 2 times per week until average tumor size reached a volume around 1.5 cc, at that point the mice were sacrificed. Average of tumor sizes was presented for indicated time for comparison among each treatment group. Statistical comparison was performed by the Student t test for paired data.

### Results

Binding of AbGn Constructed bsAbs to Target Cells

Binding of anti-EpCAM×anti-CD3 bsAbs to EpCAM-positive and CD3-positive cells was studied by FACS analysis. BsAbs binding to human CD3 was tested on human lymphocytes prepared from two donors (Donor N095 and Donor N094). See Table 9A. A human EpCAM stably transfected cell line CHO/EpCAM was used to test the binding of bsAbs to human EpCAM. See Table 9B. The results showed that h2D11B-v1, h2D11B-v2.1, h12H8B-v2.1 and h12H8C-v2.1 were able to bind to human lymphocytes and CHO/EpCAM cells (Tables 9A & 9B).

TABLE 9
Binding of AbGn constructed anti-EpCAM × anti-CD3 bsAbs to human lymphocytes
from two different donors (A) and EpCAM expressed CHO cells (CHO/EpCAM) (B).
(A) CD3-arm binding. Percentage (%) of binding positive lymphocytes in hPBMC is shown
in table from left to right for each individual bsAbs in un-diluted culture supernatant.

| | | | Anti-His | | | | |
|---|---|---|---|---|---|---|---|
| Control or bsAb- | | Un- | (2^{nd} | h2D11B- | h2D11B- | h12H8C- | h12H8B- |
| containing culture sup. | | stained | only) | v1 | v2.1 | v2.1 | v2.1 |
| Gated | Donor | 1% | 2% | 77% | 73% | 55% | 58% |
| Lymphocytes | N095 | | | | | | |
| | Donor | 1% | 1% | 68% | 65% | 47% | 51% |
| | N094 | | | | | | |

Cells incubated with no bsAb-containing medium and stained with anti-6xHis Tag PE-
conjugated antibody were served as a negative control (2nd only). The results showed that
h2D11B-v1, h2D11B-v2.1, h12H8B-v2.1 and h12H8C-v2.1 were able to bind to human
lymphocytes.

(B) EpCAM-arm binding. Mean florescent intensity (MFI) is shown in table from left
to right for each individual bsAbs in un-diluted culture supernatant.

| Control or bsAb- | | Anti-His | | | | |
|---|---|---|---|---|---|---|
| containing culture | Un- | (2^{nd} | h2D11B- | h2D11B- | h12H8C- | h12H8B- |
| sup. | stained | only) | v1 | v2.1 | v2.1 | v2.1 |
| CHO/EpCAM | 5 | 4 | 1701 | 1871 | 1801 | 1555 |

Cells incubated with no bsAb-containing medium and stained with anti-6xHis Tag PE-
conjugated antibody were served as a negative control (2nd only). The results showed that
h2D11B-v1, h2D11B-v2.1, h12H8B-v2.1 and h12H8C-v2.1 were able to bind to EpCAM

In Vitro Cytotoxic Efficacy of AbGn bsAbs with Human T Lymphocytes

The specificity of bsAb was investigated with CHO cell lines with or without human EpCAM expression. Effector cells (human PBMC) were mixed with target cells at an effector-to-target ratio of 10:1 and incubated with serial dilutions of culture sup. (2×, 6×, and 20×). Target cell death (indicated by positive propidium iodide staining) was determined by flow cytometry after 20 h of incubation (Table 10). Whereas virtually no cell death was observed for parental CHO cells in the presence of T cells and bsAb, EpCAM-expressing CHO cells were efficiently subjected to cell death induction with the treatment culture sup. containing h2D11B-v1, h12H8B-v1, h12H8B-v2.1, h12H8C-v1, and h12H8C-v2.1.

TABLE 10
Specificity of target cell lysis (% of PI-positive target cells (untreated
background subtracted)) by different combinations of bsAbs

Cell

| | |
|---|---|
| CHO/EpCAM | CHO |

fold dilution

| | 2x | 6x | 20x | 2x | 6x | 20x |
|---|---|---|---|---|---|---|
| h2D11B-v1 | 31.4 | 30.9 | 17.3 | 0.7 | 0 | 0.1 |
| h2D11B- | 2.2 | 1.2 | 0 | 4.3 | 2.2 | 0.5 |
| v2.1 | | | | | | |
| h12H8B-v1 | 43.6 | 41.9 | 37.2 | 0.4 | 0 | 0 |
| h12H8B- | 33.2 | 27.4 | 13.5 | 1.1 | 0.1 | 0.4 |
| v2.1 | | | | | | |
| h12H8C-v1 | 46.3 | 49.4 | 43.2 | 0.2 | 0 | 0 |
| h12H8C- | 58.7 | 53.6 | 46.4 | 0.4 | 0.3 | 0.3 |
| v2.1 | | | | | | |

Parental CHO or stably transfected CHO/EpCAM were used as target cells in a cytotoxicity assay in the presence of human PBMC at a ratio of 1:5 and serial dilution of bsAb containing culture sup. Target cell lysis was determined by flow cytometry as the percentage of target cells becoming propidium iodide-positive after 20 h of incubation.

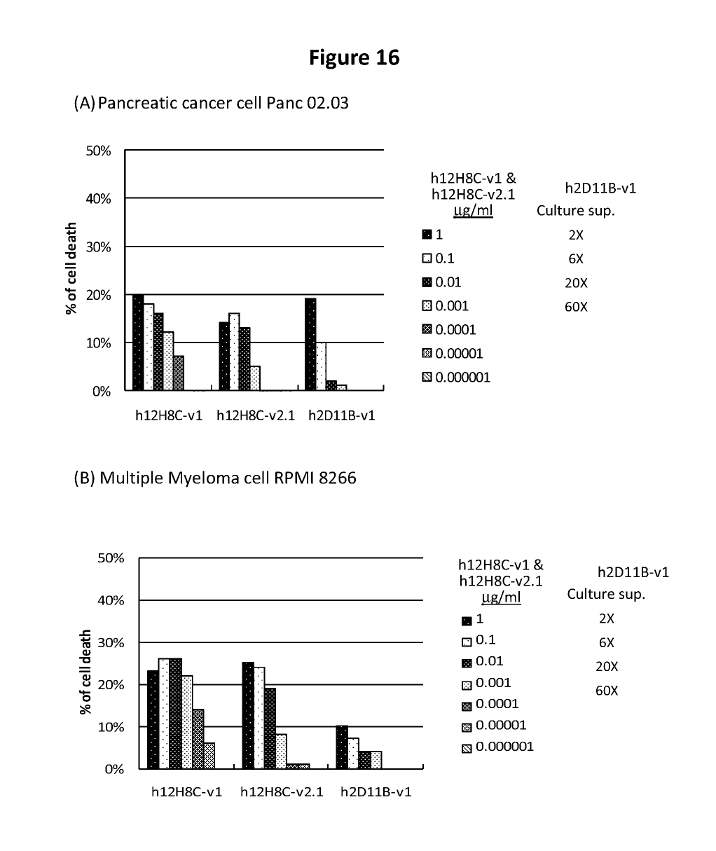

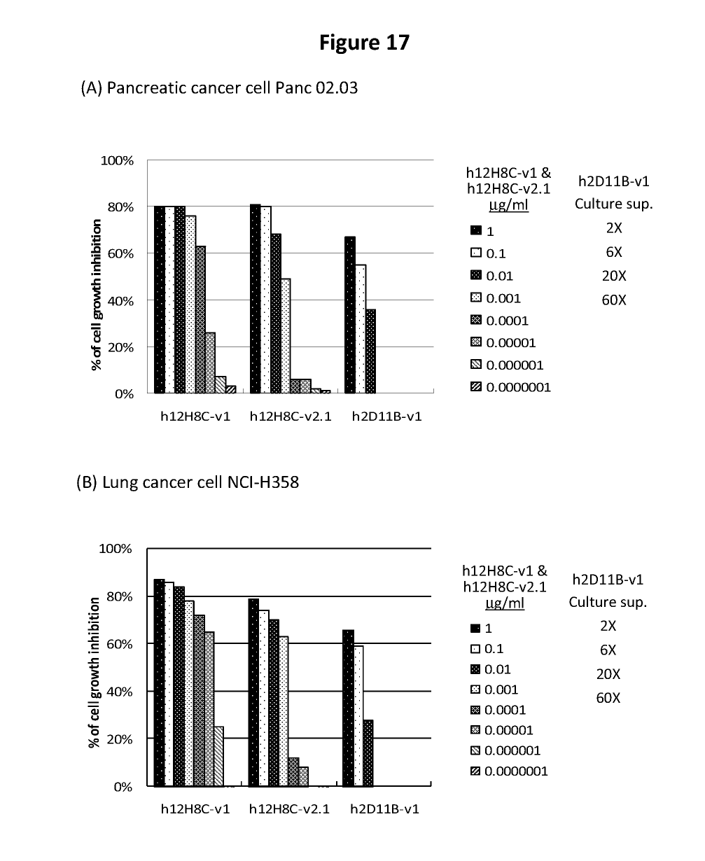

FASC-based apoptosis assay and cell proliferation assay were used to determine the extent of specific cytotoxicity of either purified bsAb or culture sup. containing bsAb against human carcinoma cells in the presence of human PBMCs. Effector cells were mixed with target cells at an effector-to-target ratio of 5:1 and incubated with serial dilutions of culture sup. or purified bsAbs for overnight. A dose-response of target cell apoptosis or growth inhibition is shown in FIGS. 16 and 17 for human pancreatic (Panc.02.03), lung cancer (NCI-H358) and Multiple Myeloma cells (RPMI 8226).

In Vivo Efficacy Study with AbGn bsAbs

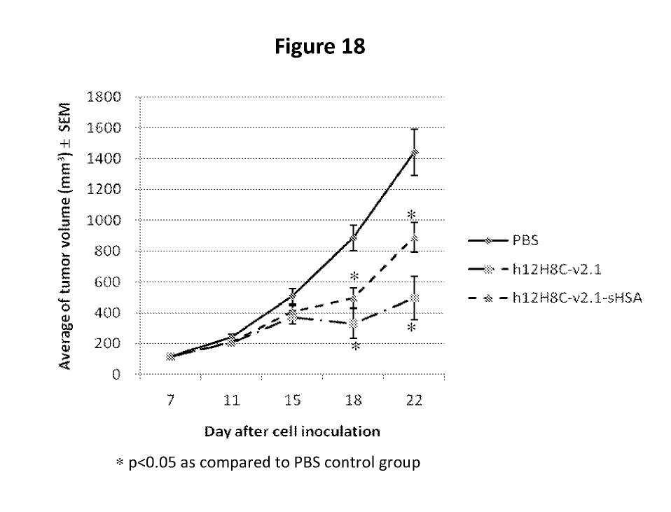

A xenografted mouse model was used to evaluate the cytotoxicity of T cell directed by bsAb h12H8C-v2.1 and h12H8C-v2.1-sHSA in vivo. Human PBMC prepared from healthy blood donor were injected directly into the tumors just prior to bsAb treatments. Mice were then divided into 3 groups which received 10 μg/mouse of h12H8C-v2.1, h12H8C-v2.1-sHSA or the vehicle (PBS) respectively, 1 hr after hPBMC intra-tumor injection. Treatment was repeated for 8 consecutive days. The outgrowth of solid subcutaneous DLD-1 tumors was determined by caliper measurements and used as an efficacy measure (FIG. 18). As shown in FIG. 18, in NOD-SCID mice bearing established DLD-1 tumors, administration of bsAbs (h12H8C-v2.1 and h12H8C-v2.1-sHSA) effectively suppressed the tumor growth compared to PBS treatment control. At day 22, the averages of tumor volumes in the two treatment groups were 494.3 mm³ and 890.0 mm³, significantly smaller compared to 1438.5 mm³ of control group.

Although the foregoing invention has been described in some detail by way of illustration and example for purposes of clarity of understanding, the descriptions and examples should not be construed as limiting the scope of the invention.

What is claimed is:

1. An isolated monoclonal antibody, which antibody specifically binds to an epitope within amino acids 24-63 of human EpCAM, wherein the naked antibody induces apoptosis of human cancer cells after binding to the epitope on the cell surface of the cancer cells in vitro, and the apoptosis-inducing activity of the naked antibody to human lung cancer cell line NCI-H358 is at least about 90% of the activity of an antibody selected from the group consisting of 12H8, 1F10, 1G10, 2D11, 6D11, and 4D2, wherein the apoptosis-inducing activity is measured by incubating the human lung cancer cell line with an antibody at concentration of about 10 ug/ml and an incubation time of about 16-20 hours.

2. The antibody of claim 1, wherein the binding of the antibody to the epitope within amino acids 24-63 of human EpCAM depends on the presence of amino acid residues selected from the group consisting of:
(1) residues Q24, E25 and N42 of human EpCAM;
(2) residues Q24, E25, E26, N37, N41, Q47, and T49 of human EpCAM;
(3) residues E25, V40 and R44 of human EpCAM;
(4) residues N41, N43, and R44 of human EpCAM; and
(5) residues Q24, E25, A35, F39, V40, N41, R44, Q45, and Q47 of human EpCAM.

3. The antibody of claim 1, which antibody induces apoptosis of human cancer cells selected from the group consisting of breast cancer cells, colorectal cancer cells, gastric cancer cells, lung cancer cells, prostate cancer cells, pancreatic cancer cells, pharynx cancer cells, and ovarian cancer cells.

4. An isolated monoclonal antibody that specifically binds to human EpCAM, comprising the three heavy chain complementary determining regions from SEQ ID NO:3, and the three light chain complementary determining regions from SEQ ID NO:5.

5. The antibody of claim 4, wherein the antibody comprises a heavy chain variable region comprising the amino acid sequence of SEQ ID NO:3, and a light chain variable region comprising the amino acid sequence of SEQ ID NO:5.

6. The antibody of claim 5, further comprising a heavy chain constant region and a light chain constant region from a human antibody.

7. The antibody of claim 4, wherein the antibody is a humanized antibody.

8. An isolated monoclonal antibody that specifically binds to human EpCAM, comprising a heavy chain variable region comprising the amino acid sequence of SEQ ID NO:3, or a light chain variable region comprising the amino acid sequence of SEQ ID NO:5.

9. An isolated monoclonal antibody that specifically binds to human EpCAM, comprising the three heavy chain complementary determining regions from SEQ ID NO:7, and the three light chain complementary determining regions from SEQ ID NO:9.

10. The antibody of claim 9, wherein the antibody comprises a heavy chain variable region comprising the amino acid sequence of SEQ ID NO:7, and a light chain variable region comprising the amino acid sequence of SEQ ID NO:9.

11. The antibody of claim 10, further comprising a heavy chain constant region and a light chain constant region from a human antibody.

12. The antibody of claim 9, wherein the antibody is a humanized antibody.

13. An isolated monoclonal antibody that specifically binds to human EpCAM, comprising a heavy chain variable region comprising the amino acid sequence of SEQ ID NO:7, or a light chain variable region comprising the amino acid sequence of SEQ ID NO:9.

14. An isolated monoclonal antibody that specifically binds to human EpCAM, comprising the three heavy chain complementary determining regions from SEQ ID NO:11, and the three light chain complementary determining regions from SEQ ID NO:13.

15. The antibody of claim 14, wherein the antibody comprises a heavy chain variable region comprising the amino acid sequence of SEQ ID NO:11, and a light chain variable region comprising the amino acid sequence of SEQ ID NO:13.

16. The antibody of claim 15, further comprising a heavy chain constant region and a light chain constant region from a human antibody.

17. The antibody of claim 14, wherein the antibody is a humanized antibody.

18. An isolated monoclonal antibody that specifically binds to human EpCAM, comprising a heavy chain variable region comprising the amino acid sequence of SEQ ID NO:11, or a light chain variable region comprising the amino acid sequence of SEQ ID NO:13.

19. An isolated monoclonal antibody that specifically binds to human EpCAM, comprising the three heavy chain complementary determining regions from SEQ ID NO:15, and the three light chain complementary determining regions from SEQ ID NO:17.

20. The antibody of claim 19, wherein the antibody comprises a heavy chain variable region comprising the amino acid sequence of SEQ ID NO:15, and a light chain variable region comprising the amino acid sequence of SEQ ID NO:17.

21. The antibody of claim 20, further comprising a heavy chain constant region and a light chain constant region from a human antibody.

22. The antibody of claim 19, wherein the antibody is a humanized antibody.

23. An isolated monoclonal antibody that specifically binds to human EpCAM, comprising a heavy chain variable region comprising the amino acid sequence of SEQ ID NO:15, or a light chain variable region comprising the amino acid sequence of SEQ ID NO:17.

24. An isolated monoclonal antibody that specifically binds to human EpCAM, comprising the three heavy chain complementary determining regions from SEQ ID NO:19, and the three light chain complementary determining regions from SEQ ID NO:21.

25. The antibody of claim 24, wherein the antibody comprises a heavy chain variable region comprising the amino acid sequence of SEQ ID NO:19, and a light chain variable region comprising the amino acid sequence of SEQ ID NO:21.

26. The antibody of claim 25, further comprising a heavy chain constant region and a light chain constant region from a human antibody.

27. The antibody of claim 24, wherein the antibody is a humanized antibody.

28. An isolated monoclonal antibody that specifically binds to human EpCAM, comprising a heavy chain variable region comprising the amino acid sequence of SEQ ID NO:19, or a light chain variable region comprising the amino acid sequence of SEQ ID NO:21.

29. An isolated monoclonal antibody that specifically binds to human EpCAM, comprising the three heavy chain complementary determining regions from SEQ ID NO:23, and the three light chain complementary determining regions from SEQ ID NO:25.

30. The antibody of claim 29, wherein the antibody comprises a heavy chain variable region comprising the amino acid sequence of SEQ ID NO:23, and a light chain variable region comprising the amino acid sequence of SEQ ID NO:25.

31. The antibody of claim 30, further comprising a heavy chain constant region and a light chain constant region from a human antibody.

32. The antibody of claim 29, wherein the antibody is a humanized antibody.

33. An isolated monoclonal antibody that specifically binds to human EpCAM, comprising a heavy chain variable region comprising the amino acid sequence of SEQ ID NO:23, or a light chain variable region comprising the amino acid sequence of SEQ ID NO:25.

34. A single-chain bispecific antibody comprising (a) a first antigen binding domain that specifically binds to an epitope within amino acids 24-63 of human EpCAM, wherein the first antigen binding domain comprises a heavy chain variable region (V_{H}EpCAM) and a light chain variable region (V_{L}EpCAM); and (b) a second antigen binding domain that specifically binds to human CD3 antigen, wherein the second antigen binding domain comprises a heavy chain variable region (V_{H}CD3) and a light chain variable region (V_{L}CD3); wherein the variable regions are arranged from N-terminus to C-terminus in the order V_{L}EpCAM-V_{H}EpCAM-V_{H}CD3-V_{L}CD3.

35. The bispecific antibody of claim 34, further comprising a peptide linker between V_{L}EpCAM and V_{H}EpCAM, between V_{H}EpCAM and V_{H}CD3, and/or between V_{H}CD3 and V_{L}CD3.

36. The bispecific antibody of claim 35, wherein the peptide linker between V_{L}EpCAM and V_{H}EpCAM comprises amino acid sequence of SEQ ID NO:49.

37. The bispecific antibody of claim 35, wherein the peptide linker between V_{H}CD3 and V_{L}CD3 comprises the amino acid sequence of SEQ ID NO:53.

38. The bispecific antibody of claim 35, wherein the peptide linker between V_{H}EpCAM and V_{H}CD3 comprises the amino acid sequence of SEQ ID NO:51.

39. The bispecific antibody of claim 34, wherein the first antigen binding domain comprises the V_{H}EpCAM and the V_{L}EpCAM selected from the group consisting of:
(a) the V_{H}EpCAM comprising the three CDRs from SEQ ID NO:3, and the V_{L}EpCAM comprising the three CDRs from SEQ ID NO:5;
(b) the V_{H}EpCAM comprising the three CDRs from SEQ ID NO:7, and the V_{L}EpCAM comprising the three CDRs from SEQ ID NO:9;
(c) the V_{H}EpCAM comprising the three CDRs from SEQ ID NO:11, and the V_{L}EpCAM comprising the three CDRs from SEQ ID NO:13;
(d) the V_{H}EpCAM comprising the three CDRs from SEQ ID NO:15, and the V_{L}EpCAM comprising the three CDRs from SEQ ID NO:17;
(e) the V_{H}EpCAM comprising the three CDRs from SEQ ID NO:19, and the V_{L}EpCAM comprising the three CDRs from SEQ ID NO:21; and
(f) the V_{H}EpCAM comprising the three CDRs from SEQ ID NO:23, and the V_{L}EpCAM comprising the three CDRs from SEQ ID NO:25.

40. The bispecific antibody of claim 39, wherein the V_{H}EpCAM and the V_{L}EpCAM are humanized.

41. The bispecific antibody of claim 40, wherein the first antigen binding domain comprises the V_{H}EpCAM and the V_{L}EpCAM selected from the group consisting of:
(a) the V_{H}EpCAM comprising the amino acid sequence of SEQ ID NO:27, and the V_{L}EpCAM comprising the amino acid sequence of SEQ ID NO:29;
(b) the V_{H}EpCAM comprising the amino acid sequence of SEQ ID NO:31, and the V_{L}EpCAM comprising the amino acid of SEQ ID NO:33; and
(c) the V_{H}EpCAM comprising the amino acid sequence of SEQ ID NO:35, and the V_{L}EpCAM comprising the amino acid sequence of SEQ ID NO:37.

42. The bispecific antibody of claim 34, wherein the second antigen binding domain specifically binds to CD3ε, CD3γ, or CD3δ chain.

43. The bispecific antibody of claim 34, wherein the second antigen binding domain comprises the V_{H}CD3 and V_{L}CD3, wherein the V_{H}CD3 comprises the amino acid sequence of SEQ ID NO:55, and wherein the V_{L}CD3 comprises the amino acid sequence of SEQ ID NO:57.

44. The bispecific antibody of claim 34, further comprising a human serum albumin sequence (HSA) at the C-terminus of the bispecific antibody.

45. The bispecific antibody of claim 44, wherein the human serum albumin sequence comprising the amino acid sequence of SEQ ID NO: 45 or SEQ ID NO:47.

46. The bispecific antibody of claim 44, further comprising a peptide linker between the V_{L}CD3 and the human serum albumin sequence.

47. The bispecific antibody of claim 46, wherein the peptide linker between the V_{L}CD3 and the human serum albumin sequence comprises the amino acid sequence of SEQ ID NO:51.

48. The bispecific antibody of claim 34, said antibody comprising the amino acid sequence selected from the group consisting of SEQ ID NO:39, and SEQ ID NO:41, and SEQ ID NO:43.

49. A pharmaceutical composition comprising the antibody of claim 1 or 34 and a pharmaceutically acceptable carrier.

50. An isolated polynucleotide comprising a nucleic acid sequence encoding the antibody of claim 1 or 34.

51. A vector comprising the polynucleotide of claim 50.

52. A host cell comprising the vector of claim 51.

53. A method of producing an antibody, comprising culturing the host cells of claim 52 that produces the antibody encoded by the nucleic acid, and recovering the antibody from the cell culture.

54. A method of screening an antibody that specifically binds human EpCAM and induces apoptosis of human cancer cells in vitro, comprising:
(a) culturing a cancer cell with an effective concentration of a naked monoclonal antibody that specifically binds to human EpCAM in vitro;
(b) measuring the apoptosis of the cancer cell induced by the naked monoclonal antibody; and
(c) selecting the antibody if the antibody has higher apoptosis-inducing activity as compared to a control antibody.

55. The method of claim 54, wherein apoptosis-inducing activity is measured by Annexin V and Propidium Iodide staining of the cancer cell.

56. The method of claim 54, wherein the cancer cell is selected from the group consisting of a breast cancer cell, a colorectal cancer cell, a gastric cancer cell, a lung cancer cell, a prostate cancer cell, a pancreatic cancer cell, a pharynx cancer cell, and an ovarian cancer cell.

57. The method of claim 54, wherein an antibody having at least 90% of the apoptosis-inducing activity as an antibody selected from the group consisting of 12H8, 1F10, 1G10, 2D11, 6D11, and 4D2 is selected.

58. A method for treating a cancer or delaying development of a cancer in an individual comprising administering to the individual an effective amount of the antibody of claim 1 or 34.

59. The method of claim 58, wherein the cancer is selected from the group consisting of breast cancer, colorectal cancer, gastric cancer, lung cancer, prostate cancer, pancreatic cancer, pharynx cancer, and ovarian cancer.

60. The method of claim 58, further comprising administering to the individual a second anti-cancer agent.

61. The method of claim 60, wherein the second anti-cancer agent is a chemotherapeutic agent.

62. The method of claim 61, wherein the second anti-cancer agent is Oxaliplatin.

63. A kit comprising the antibody of claim 1 or 34.

64. The kit of claim 63, further comprising instructions for administering an effective amount of the antibody to an individual for treating cancer in the individual.

65. The kit of claim 63, further comprising a second anti-cancer agent.

66. The kit of claim 65, further comprising instructions for administering the antibody and the second anti-cancer agent in conjunction to an individual for treating cancer in the individual.
